SEQUENCE LISTING (72 sequences)

SEQ ID NO: 1 - DNA, 1047 bp, Homo sapiens
  source 1..1047: mol_type = other DNA
atgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcagttccggaactgcatgctcaccaccatctgctgcggcaagaacccactgggtgacgatgaggcctctgctaccgtgtccaagacggagacgagccaggtggccccggcctaa

SEQ ID NO: 2 - protein, 348 aa, Homo sapiens [sequence omitted]

SEQ ID NO: 3 - DNA, 21 bp, synthetic construct
  misc_feature 1..21: note = Target sequence of mirtron 1
  source 1..21: mol_type = other DNA
atctacatgttcgtggtccac

SEQ ID NO: 4 - DNA, 21 bp, synthetic construct
  misc_feature 1..21: note = Target sequence of mirtron 2
  source 1..21: mol_type = other DNA
atcaacttcctcacgctctac

SEQ ID NO: 5 - DNA, 21 bp, synthetic construct
  misc_feature 1..21: note = Target sequence of mirtron 3
  source 1..21: mol_type = other DNA
cttccccatcaacttcctcac

SEQ ID NO: 6 - DNA, 21 bp, synthetic construct
  misc_feature 1..21: note = Target sequence of mirtron 4
  source 1..21: mol_type = other DNA
cttcctcacgctctacgtcac

SEQ ID NO: 7 - DNA, 21 bp, synthetic construct
  misc_feature 1..21: note = Target sequence of mirtron 5H
  source 1..21: mol_type = other DNA
aacgagtcttttgtcatctac

SEQ ID NO: 8 - DNA, 21 bp, synthetic construct
  misc_feature 1..21: note = Target sequence of mirtron 6
  source 1..21: mol_type = other DNA
catgttcgtggtccacttcac

SEQ ID NO: 9 - DNA, 21 bp, synthetic construct
  misc_feature 1..21: note = Target sequence of mirtron 7
  source 1..21: mol_type = other DNA
gttccggaactgcatgctcac

SEQ ID NO: 10 - DNA, 76 bp, synthetic construct
  misc_feature 1..76: note = Mirtron 1
  source 1..76: mol_type = other DNA
gtggaccacgaacatgtagatttcaagagaatctacatgttcgtggtccctcagttttttctctttcttttcgaag

SEQ ID NO: 11 - DNA, 76 bp, synthetic construct
  misc_feature 1..76: note = Mirtron 2
  source 1..76: mol_type = other DNA
gtagagcgtgaggaagttgatttcaagagaatcaacttcctcacgctttctcagttttttctctttcttttcgaag

SEQ ID NO: 12 - DNA, 76 bp, synthetic construct
  misc_feature 1..76: note = Mirtron 3
  source 1..76: mol_type = other DNA
gtgaggaagttgatggggaagttcaagagacttccccatcaacttctttctcagttttttctctttcttttcgaag

SEQ ID NO: 13 - DNA, 76 bp, synthetic construct
  misc_feature 1..76: note = Mirtron 4
  source 1..76: mol_type = other DNA
gtgacgtagagcgtgaggaagttcaagagacttcctcacgctctacgttctcagttttttctctttcttttcgaag

SEQ ID NO: 14 - DNA, 76 bp, synthetic construct
  misc_feature 1..76: note = Mirtron 5H
  source 1..76: mol_type = other DNA
gtagatgacaaaagactcgttttcaagagaaacgagtcttttgtcatttctcagttttttctctttcttttcgaag

SEQ ID NO: 15 - DNA, 76 bp, synthetic construct
  misc_feature 1..76: note = Mirtron 6
  source 1..76: mol_type = other DNA
gtgaagtggaccacgaacatgttcaagagacatgttcgtggtccactttctcagttttttctctttcttttcgaag

SEQ ID NO: 16 - DNA, 76 bp, synthetic construct
  misc_feature 1..76: note = Mirtron 7
  source 1..76: mol_type = other DNA
gtgagcatgcagttccggaacttcaagagagttccggaactgcatgtttctcagttttttctctttcttttcgaag

SEQ ID NO: 17 - DNA, 21 bp, synthetic construct
  misc_feature 1..21: note = Guide strand sequence of mirtron 1
  source 1..21: mol_type = other DNA
gtggaccacgaacatgtagat

SEQ ID NO: 18 - DNA, 21 bp, synthetic construct
  misc_feature 1..21: note = Guide strand sequence of mirtron 2
  source 1..21: mol_type = other DNA
gtagagcgtgaggaagttgat

SEQ ID NO: 19 - DNA, 21 bp, synthetic construct
  misc_feature 1..21: note = Guide strand sequence of mirtron 3
  source 1..21: mol_type = other DNA
gtgaggaagttgatggggaag

SEQ ID NO: 20 - DNA, 21 bp, synthetic construct
  misc_feature 1..21: note = Guide strand sequence of mirtron 4
  source 1..21: mol_type = other DNA
gtgacgtagagcgtgaggaag

SEQ ID NO: 21 - DNA, 21 bp, synthetic construct
  misc_feature 1..21: note = Guide strand sequence of mirtron 5
  source 1..21: mol_type = other DNA
gtagatgacaaaagactcgtt

SEQ ID NO: 22 - DNA, 21 bp, synthetic construct
  misc_feature 1..21: note = Guide strand sequence of mirtron 6
  source 1..21: mol_type = other DNA
gtgaagtggaccacgaacatg

SEQ ID NO: 23 - DNA, 21 bp, synthetic construct
  misc_feature 1..21: note = Guide strand sequence of mirtron 7
  source 1..21: mol_type = other DNA
gtgagcatgcagttccggaac

SEQ ID NO: 24 - DNA, 21 bp, synthetic construct
  misc_feature 1..21: note = Modified target sequence of mirtron 3
  source 1..21: mol_type = other DNA
atttccaattaattttctgac

SEQ ID NO: 25 - DNA, 21 bp, synthetic construct
  misc_feature 1..21: note = Modified target sequence of mirtron 5H
  source 1..21: mol_type = other DNA
aatgaatccttcgtgatttat

SEQ ID NO: 26 - DNA, 340 bp, synthetic construct
  misc_feature 1..340: note = 5'UTR insert (eGFP - M3 - eGFP - M5 - eGFP)
  source 1..340: mol_type = other DNA
tgacgggaactacaagacccgcgctgaagtcaagttcgaaggtgaggaagttgatggggaagttcaagagacttccccatcaacttctttctcagttttttctctttcttttcgaaggtgacaccctggtgaatagaatcgagctgaagggcattgactttaaggaggattgacgggaactacaagacccgcgctgaagtcaagttcgaaggtagatgacaaaagactcgttttcaagagaaacgagtcttttgtcatttctcagttttttctctttcttttcgaaggtgacaccctggtgaatagaatcgagctgaagggcattgactttaaggaggat

SEQ ID NO: 27 - DNA, 779 bp, synthetic construct
  misc_feature 1..779: note = 5'UTR with mirtrons and additional ESE-rich sequences
  source 1..779: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatcctagcttgattgacgggaactacaagacccgcgctgaagtcaagttcgaaggtgaggaagttgatggggaagttcaagagacttccccatcaacttctttctcagttttttctctttcttttcgaaggtgacaccctggtgaatagaatcgagctgaagggcattgactttaaggaggattgacgggaactacaagacccgcgctgaagtcaagttcgaaggtagatgacaaaagactcgttttcaagagaaacgagtcttttgtcatttctcagttttttctctttcttttcgaaggtgacaccctggtgaatagaatcgagctgaagggcattgactttaaggaggatatcgaattccgccacc

SEQ ID NO: 28 - DNA, 3947 bp, synthetic construct
  misc_feature 1..3947: note = AAV sequence
  source 1..3947: mol_type = other DNA
cacatacgatttaggtgacactatagaatacacggaattaattctagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtacccagatcttccccacctagccacctggcaaactgctccttctctcaaaggcccaaacatggcctcccagactgcaacccccaggcagtcaggccctgtctccacaacctcacagccaccctggacggaatctgcttcttcccacatttgagtcctcctcagcccctgagctcctctgggcagggctgtttctttccatctttgtattcccaggggcctgcaaataaatgtttaatgaacgaacaagagagtgaattccaattccatgcaacaaggattgggctcctgggccctaggctatgtgtctggcaccagaaacggaagctgcaggttgcagcccctgccctcatggagctcctcctgtcagaggagtgtggggactggatgactccagaggtaacttgtgggggaacgaacaggtaaggggctgtgtgacgagatgagagactgggagaataaaccagaaagtctctagctgtccagaggacatagcacagaggcccatggtccctatttcaaacccaggccaccagactgagctgggaccttgggacagacaagtcatgcagaagttaggggaccttctcctcccttttcctggatcctgagtacctctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtcatccggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatcctagcttgattgacgggaactacaagacccgcgctgaagtcaagttcgaaggtgaggaagttgatggggaagttcaagagacttccccatcaacttctttctcagttttttctctttcttttcgaaggtgacaccctggtgaatagaatcgagctgaagggcattgactttaaggaggattgacgggaactacaagacccgcgctgaagtcaagttcgaaggtagatgacaaaagactcgttttcaagagaaacgagtcttttgtcatttctcagttttttctctttcttttcgaaggtgacaccctggtgaatagaatcgagctgaagggcattgactttaaggaggatatcgaattccgccaccatgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggatttccaattaattttctgacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtacatccccgagggcctgcagtgctcgtgtggaatcgactactacacgctcaagccggaggtcaacaatgaatccttcgtgatttatatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcagttccggaactgcatgctcaccaccatctgctgcggcaagaacccactgggtgacgatgaggcctctgctaccgtgtccaagacggagacgagccaggtggccccggcctaaaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggcggcctcagtgagcgagcgagcgcgcagagctttttgcaaaagccta

SEQ ID NO: 29 - DNA, 589 bp, synthetic construct
  misc_feature 1..589: note = WPRE
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 30 - skipped

SEQ ID NO: 31 - DNA, 7012 bp, synthetic construct
  misc_feature 1..7012: note = Gene therapy vector
  source 1..7012: mol_type = other DNA
cacatacgatttaggtgacactatagaatacacggaattaattctagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtacccagatcttccccacctagccacctggcaaactgctccttctctcaaaggcccaaacatggcctcccagactgcaacccccaggcagtcaggccctgtctccacaacctcacagccaccctggacggaatctgcttcttcccacatttgagtcctcctcagcccctgagctcctctgggcagggctgtttctttccatctttgtattcccaggggcctgcaaataaatgtttaatgaacgaacaagagagtgaattccaattccatgcaacaaggattgggctcctgggccctaggctatgtgtctggcaccagaaacggaagctgcaggttgcagcccctgccctcatggagctcctcctgtcagaggagtgtggggactggatgactccagaggtaacttgtgggggaacgaacaggtaaggggctgtgtgacgagatgagagactgggagaataaaccagaaagtctctagctgtccagaggacatagcacagaggcccatggtccctatttcaaacccaggccaccagactgagctgggaccttgggacagacaagtcatgcagaagttaggggaccttctcctcccttttcctggatcctgagtacctctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtcatccggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatcctagcttgattgacgggaactacaagacccgcgctgaagtcaagttcgaaggtgaggaagttgatggggaagttcaagagacttccccatcaacttctttctcagttttttctctttcttttcgaaggtgacaccctggtgaatagaatcgagctgaagggcattgactttaaggaggattgacgggaactacaagacccgcgctgaagtcaagttcgaaggtagatgacaaaagactcgttttcaagagaaacgagtcttttgtcatttctcagttttttctctttcttttcgaaggtgacaccctggtgaatagaatcgagctgaagggcattgactttaaggaggatatcgaattccgccaccatgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggatttccaattaattttctgacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtacatccccgagggcctgcagtgctcgtgtggaatcgactactacacgctcaagccggaggtcaacaatgaatccttcgtgatttatatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcagttccggaactgcatgctcaccaccatctgctgcggcaagaacccactgggtgacgatgaggcctctgctaccgtgtccaagacggagacgagccaggtggccccggcctaaaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggcggcctcagtgagcgagcgagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcata

SEQ ID NO: 32 - DNA, 177 bp, synthetic construct
  misc_feature 1..177: note = Forward ITR
  source 1..177: mol_type = other DNA
cacatacgatttaggtgacactatagaatacacggaattaattctagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 33 - DNA, 137 bp, synthetic construct
  misc_feature 1..137: note = Reverse ITR
  source 1..137: mol_type = other DNA
ggaacccctagtgatggagttggccactccctctctgcgcgctcgctcgctcactgaggccgggcgaccaaaggtcgcccgacgcccgggcggcctcagtgagcgagcgagcgcgcagagctttttgcaaaagccta

SEQ ID NO: 34 - DNA, 806 bp, synthetic construct
  misc_feature 1..806: note = Forward ITR human rhodopsin promoter
  source 1..806: mol_type = other DNA
cagatcttccccacctagccacctggcaaactgctccttctctcaaaggcccaaacatggcctcccagactgcaacccccaggcagtcaggccctgtctccacaacctcacagccaccctggacggaatctgcttcttcccacatttgagtcctcctcagcccctgagctcctctgggcagggctgtttctttccatctttgtattcccaggggcctgcaaataaatgtttaatgaacgaacaagagagtgaattccaattccatgcaacaaggattgggctcctgggccctaggctatgtgtctggcaccagaaacggaagctgcaggttgcagcccctgccctcatggagctcctcctgtcagaggagtgtggggactggatgactccagaggtaacttgtgggggaacgaacaggtaaggggctgtgtgacgagatgagagactgggagaataaaccagaaagtctctagctgtccagaggacatagcacagaggcccatggtccctatttcaaacccaggccaccagactgagctgggaccttgggacagacaagtcatgcagaagttaggggaccttctcctcccttttcctggatcctgagtacctctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtcatcc

SEQ ID NO: 35 - DNA, 717 bp, synthetic construct
  misc_feature 1..717: note = eGFP CDS
  source 1..717: mol_type = other DNA
atgagcaagggcgaggaactgttcactggcgtggtcccaattctcgtggaactggatggcgatgtgaatgggcacaaattttctgtcagcggagagggtgaaggtgatgccacatacggaaagctcaccctgaaattcatctgcaccactggaaagctccctgtgccatggccaacactggtcactaccctgacctatggcgtgcagtgcttttccagatacccagaccatatgaagcagcatgactttttcaagagcgccatgcccgagggctatgtgcaggagagaaccatctttttcaaagatgacgggaactacaagacccgcgctgaagtcaagttcgaaggtgacaccctggtgaatagaatcgagctgaagggcattgactttaaggaggatggaaacattctcggccacaagctggaatacaactataactcccacaatgtgtacatcatggccgacaagcaaaagaatggcatcaaggtcaacttcaagatcagacacaacattgaggatggatccgtgcagctggccgaccattatcaacagaacactccaatcggcgacggccctgtgctcctcccagacaaccattacctgtccacccagtctgccctgtctaaagatcccaacgaaaagagagaccacatggtcctgctggagtttgtgaccgctgctgggatcacacatggcatggacgagctgtacaagtga

SEQ ID NO: 36 - protein, 238 aa, synthetic construct
  REGION 1..238: note = eGFP amino acid
MSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 37 - DNA, 41 bp, synthetic construct
  misc_feature 1..41: note = 5' ESE rich sequence
  source 1..41: mol_type = other DNA
tgacgggaactacaagacccgcgctgaagtcaagttcgaag

SEQ ID NO: 38 - DNA, 53 bp, synthetic construct
  misc_feature 1..53: note = 3' ESE rich sequence
  source 1..53: mol_type = other DNA
gtgacaccctggtgaatagaatcgagctgaagggcattgactttaaggaggat

SEQ ID NO: 39 - DNA, 439 bp, synthetic construct
  misc_feature 1..439: note = 5'UTR without mirtrons and additional ESE-rich sequences
  source 1..439: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatcctagcttgatatcgaattccgccacc

SEQ ID NO: 40 - DNA, 94 bp, synthetic construct
  misc_feature 1..94: note = ESE-rich sequence between tandem mirtrons
  source 1..94: mol_type = other DNA
gtgacaccctggtgaatagaatcgagctgaagggcattgactttaaggaggattgacgggaactacaagacccgcgctgaagtcaagttcgaag

SEQ ID NO: 41 - DNA, 76 bp, synthetic construct
  misc_feature 1..76: note = Mirtron 1 forward oligonucleotide
  source 1..76: mol_type = other DNA
cgaaggtggaccacgaacatgtagatttcaagagaatctacatgttcgtggtccctcagttttttctctttctttt

SEQ ID NO: 42 - DNA, 76 bp, synthetic construct
  misc_feature 1..76: note = Mirtron 2 forward oligonucleotide
  source 1..76: mol_type = other DNA
cgaaggtagagcgtgaggaagttgatttcaagagaatcaacttcctcacgctttctcagttttttctctttctttt

SEQ ID NO: 43 - DNA, 76 bp, synthetic construct
  misc_feature 1..76: note = Mirtron 3 forward oligonucleotide
  source 1..76: mol_type = other DNA
cgaaggtgaggaagttgatggggaagttcaagagacttccccatcaacttctttctcagttttttctctttctttt

SEQ ID NO: 44 - DNA, 76 bp, synthetic construct
  misc_feature 1..76: note = Mirtron 4 forward oligonucleotide
  source 1..76: mol_type = other DNA
cgaaggtgacgtagagcgtgaggaagttcaagagacttcctcacgctctacgttctcagttttttctctttctttt

SEQ ID NO: 45 - DNA, 76 bp, synthetic construct
  misc_feature 1..76: note = Mirtron 5H forward oligonucleotide
  source 1..76: mol_type = other DNA
cgaaggtagatgacaaaagactcgttttcaagagaaacgagtcttttgtcatttctcagttttttctctttctttt

SEQ ID NO: 46 - DNA, 76 bp, synthetic construct
  misc_feature 1..76: note = Mirtron 6 forward oligonucleotide
  source 1..76: mol_type = other DNA
cgaaggtgaagtggaccacgaacatgttcaagagacatgttcgtggtccactttctcagttttttctctttctttt

SEQ ID NO: 47 - DNA, 76 bp, synthetic construct
  misc_feature 1..76: note = Mirtron 7 forward oligonucleotide
  source 1..76: mol_type = other DNA
cgaaggtgagcatgcagttccggaacttcaagagagttccggaactgcatgtttctcagttttttctctttctttt

SEQ ID NO: 48 - DNA, 76 bp, synthetic construct
  misc_feature 1..76: note = Mirtron 1 reverse oligonucleotide
  source 1..76: mol_type = other DNA
cgaaaagaaagagaaaaaactgagggaccacgaacatgtagattctcttgaaatctacatgttcgtggtccacctt

SEQ ID NO: 49 - DNA, 76 bp, synthetic construct
  misc_feature 1..76: note = Mirtron 2 reverse oligonucleotide
  source 1..76: mol_type = other DNA
cgaaaagaaagagaaaaaactgagaaagcgtgaggaagttgattctcttgaaatcaacttcctcacgctctacctt

SEQ ID NO: 50 - DNA, 76 bp, synthetic construct
  misc_feature 1..76: note = Mirtron 3 reverse oligonucleotide
  source 1..76: mol_type = other DNA
cgaaaagaaagagaaaaaactgagaaagaagttgatggggaagtctcttgaacttccccatcaacttcctcacctt

SEQ ID NO: 51 - DNA, 76 bp, synthetic construct
  misc_feature 1..76: note = Mirtron 4 reverse oligonucleotide
  source 1..76: mol_type = other DNA
cgaaaagaaagagaaaaaactgagaacgtagagcgtgaggaagtctcttgaacttcctcacgctctacgtcacctt

SEQ ID NO: 52 - DNA, 76 bp, synthetic construct
  misc_feature 1..76: note = Mirtron 5H reverse oligonucleotide
  source 1..76: mol_type = other DNA
cgaaaagaaagagaaaaaactgagaaatgacaaaagactcgtttctcttgaaaacgagtcttttgtcatctacctt

SEQ ID NO: 53 - DNA, 76 bp, synthetic construct
  misc_feature 1..76: note = Mirtron 6 reverse oligonucleotide
  source 1..76: mol_type = other DNA
cgaaaagaaagagaaaaaactgagaaagtggaccacgaacatgtctcttgaacatgttcgtggtccacttcacctt

SEQ ID NO: 54 - DNA, 76 bp, synthetic construct
  misc_feature 1..76: note = Mirtron 7 reverse oligonucleotide
  source 1..76: mol_type = other DNA
cgaaaagaaagagaaaaaactgagaaacatgcagttccggaactctcttgaagttccggaactgcatgctcacctt

SEQ ID NO: 55 - DNA, 76 bp, synthetic construct
  misc_feature 1..76: note = Mirtron 5M
  source 1..76: mol_type = other DNA
gtagatgacaaaggattcgttttcaagagaaacgaatcctttgtcatttctcagttttttctctttcttttcgaag

SEQ ID NO: 56 - DNA, 76 bp, synthetic construct
  misc_feature 1..76: note = Mirtron 5M forward oligonucleotide
  source 1..76: mol_type = other DNA
cgaaggtagatgacaaaggattcgttttcaagagaaacgaatcctttgtcatttctcagttttttctctttctttt

SEQ ID NO: 57 - DNA, 76 bp, synthetic construct
  misc_feature 1..76: note = Mirtron 5M reverse oligonucleotide
  source 1..76: mol_type = other DNA
cgaaaagaaagagaaaaaactgagaaatgacaaaggattcgtttctcttgaaaacgaatcctttgtcatctacctt

SEQ ID NO: 58 - DNA, 76 bp, synthetic construct
  misc_feature 1..76: note = Mirtron M2U-M2
  source 1..76: mol_type = other DNA
atagagcgtgaggaagttgatttcaagagaatcaacttcctcacgctttctcagttttttctctttcttttcgaag

SEQ ID NO: 59 - DNA, 76 bp, synthetic construct
  misc_feature 1..76: note = Mirtron M2U-M2 forward oligonucleotide
  source 1..76: mol_type = other DNA
cgaagatagagcgtgaggaagttgatttcaagagaatcaacttcctcacgctttctcagttttttctctttctttt

SEQ ID NO: 60 - DNA, 76 bp, synthetic construct
  misc_feature 1..76: note = Mirtron M2U-M2 reverse oligonucleotide
  source 1..76: mol_type = other DNA
cgaaaagaaagagaaaaaactgagaaagcgtgaggaagttgattctcttgaaatcaacttcctcacgctctatctt

SEQ ID NO: 61 - DNA, 76 bp, synthetic construct
  misc_feature 1..76: note = Mirtron Scr-M2
  source 1..76: mol_type = other DNA
gtagagatgtggttgaggattttcaagagaaatcctcaaccacatctttctcagttttttctctttcttttcgaag

SEQ ID NO: 62 - DNA, 76 bp, synthetic construct
  misc_feature 1..76: note = Mirtron Scr-M2 forward oligonucleotide
  source 1..76: mol_type = other DNA
cgaaggtagagatgtggttgaggattttcaagagaaatcctcaaccacatctttctcagttttttctctttctttt

SEQ ID NO: 63 - DNA, 76 bp, synthetic construct
  misc_feature 1..76: note = Mirtron Scr-M2 reverse oligonucleotide
  source 1..76: mol_type = other DNA
cgaaaagaaagagaaaaaactgagaaagatgtggttgaggatttctcttgaaaatcctcaaccacatctctacctt

SEQ ID NO: 64 - DNA, 6417 bp, synthetic construct
  misc_feature 1..6417: note = vector AAV2/8Y733F.RHOp.Ex/Int.M3/M5.RHOM3/5R
  source 1..6417: mol_type = other DNA
cacatacgatttaggtgacactatagaatacacggaattaattctagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtacccagatcttccccacctagccacctggcaaactgctccttctctcaaaggcccaaacatggcctcccagactgcaacccccaggcagtcaggccctgtctccacaacctcacagccaccctggacggaatctgcttcttcccacatttgagtcctcctcagcccctgagctcctctgggcagggctgtttctttccatctttgtattcccaggggcctgcaaataaatgtttaatgaacgaacaagagagtgaattccaattccatgcaacaaggattgggctcctgggccctaggctatgtgtctggcaccagaaacggaagctgcaggttgcagcccctgccctcatggagctcctcctgtcagaggagtgtggggactggatgactccagaggtaacttgtgggggaacgaacaggtaaggggctgtgtgacgagatgagagactgggagaataaaccagaaagtctctagctgtccagaggacatagcacagaggcccatggtccctatttcaaacccaggccaccagactgagctgggaccttgggacagacaagtcatgcagaagttaggggaccttctcctcccttttcctggatcctgagtacctctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtcatccggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatcctagcttgattgacgggaactacaagacccgcgctgaagtcaagttcgaaggtgaggaagttgatggggaagttcaagagacttccccatcaacttctttctcagttttttctctttcttttcgaaggtgacaccctggtgaatagaatcgagctgaagggcattgactttaaggaggattgacgggaactacaagacccgcgctgaagtcaagttcgaaggtagatgacaaaagactcgttttcaagagaaacgagtcttttgtcatttctcagttttttctctttcttttcgaaggtgacaccctggtgaatagaatcgagctgaagggcattgactttaaggaggatatcgaattccgccaccatgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggatttccaattaattttctgacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtacatccccgagggcctgcagtgctcgtgtggaatcgactactacacgctcaagccggaggtcaacaatgaatccttcgtgatttatatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcagttccggaactgcatgctcaccaccatctgctgcggcaagaacccactgggtgacgatgaggcctctgctaccgtgtccaagacggagacgagccaggtggccccggcctaaaagcttatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggcggcctcagtgagcgagcgagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcata

SEQ ID NO: 65 - DNA, 6417 bp, synthetic construct
  misc_feature 1..6417: note = AAV2/8Y733F.RHOp.Ex/Int.M3/M5M.RHOM3/5R
  source 1..6417: mol_type = other DNA
cacatacgatttaggtgacactatagaatacacggaattaattctagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtacccagatcttccccacctagccacctggcaaactgctccttctctcaaaggcccaaacatggcctcccagactgcaacccccaggcagtcaggccctgtctccacaacctcacagccaccctggacggaatctgcttcttcccacatttgagtcctcctcagcccctgagctcctctgggcagggctgtttctttccatctttgtattcccaggggcctgcaaataaatgtttaatgaacgaacaagagagtgaattccaattccatgcaacaaggattgggctcctgggccctaggctatgtgtctggcaccagaaacggaagctgcaggttgcagcccctgccctcatggagctcctcctgtcagaggagtgtggggactggatgactccagaggtaacttgtgggggaacgaacaggtaaggggctgtgtgacgagatgagagactgggagaataaaccagaaagtctctagctgtccagaggacatagcacagaggcccatggtccctatttcaaacccaggccaccagactgagctgggaccttgggacagacaagtcatgcagaagttaggggaccttctcctcccttttcctggatcctgagtacctctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtcatccggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatcctagcttgattgacgggaactacaagacccgcgctgaagtcaagttcgaaggtgaggaagttgatggggaagttcaagagacttccccatcaacttctttctcagttttttctctttcttttcgaaggtgacaccctggtgaatagaatcgagctgaagggcattgactttaaggaggattgacgggaactacaagacccgcgctgaagtcaagttcgaaggtagatgacaaaggattcgttttcaagagaaacgaatcctttgtcatttctcagttttttctctttcttttcgaaggtgacaccctggtgaatagaatcgagctgaagggcattgactttaaggaggatatcgaattccgccaccatgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggatttccaattaattttctgacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtacatccccgagggcctgcagtgctcgtgtggaatcgactactacacgctcaagccggaggtcaacaatgaatccttcgtgatttatatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcagttccggaactgcatgctcaccaccatctgctgcggcaagaacccactgggtgacgatgaggcctctgctaccgtgtccaagacggagacgagccaggtggccccggcctaaaagcttatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggcggcctcagtgagcgagcgagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcata

SEQ ID NO: 66 - DNA, 31 bp, synthetic construct
  misc_feature 1..31: note = 5' ESE rich sequence
  source 1..31: mol_type = other DNA
tacaagacccgcgctgaagtcaagttcgaag

SEQ ID NO: 67 - DNA, 160 bp, synthetic construct
  misc_feature 1..160: note = 5'UTR insert (eGFP - M3 - eGFP - M5 - eGFP)
  source 1..160: mol_type = other DNA
tacaagacccgcgctgaagtcaagttcgaaggtgaggaagttgatggggaagttcaagagacttccccatcaacttctttctcagttttttctctttcttttcgaaggtgacaccctggtgaatagaatcgagctgaagggcattgactttaaggaggat

SEQ ID NO: 68 - DNA, 27 bp, synthetic construct
  misc_feature 1..27: note = 5' ESE rich sequence
  source 1..27: mol_type = other DNA
agacccgcgctgaagtcaagttcgaag

SEQ ID NO: 69 - DNA, 58 bp, synthetic construct
  misc_feature 1..58: note = 3' ESE rich sequence
  source 1..58: mol_type = other DNA
gtgacaccctggtgaatagaatcgagctgaagggcattgactttaaggaggatatcga

SEQ ID NO: 70 - DNA, 161 bp, synthetic construct
  misc_feature 1..161: note = 5'UTR insert (eGFP - M3 - eGFP - M5 - eGFP)
  source 1..161: mol_type = other DNA
agacccgcgctgaagtcaagttcgaaggtagatgacaaaagactcgttttcaagagaaacgagtcttttgtcatttctcagttttttctctttcttttcgaaggtgacaccctggtgaatagaatcgagctgaagggcattgactttaaggaggatatcga

SEQ ID NO: 71 - protein, 5 aa, synthetic construct
  REGION 1..5: note = APVAT link peptide
APVAT

SEQ ID NO: 72 - DNA, 6596 bp, Synthetic construct
  source 1..6596: mol_type = other DNA
cacatacgatttaggtgacactatagaatacacggaattaattctagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtacccagatcttccccacctagccacctggcaaactgctccttctctcaaaggcccaaacatggcctcccagactgcaacccccaggcagtcaggccctgtctccacaacctcacagccaccctggacggaatctgcttcttcccacatttgagtcctcctcagcccctgagctcctctgggcagggctgtttctttccatctttgtattcccaggggcctgcaaataaatgtttaatgaacgaacaagagagtgaattccaattccatgcaacaaggattgggctcctgggccctaggctatgtgtctggcaccagaaacggaagctgcaggttgcagcccctgccctcatggagctcctcctgtcagaggagtgtggggactggatgactccagaggtaacttgtgggggaacgaacaggtaaggggctgtgtgacgagatgagagactgggagaataaaccagaaagtctctagctgtccagaggacatagcacagaggcccatggtccctatttcaaacccaggccaccagactgagctgggaccttgggacagacaagtcatgcagaagttaggggaccttctcctcccttttcctggatcctgagtacctctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtcatccggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatcctagcttgattgacgggaactacaagacccgcgctgaagtcaagttcgaaggtgaggaagttgatggggaagttcaagagacttccccatcaacttctttctcagttttttctctttcttttcgaaggtgacaccctggtgaatagaatcgagctgaagggcattgactttaaggaggattgacgggaactacaagacccgcgctgaagtcaagttcgaaggtagatgacaaaagactcgttttcaagagaaacgagtcttttgtcatttctcagttttttctctttcttttcgaaggtgacaccctggtgaatagaatcgagctgaagggcattgactttaaggaggatatctgacgggaactacaagacccgcgctgaagtcaagttcgaaggtgaggaagttgatggggaagttcaagagacttccccatcaacttctttctcagttttttctctttcttttcgaaggtgacaccctggtgaatagaatcgagctgaagggcattgactttaaggaggatatcgaattccgccaccatgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggatttccaattaattttctgacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtacatccccgagggcctgcagtgctcgtgtggaatcgactactacacgctcaagccggaggtcaacaatgaatccttcgtgatttatatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcagttccggaactgcatgctcaccaccatctgctgcggcaagaacccactgggtgacgatgaggcctctgctaccgtgtccaagacggagacgagccaggtggccccggcctaaaagcttatcgatatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggcggcctcagtgagcgagcgagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcata

(19) United States
(12) Patent Application Publication
(10) Pub. No.: US 2026/0273103 A1
(43) Pub. Date: Sep. 17, 2026

(54) GENE THERAPY

(71) Applicant: Oxford University Innovation Limited, Oxford (Oxfordshire) (GB)

(72) Inventors: Harry ORLANS, Oxford (Oxfordshire) (GB); Robert E. MACLAREN, Oxford (Oxfordshire) (GB)

(21) Appl. No.: 19/690,865

(22) Filed: May 28, 2026

Related U.S. Application Data
(63) Continuation-in-part of application No. 17/288,573, filed on Oct. 25, 2019, now abandoned.
(63) Continuation-in-part of application No. 17/288,574, filed on Oct. 25, 2019, now Pat. No. 12,649,007.

(30) Foreign Application Priority Data
Oct. 26, 2018 (GB) 1817469.8
Oct. 26, 2018 (GB) 1817470.6

(51) Int. Cl. A61K 48/00; A61K 38/00; A61P 27/02; C07K 14/72; C12N 15/86

(52) U.S. Cl. CPC A61K 48/005; A61P 27/02; C07K 14/723; C12N 15/86; A61K 38/00; C12N 2750/14143

(57) ABSTRACT

A method of gene therapy in a subject in need thereof, the method comprising administering to the subject a vector that comprises a transgene for expression in the subject and a mirtron for knocking down expression of a gene in the subject, wherein the mirtron is in the 5′UTR of the transgene. Also a vector comprising a transgene for expression from the vector and a mirtron for knocking down expression of a gene, wherein the mirtron is in the 5′UTR of the transgene and wherein the 5′UTR further comprises an exonic splice enhancer motif and/or wherein the mirtron is flanked in the 5′UTR by a fragment of the coding region of a reporter gene, and methods for preparing a vector.

### CROSS-REFERENCE TO RELATED APPLICATIONS

This application is a continuation-in-part application of U.S. Ser. No. 17/288,573, which is the National Stage of International Application No. PCT/GB2019/053037, filed Oct. 25, 2019, which claims priority to GB 1817470.6, filed Oct. 26, 2018, all of which are entirely incorporated herein by reference.

This application is also a continuation-in-part application of U.S. Ser. No. 17/288,574, which is the National Stage of International Application No. PCT/GB2019/053036, filed Oct. 25, 2019, which claims priority to GB 1817469.8, filed Oct. 26, 2018, all of which are entirely incorporated herein by reference.

### SEQUENCE LISTING

This application contains a sequence listing filed in electronic form as a st.26.xml file entitled “Sequence_Listing_N414420US_A.xml,” having a size of 128,318 bytes, created May 28, 2026. The content of the sequence listing is incorporated herein in its entirety.

### FIELD

The disclosure relates to methods of gene therapy using mirtrons and to vectors for use therein.

### BACKGROUND

A mirtron is a microRNAs derived from an intron that is released following splicing and processed within the cell. It has been proposed to use artificial mirtrons as part of a gene therapy vector to treat dominant disease. The mirtron could be accompanied in the vector by a codon-modified version of the target gene, which is engineered to be resistant to co-delivered mirtrons, as part of a ‘block and replace’ treatment paradigm. However, co-delivery of mirtrons with a transgene in a way that preserves both efficient splicing of the mirtron and adequate transgene expression is challenging (Curtis et al. Nucleic Acids Research 45(13): 7870-7885 (2017)) and has not been achieved to date.

### SUMMARY

The inventors have succeeded in achieving co-delivery of an artificial mirtron with a ‘replacement’ transcript in which expression of the transcript is not substantially adversely affected by the presence of the mirtron. The inventors achieved efficient splicing and gene knockdown using an artificial mirtron delivered in the 5′UTR of a transgene. To our knowledge this is the first demonstration of co-delivery of an efficiently spliced artificial mirtron with high levels of transgene expression. The inventors have demonstrated the first introduction of exogenous sequences containing exonic splice enhancers adjacent to a mirtron to promote or maintain efficient splicing out of a mirtron. Moreover efficient splicing can be maintained when moving a mirtron from one location, such as the coding region (CDS) of a reporter gene, to another location, such as the 5′UTR of a transgene for co-expression, by co-transferring an ESE-rich flanking sequence. The inventors have demonstrated successful use of mirtrons in vivo and to treat a disease model, the use of adeno-associated virus (AAV) to successfully deliver mirtrons, and increased gene knock-down by co-delivery of multiple mirtrons from a single transcript.

Accordingly, in a first aspect the invention provides a method of gene therapy in a subject in need thereof, the method comprising administering to the subject a vector that comprises a transgene for expression in the subject and a mirtron for knocking down expression of a gene in the subject, wherein the mirtron is in the 5′UTR of the transgene.

The invention also provides:
A vector for use in a method of treatment or gene therapy in a subject in need thereof, wherein the vector comprises a transgene for expression in the subject and a mirtron for knocking down expression of a gene in the subject, wherein the mirtron is in the 5′UTR of the transgene; and
A vector for use in the manufacture of a medicament, wherein the vector comprises a transgene and a mirtron for knocking down expression of a gene, and wherein the mirtron is in the 5′UTR of the transgene.

The 5′UTR may further comprise one or more exonic splice enhancer motifs (ESEs).

In some cases the mirtron is flanked in the 5′UTR by a fragment of the coding region of a reporter gene.

In a further aspect the invention provides a vector comprising a transgene for expression from the vector and a mirtron for knocking down expression of a gene, wherein the mirtron is in the 5′UTR of the transgene and wherein the 5′UTR further comprises exonic splice enhancers and/or wherein the mirtron is flanked in the 5′UTR by a fragment of the coding region of a reporter gene.

In a further aspect the invention provides a method for introducing a mirtron into a plasmid or vector for expression of a transgene, the method comprising inserting into the 5′UTR of the transgene a nucleotide sequence that comprises the mirtron and an upstream and/or downstream flanking sequence that is a fragment of a reporter gene and/or that comprises an exonic splice enhancer motif (ESE).

In a further aspect the invention provides a method of preparing a plasmid or vector, the method comprising:
(a) expressing a mirtron from a first plasmid or vector, wherein the mirtron is embedded in the coding region of a reporter gene in the plasmid or vector;
(b) assessing the efficiency and/or accuracy of splicing of the mirtron from the reporter gene;
(c) excising or amplifying a section from the first plasmid or vector, wherein the section comprises the mirtron and a flanking fragment of the coding region of the reporter gene; and
(d) inserting the excised or amplified section of the first plasmid or vector into a second plasmid or vector, wherein the second plasmid or vector comprises a transgene, and wherein the excised section of the first plasmid or vector is inserted in the 5′UTR of the transgene in the second plasmid or vector.

In some cases the transgene is a variant of the gene that is targeted for knock down by the mirtron, and the variant gene is resistant to knock-down by the mirtron.

In some cases the the 5′UTR comprises two or more mirtrons for knocking down expression of one or more target genes.

In a further aspect the invention provides a pharmaceutical composition comprising a gene therapy vector as described above.

In an example, described herein is a triple mirtron polynucleotide, gene therapy vector comprising the polynucleotide, and gene therapy vector that contains M3 and M5 plus an additional copy of M3 (the 5′UTR of the triple mirtron vector is set forth in SEQ ID NO: 72, see Example 28 below). In an example, described herein is a triple mirton polynucleotide, gene therapy vector, or gene therapy vector comprising a polynucleotide that expresses a rhodopsin transgene, wherein said transgene encodes functional rhodopsin, wherein the coding region of said rhodopsin transgene does not comprise the sequences of SEQ ID NOs: 5 or 7; and wherein the 5′UTR of the rhodopsin transgene consists of a 5′ end consisting of the sequence of nucleotides 1 to 423 of SEQ ID NO: 39; a 3′ end consisting of the sequence of nucleotides 424 to 439 of SEQ ID NO: 39; and a sequence inserted between nucleotides 1 to 423 and 424 to 439 of SEQ ID NO: 39; wherein said inserted sequence consists of, in a 5′ to 3′ direction: (a) SEQ ID NO: 26, (b) ATC, (c) SEQ ID NO: 37, (d) SEQ ID NO 12, and (e) SEQ ID NO: 38. In an examples, a 5′UTR of a rhodopsin transgene described herein comprises or consists of SEQ ID NO: 72. In this examples, the rhodopsin transgene coding region comprises the sequences of SEQ ID NO: 24 and SEQ ID NO: 25. In this Example, the rhodopsin transgene coding region comprises the sequence of nucleotides 1819 to 2866 of SEQ ID NO: 28. Also described herein are pharmaceutical compositions comprising the polynucleotide, gene therapy vector comprising a polynucleotide, or gene therapy vector of this example. Also described herein are methods for treating retinitis pigmentosa in a subject in need thereof, the method comprising administering to the subject the triple mirtron polynucleotide, gene therapy comprising the polynucleotide, or gene therapy vector described herein.

The disclosure will now be described in more detail, by way of example and not limitation, and by reference to the accompanying drawings. Many equivalent modifications and variations will be apparent, to those skilled in the art when given this disclosure. Accordingly, the exemplary embodiments of the disclosure set forth are considered to be illustrative and not limiting. Various changes to the described embodiments may be made without departing from the scope of the disclosure. All documents cited herein, whether supra or infra, are expressly incorporated by reference in their entirety.

The present disclosure includes the combination of the aspects and preferred features described except where such a combination is clearly impermissible or is stated to be expressly avoided. As used in this specification and the appended claims, the singular forms “a”, “an”, and “the” include plural referents unless the content clearly dictates otherwise. Thus, for example, reference to “a vector” includes two or more such entities.

Section headings are used herein for convenience only and are not to be construed as limiting in any way.

### DESCRIPTION OF THE FIGURES

FIG. 1 Schematic of rhodopsin AAV transgenes together with effective size in kb. ITR, inverted terminal repeat; RHOp, human rhodopsin promoter; Ex/Int, first exon and intron from the chicken beta actin gene together with the splice acceptor from rabbit beta globin; RHO, human rhodopsin coding sequence; WPRE, woodchuck hepatitis virus post-transcriptional regulatory element; pA, bovine growth hormone poly-adenylation sequence; ITR^{sc}, mutated inverted terminal repeat (resulting in self-complementarity).

FIGS. 2A-2B Rescue of rhodopsin expression in dissociated Nrl.GFP/Rho^{-/-} retinal cells following transduction with ssRHO, WPRE, Ex/Int and scRHO AAVs. (A) Images of cells immunostained from rhodopsin. Note GFP labelled cells stain for rhodopsin in transduced but not untransduced cells. Age-matched dissociated wild type cells are shown as a positive control in the left-hand panels. (B) Human rhodopsin mRNA levels in transduced dissociated cells measured by qPCR revealed a four-fold increase in the number of transcripts for scRHO compared with the other three vectors (**p<0.01, 1-way ANOVA).

FIGS. 3A-3B In vivo comparison of RHO-expressing AAVs. (A) Nrl.GFP/Rho^{-/-} mice received subretinal injections of the four AAV-RHO viruses. Immunostaining for rhodopsin in retinal sections suggested greatest expression with Ex/Int and SC vectors. All promoters conferred rod-specific expression. GCL ganglion cell layer; IPL inner plexiform layer; INL inner nuclear layer; OPL outer plexiform layer; ONL outer nuclear layer; IS inner segments; OS outer segments. (B) SD-OCT was performed on live mice 4 weeks post-injection. Photoreceptor layer (PRL) thickness was greatest for Ex/Int and SC-injected mice supporting the immunohistochemistry findings. *p<0.05, one-way ANOVA Holm-Sidak multiple comparison test.

FIGS. 4A-4B Western blot of protein lysates derived from the neural retina of AAV-RHO injected Nrl.GFP/Rho^{-/-} mice. (A) Example of a western blot of transduced retinal lysates stained for rhodopsin. Note that transgenic rhodopsin results in a pattern of bands similar to that derived from an untransduced wild-type mouse, albeit at a much lower level of expression. (B) Band densitometry showed that the greatest level of protein expression was achieved with Ex/Int and SC vectors. Two-way ANOVA p<0.001 for effect of vector. Tukey's multiple comparison test: scRHO vs ssRHO or WPRE, p<0.01; Ex/Int vs ssRHO or WPRE, p<0.05; ssRHO vs WPRE, ns; Ex/Int vs SC, ns.

FIGS. 5A-5C Western blots showing overexpression of rhodopsin following subretinal injection of Ex/Int and scRHO vectors. (A) Example western blot showing increased rhodopsin band density in injected right eyes (OD) compared with uninjected left eyes (OS). (B, C) Band densitometry normalised to beta actin. Two-way ANOVA p=0.21 for effect of SC, p=0.023 for effect of Ex/Int.

FIG. 6 Intensity response curves derived from dark-adapted electroretinography (ERG) recordings from P23H mice injected unilaterally with AAV-Ex/Int. No difference in responses between eyes was detected in the intermediate dose group at either one or three months post-injection, and no slowing of the degeneration was observed. The high dose of vector appeared toxic.

FIG. 7 GFP-Mirtron plasmid maps. The 76 bp mirtron insert at the BstB1 restriction site disrupts the GFP coding sequence and is predicted to cause a frame shift in GFP if not correctly and completely spliced.

FIG. 8 Transfection of HEK293 cells with GFP-Mirtron plasmids. Differing levels of green fluorescence indicate variation of splicing efficiency between mirtron designs.

FIGS. 9A-9B Splicing efficiency of mirtrons 1-7. (A) Normalized fluorescence assay based on signal from the lysates of transfected cells shown in FIG. 8. (B) PCR was performed from GFP-mirtron transfected HEK293 cDNA with mirtron-spanning primers. Relative densitometry readings from PCR amplicon bands shown in (B) (data not shown).

FIGS. 10A-10B (A) The plasmid and (B) Luciferase assay (Promega).

FIGS. 11A-11B Rhodopsin knockdown efficiency of mirtrons 1-7 measured with the Luciferase assay against (A) human and (B) mouse coding sequences. ****p<0.0001, one-way ANOVA Dunnett's multiple comparison test.

FIGS. 12A-12B Both splicing and a specific target sequence are required to achieve mirtron-mediated rhodopsin knockdown.

FIG. 13 mRNA target context influences potency of mirtrons. ‘Full RHO’ corresponds to the vector into which the full length human rhodopsin coding sequence has been cloned. The ‘Short RHO’ plasmid contains just the region of the human rhodopsin sequence corresponding to the 21 bp M3 target sequence with 25 bp of 5′ and 3′ flanking DNA. ****p<0.0001, 2-way ANOVA, Sidak's multiple comparison test.

FIG. 14 RHO-GFP traffics to the plasma membrane.

HEK293 cells were transfected with the CAG.RHO-GFP. WPRE plasmid and stained for rhodopsin with the N-terminus directed antibody 4D2.

FIG. 15 RHO-Luc traffics to the plasma membrane. HEK293 cells were transfected with the RHO plasmid (designed for the conventional dual luciferase assay; top row), or the RHO-Luc plasmid (designed for the dual luciferase fusion assay; bottom row). Schematics of the corresponding proteins are shown to the left.

FIG. 16 Mirtron-mediated rhodopsin knock-down using the Luciferase assay in conjunction with RHO (‘standard assay’) and RHO-Luc (‘Fusion Assay’). Note that knock-down effect is similar when measured by the two assays in all cases except for that of Mirtron 2.

FIG. 17 Predicted ‘off targets’ are not subject to mirtron-mediated knock-down

FIGS. 18A-18B ESE-rich mirtron-flanking eGFP CDS sequences and ESEs. A: 5′ sequence. B: 3′ sequence. Distances upstream from splice donor motif or downstream of splice acceptor motif of mirtron are indicated.

FIG. 19 Experimental design to test the efficacy of 5′-UTR mirtrons and their effect on downstream gene expression.

FIGS. 20A-20C Mirtrons are more effective when located in the 5′-UTR rather than the CDS of GFP. Dual luciferase assays were performed comparing M2 with M2-UTR (A), M3 with M3-UTR (B), and M5 with M5-UTR (C). n=6 in all cases. ns, not significant; *p<0.05; ***p<0.001; ****p<0.0001; Two-way ANOVA with correction for multiple comparisons.

FIGS. 21A-21B Enhanced splicing may explain improved efficacy of 5′-UTR mirtrons. (A) PCR splice product analysis for nested and 5′-UTR mirtrons. (B) band densitometry. n=3, **p<0.01, Two-way ANOVA Sidak's multiple comparison test.

FIGS. 22A-22B Correct splicing of mirtrons 3 and 5 from the 5′-UTR is confirmed by Sanger sequencing of amplified cDNA from transfected HEK293 cells.

FIGS. 23A-23D Mirtrons in tandem result in increased rhodopsin knock-down. (A, B) Two copies of M3 in series within the 5′-UTR is more effective than one. (C, D) One copy of M3 and one copy of M5 in series is more effective than either mirtron alone in the 5′-UTR. Data shown is for the version of M5 (M5H and M5M) directed against its corresponding species. ns=not significant; *p<0.05; **p<0.01; ****p<0.0001; 2-way ANOVA Sidak's multiple comparison test.

FIG. 24 Splice analysis of tandem 5′-UTR mirtrons. Note that no bands corresponding to those predicted for unspliced (412 bp), single spliced (336 bp) or exon-skipped (166 bp) transcripts were detected.

FIGS. 25A-25B Splice analysis of tandem 5′-UTR mirtrons. Sanger sequencing confirmed independent and precise splicing of tandem mirtrons in the 5′-UTR of GFP.

FIG. 26 Mirtrons located within the 5′-UTR of GFP effectively knock down the RHO-Luciferase fusion protein. *p<0.05; ***p<0.001 Two-way ANOVA Sidak's multiple comparison test.

FIG. 27 Reporter gene expression of 5′-UTR mirtrons. HEK293 cells transfected with 5′-UTR mirtron-GFP constructs were compared with that of CAG.GFP.WPRE. n=6; ****p<0.0001, one-way ANOVA Dunnett's multiple comparisons test.

FIG. 28 M3/M5H-UTR achieves >75% knock-down of both human and mouse rhodopsin.

FIG. 29 Codon modification of rhodopsin confers resistance to mirtrons

FIGS. 30A-30B The RHO^{M3/5R} sequence leads to expression of rhodopsin in vitro. (A) Rhodopsin protein translated from both RHO and RHO^{M3/5R} is recognised by an N-terminus specific monoclonal antibody (4D2, Abcam), a C-terminus specific antibody (1D4, Abcam) and a polyclonal antibody (ab3424, Abcam). (B) Rhodopsin traffics to the plasma membrane in both instances.

FIG. 31 RHO^{M3/5R} is capable of driving the rod-derived electroretinogram. Dark-adapted intensity-response curves for a- and b-waves derived from injected right and uninjected left eyes of Rho^{-/-} mice four weeks post injection. Two-way ANOVA for effect of injection p=0.0022 for a-wave and p=0.0043 for b-wave.

FIG. 32 Rhodopsin translated from the RHO^{M3/M5R} transcript traffics to outer segments. Nrl.GFP/Rho^{-/-} mice (in which rods are labelled with GFP) received unilateral subretinal injections of AAV2/8^{Y733F}.RHOp.Ex/Int.M3/M5.RHO^{M3/5R}.WPRE. Four weeks later, animals were sacrificed and retinal sections immunostained for rhodopsin.

FIG. 33 Genome of the ‘block’ and replace' AAV vector

FIGS. 34A-34B cDNA derived from AAV-injected Rho^{P23H/+} retinas at low dose (2×10⁸ gc) and high dose (2×10⁹ gc) was used as template for PCR with primers spanning the mirtron regions of the transcript. The gel above shows that the primary PCR product corresponds in size to that expected from transcripts where both mirtrons are individually spliced out: >90% based on band densitometry (left).

FIG. 35 mRNA analysis: Mirtron 3 induces knock-down of mouse rhodopsin in vivo. No significant reduction in mRho was noted at low or high dose after delivery of AAV-Ex/Int which contains no mirtrons. When the mirtron-containing vector was delivered at high dose, a 34% knockdown in total retinal mRho mRNA was evident compared with fellow sham-injected eyes P=0.0024, 2-way ANOVA Sidak's multiple comparison test. No significant knockdown was seen at low dose.

FIG. 36 mRNA analysis continued: human RHO supplementation levels after delivery of AAV-M3/5^{H}.RHO^{M3/5R}.WPRE. ‘Replacement’ with human RHO expressed as a fold change relative to native mouse Rho levels in fellow sham-injected eyes. At low dose, RHO supplementation approximates to native levels. At high dose, a 4.6 fold increase is seen, (P=0.0011, 2-way ANOVA Sidak's multiple comparison test) suggesting a degree of overexpression.

FIG. 37 mRNA analysis continued. Human RHO expression correlates with mRho knockdown in eyes treated with the mirtron-containing vector (right) but not with the AAV without Mirtron 3 (left). Together, this gene expression analysis represents the first demonstration of function of an artificial mirtron in vivo.

FIG. 38 Relative rescue of photoreceptor layer thickness (yellow arrows) in P23H eyes injected with 2×10⁸ gc of AAV-M3/5.RHO. Representative SD-OCT images taken along the horizontal meridian in a treated mouse. PRL thickness was greater in injected than in uninjected eyes in the temporal and nasal retina (both p<0.0001 2-way ANOVA, Sidak's multiple comparison test). No significant difference was detected for superior retinal locations (likely due to the effect of retinal detachment) or inferior locations (likely due to insufficient dose).

FIG. 39 Early evidence that subretinal delivery of low dose (LD: 2×10⁸ gc) AAV-M3/5.RHO leads to relative preservation of the photoreceptor layer (PRL) as measured in vivo by SD-OCT. The ratio of PRL thickness in injected versus uninjected eyes is shown as a function of retinal location for LD and sham-injected groups 1 month post-injection. Note increased retinal thickness (ratio>1) in LD but not in sham-injected eyes along the horizontal meridian (nasal and temporal retina).

FIG. 40 OCT analysis looking for treatment effect. OCT provides non-invasive in vivo cross-sectional imaging of the retina. For all cohorts, right eyes were injected and left eyes were uninjected as controls. A small but significant positive effect on overall photoreceptor layer (PRL) thickness was seen in the low dose cohort whilst a small but significant damaging effect was seen for the sham group (attributable to surgery). No significant change in overall mean PRL thickness was seen in the high dose injected eyes. Vector=AAV.RHOp.Ex/Int.M3.M5^{H}.RHO^{M3/5R} WPRE

FIG. 41 OCT analysis for the three groups by retinal location following superior subretinal injection. Vector=AAV.RHOp.Ex/Int.M3.M5^{H}.RHO^{M3/5R}.WPRE

FIG. 42 OCT analysis with AAV-Ex/Int vector. Data shown is 1 and 2 months post-injection. Compared with equivalent data from vector with mirtrons (FIG. 7), no benefit is seen at low dose and high dose appears more damaging.

FIG. 43 OCT analysis for the three groups by retinal location following superior subretinal injection. Again, compared with FIG. 8 (data for vector with mirtrons), no benefit seen at low dose and high dose appears more damaging. Vector=AAV.RHOp.Ex/Int. RHO.WPRE

FIG. 44 Dark-adapted ERG analysis. ERG measures electrical signal from the retina, a-wave is from photoreceptors, b-wave from inner retina. Small benefit seen at low dose, whilst high dose and sham seem to have small deleterious effects.

FIG. 45 Light-adapted ERG analysis. Represents signal from cone photoreceptors. Small benefit seen at low dose, whilst high dose and sham seem to have deleterious effects, more so in high dose group.

FIG. 46 One month dark- and light-adapted ERG analysis following delivery of AAV.RHOp.Ex/Int. RHO.WPRE.

FIG. 47 New AAV vectors without the WPRE.

FIGS. 48A-48C RT-qPCR analysis identified simultaneous changes in expression of endogenous and codon-modified rhodopsin in wild-type retinal organoids following treatment with double and triple mirtron vectors. (FIG. 48A) Endogenous rhodopsin transcript levels were reduced in double and triple mirtron vector treated wild-type retinal organoids. In the triple mirtron treated samples, no endogenous rhodopsin was detected. (FIG. 48B) Levels of replacement codon-modified rhodopsin were similar in double and triple mirtron vector treated wild-type retinal organoids. (C) Rhodopsin (RHO) protein staining was comparable in untreated wild-type retinal organoids and triple mirtron vector treated wild-type retinal organoids. Recoverin (REC) is a marker for photoreceptors.

FIG. 49 Bulk RNAseq revealed transcriptome changes in untreated and treated P23H retinal organoids. Differential gene expression analysis of important pathways identified greater changes were achieved in triple mirtron vector treated P23H retinal organoids. Dotted lines indicate significance threshold (fold change >2, p-value <0.05). Changes are relative to untreated P23H samples.

FIGS. 50A-50B Rho^{+/-,P23H+} mice received either double or triple mirtron vector at different doses (high=1E+09 genome copies/eye, medium=5E+08 gc/eye and low=1E+08 gc/eye). (FIG. 50A) Endogenous rhodopsin transcripts were knocked down by both the double and triple mirtron vector with codon-modified rhodopsin detected at dose-dependent levels. (FIG. 50B) Photoreceptor layer thickness measurements were significantly improved in the inferior retina of triple mirtron vector treated eyes with some toxicity evident in the superior retina of the high dose cohort.

FIGS. 51A-51C. Triple mirtron vector preserves the retinal structure and function in Rho^{+/-,P23H+} mice. (FIG. 51A) Photoreceptor layer thickness measurements were consistently greater in both the superior and inferior retina of treated eyes compared to paired uninjected eyes. (FIG. 51B) Scotopic ERG b-wave responses were greater in treated eyes compared to paired uninjected eyes. (FIG. 51C) Significant knockdown of endogenous rhodopsin transcripts was detected by qPCR in triple mirtron injected eyes with expression of mirtron-resistant codon-modified rhodopsin also confirmed.

FIGS. 52A-52C Triple mirtron vector preserves the retinal structure and function in Rho^{+/C110R} mice. (FIG. 52A) Photoreceptor layer thickness measurements were consistently greater in both the superior and inferior retina of treated eyes compared to paired uninjected eyes. (FIG. 52B) Scotopic ERG b-wave responses were greater in treated eyes compared to paired uninjected eyes. (FIG. 52C) Significant knockdown of endogenous rhodopsin transcripts was detected by qPCR in triple mirtron injected eyes with expression of mirtron-resistant codon-modified rhodopsin also confirmed.

FIGS. 53A-53B Triple mirtron vector injection in wild-type mice caused no adverse effects. (FIG. 53A) Endogenous rhodopsin was suppressed with expression of replacement codon-modified rhodopsin detected. (FIG. 53B) Triple mirtron vector treated eyes suffered no negative impact on function compared to paired sham injected eyes.

### DESCRIPTION OF THE SEQUENCES

SEQ ID NOs: 1, 2 set forth human rhodopsin CDS and amino acid sequences.
SEQ ID NOs: 3 to 9 set forth target sequences in rhodopsin gene of mirtrons 1 to 7
SEQ ID NOs 10 to 16 set forth the polynucleotide sequences of mirtrons 1 to 7.
SEQ ID NOs 17 to 23 set forth the guide strand sequences of mirtrons 1 to 7.
SEQ ID NOs: 24, 25 set forth modified target sequences that are resistant to mirtrons 3, 5.
SEQ ID NO: 26 sets forth mirtrons 3 and 5 with 5′, 3′ and intervening ESE-rich sequences derived from eGFP CDS.
SEQ ID NO: 27 sets forth the 5′UTR sequence of vector
AAV2/8^{Y733F}.RHOp.Ex/Int.M3/M5.RHO^{M3/5R}.WPRE.
SEQ ID NO: 28 sets forth the ITRs and intervening transgene sequence of vector
AAV2/8^{Y733F}. RHOp.Ex/Int.M3/M5.RHO^{M3/5R}.WPRE.
SEQ ID NO: 29 sets forth the WPRE sequence.
SEQ ID NO: 30 sets forth the BstB1 restriction site and adjacent 3′ ‘G’
SEQ ID NO: 31 sets forth the polynucleotide sequence of vector
AAV2/8^{Y733F}. RHOp.Ex/Int.M3/M5.RHO^{M3/5R}.WPRE
SEQ ID NOs: 32 and 33 set forth AAV2 forward and reverse ITR sequences
SEQ ID NO: 34 sets forth the sequence of the human rhodopsin promoter
SEQ ID NOs: 35, 36 set forth eGFP CDS and amino acid sequences.
SEQ ID NO: 37, 38 set forth ESE-rich 5′ and 3′ mirtron flanking sequences
SEQ ID NO: 39 sets forth a 5′UTR sequence without mirtrons
SEQ ID NO: 40 sets forth an ESE-rich sequence
SEQ ID NOs: 41 to 47 set forth forward oligonucleotide sequences for mirtrons 1- to 7
SEQ ID NOs: 48 to 54 set forth reverse oligonucleotide sequences for mirtrons 1- to 7
SEQ ID Nos: 55 to 57 set forth mirtron, forward oligonucleotide and reverse oligonucleotide sequences for mirtron M5M
SEQ ID Nos: 58 to 60 set forth mirtron, forward oligonucleotide and reverse oligonucleotide sequences for mirtron M2U-M2.
SEQ ID Nos: 61 to 63 set forth mirtron, forward oligonucleotide and reverse oligonucleotide sequences for mirtronScr-M2.
SEQ ID NO: 64 sets forth the polynucleotide sequence of vector AAV2/8^{Y733F}.RHOp.Ex/Int.M3/M5.RHO^{M3/5R}
SEQ ID NO: 65 sets forth the polynucleotide sequence of vector AAV2/8^{Y733F}.RHOp.Ex/Int.M3/M5M.RHO^{M3/5R}
SEQ ID NO: 72 sets forth a triple mirtron vector which contains M3 and M5 plus an additional copy of M3, where the 5′UTR of the rhodopsin transgene consists of a 5′ end consisting of the sequence of nucleotides 1 to 423 of SEQ ID NO: 39; a 3′ end consisting of the sequence of nucleotides 424 to 439 of SEQ ID NO: 39; and a sequence inserted between nucleotides 1 to 423 and 424 to 439 of SEQ ID NO: 39; where the inserted sequence consists of, in a 5′ to 3′ direction: (a) SEQ ID NO: 26, (b) ATC, (c) SEQ ID NO: 37, (d) SEQ ID NO 12, and (e) SEQ ID NO: 38.

### DETAILED DESCRIPTION

### Mirtrons

The invention is concerned with the design, preparation and use of vectors that comprise mirtrons to knock down the expression of a gene. Such a gene may be referred to herein as a target gene or the gene targeted by the mirtron. A mirtron can knock down expression of a target gene if the polynucleotide sequence of the guide strand of the mirtron complements (“recognises”) a polynucleotide sequence that is present in the target gene. The polynucleotide sequence in the target gene that complements the guide strand of the mirtron may be referred to herein as a target sequence.

In some cases in accordance with the present invention the vector is a gene therapy vector. The target gene may be any gene that is expressed in a subject where the subject would receive therapeutic benefit from a reduction in the expression of the gene, either alone or in combination with other therapeutic measures, such as co-expression of a transgene as discussed below.

A mirtron is a hairpin intron that is spliced out and functions as a microRNA. Mirtrons may be capable of inhibiting gene expression of a target mRNA through the process of RNA interference (RNAi). The term “mirtron” as referred to herein includes classical mirtrons, in which the 5′ and 3′ splice sites are located near the base of the hairpin, and 5′ or 3′ tailed mirtrons, which may be further processed by exonuclease digestion after splicing.

Considerations that may be taken into account when designing artificial mirtrons have been described previously in, for example, Seow et al. (RNA (2012) 18:1328-1337) and Kock et al. (Nucleic Acids Research (2015).

Each mirtron begins with the splice donor motif (‘GT’) and ends with the splice acceptor motif (‘AG’). The stem of the hairpin comprises a guide strand and a complementary passenger strand. The guide strand is designed to recognise a target mRNA sequence by complementary Watson-Crick base pairing. Mismatches may be introduced at the base of the hairpin in the passenger strand to facilitate correct strand selection by the RNA-induced silencing complex (RISC), as for examples described in Schwarz et al. (2003, Cell 115(2): 199-208). To maintain efficacy the guide strand complements the target sequence.

The guide strand may be typically at least 15, 16, 17, 18, 19, 20 or 21 nucleotides in length, and up to 21, 22, 23, 24, 25, 30, 35, 40, 45, 50 or more nucleotides in length. In some cases the guide strand is between 18 and 23 nucleotides in length or 21 nucleotides in length. The guide strand may in some cases be less than 20, 15, 14, 13, 12, 11, 10, 9, 8, 7, 6, 5, 4, 3, 2 or 1 nucleotides from the 5′ splice donor motif. The guide strand and/or the passenger strand may comprise a T or more typically an A at position 21. The guide strand and/or the passenger strand may comprise an A position 5. The guide strand and/or the passenger strand may comprise three or more A's or T's in the last 5 nucleotides. The guide strand and/or the passenger strand may comprise an A, C or T at position 15. The guide strand and/or the passenger strand may comprise an A position 8.

In between the guide strand and the passenger strand is a hairpin loop. The hairpin loop may be typically 5, 6, 7, 8 or 9 to 9, 10, 11, 12, 13, 14, 15, 20, 25, 30, 35 or more nucleotides in length. In some cases the hairpin loop is 8 to 12 nucleotides in length. In some cases the hairpin loop is 9 nucleotides in length and/or comprises the sequence TTCAAGAGA.

The lariat branch point is located either in the hairpin loop (classical and 5′ tailed mirtrons) or near the 3′ end of the hairpin structure, typically within 1, 2, 3, 4, 5, 6, 7, 8, 9, 10, 11, 12, 13, 14 or 15 nucleotides 5′ or 3′ of the end of the hairpin (3′ tailed mirtrons). In 3′ tailed mirtrons the hairpin may in some cases end within 3, within 2 or within 1 nucleotide downstream of the lariat branch point.

Downstream of the hairpin is a polypyrimidine tract and an AG splice acceptor motif. The polypyrimidine tract may typically comprise at least six uninterrupted polypyrimidines. Further, the polypyrimidine tract may typically comprises a stretch of at least 10 polypyrimidines with at most one purine interruption, or a stretch of at least 13 polypyrimidines with at most two purine interruptions, or a stretch of at least 16 polypyrimidines with at most three purine interruptions.

At a molecular level, a mirtron-based gene therapy strategy offers a number of advantages over other gene knock-down or ‘block and replace’ approaches taken previously:
a. The mirtron released after splicing is precisely defined by the splice donor and acceptor mRNA sequences which means that the resulting short hairpin RNA is likely to be associated with fewer off-target effects that may arise following aberrant miRNA processing from PolIII class promoters.
b. Mirtron processing is Drosha-independent which may reduce the likelihood of toxicity related to miRNA pathway saturation.
c. As intronic sequences, mirtrons may be co-expressed together with a ‘hardened’ transgene transcript under the same PolII promoter. This allows for perfect matching of ‘blockage’ and ‘replacement’ and avoids any imbalances that may result when different promoters or vectors are used for the knock-down and replacement arms of the treatment, strategies employed by other researchers. Indeed, using separate promoters may in fact make a disease process worse if the promoter driving the replacement transcript were to be silenced with time.

### 5′UTR and Exonic Splice Enhancers (ESEs)

Exonic splice enhancers (ESEs) are certain 6 base DNA sequence motifs within an exon that are known in the art to be capable of directing or enhancing accurate and efficient splicing out of an intron. It has been suggested in the art to select an insertion site for a mirtron in the coding region of a transgene that is rich in ESE's to promote efficient splicing out of a mirtron.

According to the present invention, it is advantageous to locate a mirtron in the 5′UTR of a transgene for co-expression with the mirtron. The inventors reasoned that by placing a mirtron upstream of a transgene start codon, expression of the transgene should be less disrupted than if the mirtron is located in the CDS. There is no risk of mutant protein production (provided that no ATG motifs are introduced into the 5′UTR that could act as alternative start codons) as the transgene CDS is not interrupted. Location of the mirtron in the 5′ UTR should also not increase nonsense mediated decay of the transgenic transcript, which may occur if the mirtron is located in the 3′UTR.

Furthermore, the inventors realised that by locating the mirtron in the 5′UTR it becomes unnecessary to rely upon ESEs that occur naturally in the CDS, as has been proposed previously, or that could perhaps be engineered using alternative codons into the CDS without disrupting expression of the transgene. Instead additional ESEs as described below may be introduced into the 5′UTR to promote or maintain efficient mirtron splicing. The 5′UTR of a transgene may be modified by the insertion of both a mirtron and one or more additional or exogenous sequences comprising one or more ESEs, and that are not part of the un-modified consecutive sequence of the 5′UTR. In other cases a mirtron may be inserted into a transgene comprising a 5′UTR that comprises one or more ESEs.

The additional sequence(s) or ESEs may be upstream and/or downstream of the mirtron in the modified 5′UTR. The 5′UTR and/or the additional or exogenous sequence may comprise (at least) 1, 2, 3, 4, 5, 6, 7, 8, 9, 10, 11, 12 or more ESEs. The one or more ESE(s) may be fully within 6, 7, 8, 9, 10, 11, 12, 13, 14, 15, 16, 17, 18, 19, 20, 25, 30, 35, 40, 45, 50, 55, 60, 80 or 100 nucleotides 5′ of the splice donor motif and/or 3′ of the splice acceptor motif of the mirtron. ESEs may be identified using the RESCUE-ESE online tool (http://genes.mit.edu/burgelab/rescue-ese/). An ESE may have any one of the sequences listed in Table 1.

TABLE 1
ESE sequences

AAAACC AAGATC AGAAGA ATGCAA GAAACA GAGGAT TGAAGA
ACTGGA AAAAGA AAGATG AGAAGC ATGGAA GAAACC GATATC
TGAAGC AGTGAC AAAAGC AAGCAA AGAAGG ATGGCG GAAACG
GATATG TGAAGG ATCTTC AAACAG AAGCAG AGAAGT ATTCAG
GAAACT GATCAA TGAAGT ATGAAA AAACCA AAGCCA AGAATG
ATTGGA GAAAGA GATCAT TGAGAA ATGGAT AAACCT AAGCTA
AGACAA CAAAAC GAAAGC GATGAA TGATGA ATCCTC AAACGA
AAGGAA AGACAT CAAAAG GAAATC GATGAG TGCAAC CAAACA
AAAGAA AAGGAC AGACGA CAAAGA GAACAA GATGAT TGGAAA
CAGATC AAAGAC AAGGAT AGAGAA CAACTT GAACAT GATGCA
TGGAAG CATCAG AAAGAG AATCAA AGAGAT CAAGAA GAACTG
GATGGA TGGAAT CGAATG AAAGAT AATCCA AGAGGA CAAGAT
GAACTT GATTCA TGGATC CGTCGC AAAGCA AATGAC AGATGA
CAAGTA GAAGAA GCAAAA TTCAGA CTACAT AAAGCT AATGGA
AGATGC CAATCA GAAGAC GCAAGA TTCGAA CTCCAT AAAGGA
ACAAAG AGATGT CAGAAA GAAGAG GCAGAA TTGAAG GAAAAT
AAATCC ACAACG AGCAAA CAGAAG GAAGAT GGAAAA TTGCGA
GAACCA AACAAC ACAACT AGCAGA CAGAAT GAAGCA GGAAAC
TTGGAA GCGAAT AACAAG ACAAGA AGGAAA CAGAGG GAAGGA
GGAAGA TTGGAT GGAGAT AACAGA ACAGAA AGGAAC CAGGAA
GAAGTA GGAGAA TTTGGA GTCGAC AACCAA ACCTGA AGGAAG
CCTGAA GAAGTT GGAGGA AAAAAG GTGTCG AACGAA ACGAAA
AGGACA CGAAAA GAATCA GGATCA AAACTC GTTGGA AACTGG
ACGAAG AGGAGA CGAACA GACAAA GTCAAG AACATG TATGAA
AACTTC ACGACT AGTGAA CGAAGA GACAAT GTGAAG AACCAG
TCAACG AAGAAA ACTGAA ATCAAA CGACGA GACGAA TACAAG
AACTAC TCATCA AAGAAC ACTTCA ATCAAG CGTATG GACGAC
TACAGA AAGGAG TCGTCG AAGAAG ACTTCG ATCAAT CTGAAA
GAGAAA TATGGA AATACG TCTTCA AAGAAT AGAAAA ATCAGA
CTGAAG GAGAAG TCAAGA AATCAG TGACTG AAGACA AGAAAC
ATCCAA CTTCAG GAGAGA TCAGAA AATGAA TGGAAC AAGACT
AGAAAG ATGAAG GAAAAA GAGATG TCAGGA ACATGA TGTGGA
AAGAGA AGAACA ATGAGA GAAAAC GAGGAA TGAAAC ACGCAA
AAGAGG AGAACT ATGATG GAAAAG GAGGAG TGAAAG ACTACA

In some cases the mirtron is flanked at the 5′ end by a polynucleotide sequence that comprises an ESE at any or the first 1, 2, 3, 4, 5, 6, 7 or all 8 of the following positions upstream of the splice donor motif of the mirtron:
1 nucleotide upstream, optionally a TTCGAA motif;
7 nucleotides upstream, optionally a GTCAAG motif;
11 nucleotides upstream, optionally a TGAAGT motif;
12 nucleotides upstream, optionally a CTGAAG motif;
24 nucleotides upstream, optionally a ACAAGA motif;
25 nucleotides upstream, optionally a TACAAG motif;
27 nucleotides upstream, optionally a ACTACA motif;
28 nucleotides upstream, optionally a AACTAC motif.

In some cases the mirtron is flanked at the 3′ end by a polynucleotide sequence that comprises an ESE at any or the first 1, 2, 3, 4 or all 5 of the following positions downstream of the splice acceptor motif of the mirtron:
27 nucleotide downstream, optionally a CTGAAG motif;
28 nucleotides downstream, optionally a TGAAGG motif;
46 nucleotides downstream, optionally a AAGGAG motif;
47 nucleotides downstream, optionally a GAGGAT motif;
48 nucleotides downstream, optionally a GGAGGA motif.

### Mirtron Insertion Site

When designing and/or cloning a plasmid or vector for expressing a mirtron it is usually beneficial to maximise the efficiency and accuracy of splicing out of the mirtron. It may also be beneficial to test the efficiency and accuracy of splicing using a reporter gene as described below. The following may be taken into account when choosing a location for insertion of a mirtron in the 5′UTR of a transgene for co-expression with the mirtron or in the CDS of a reporter gene or a fragment thereof.

It is beneficial to choose a position that is rich in ESEs to facilitate splicing. A ‘MAG’ (where ‘M’=‘A’ or ‘C’) motif at the end of the 5′ exon and a ‘G’ at the beginning of the 3′ exon is usually required for an intron to be recognised (as well as the splice donor and acceptor motifs within the intron/mirtron itself). Therefore a suitable insertion site could be identified by searching for a ‘MAGG’ motif in an ESE rich region of a 5′UTR for a transgene for co-expression with the mirtron or the CDS of a suitable reporter gene. The region should ideally lack any ‘ATG’ translation start motifs.

A mirtron may be cloned into the selected site using appropriate restriction enzymes or any other suitable alternative approach known in the art, such as overlap extension PCR. Particularly advantageous is to identify a BstB1 restriction site in a suitable region of the 5′UTR or reporter gene CDS. The BstB1 restriction site is preferably followed by a ‘G’ residue (‘TTCGAAG’). The last three bases ‘AAG’ provides the ‘MAG’ motif. The mirtron, beginning with the splice donor motif ‘GT’, is inserted immediately downstream. A mirtron having appropriate “sticky ends” that complement those made by cutting with BstB1 can be made, for example by annealing complementary oligonucleotides having sequence overhangs at the 5′ and 3′ ends. The forward oligonucleotide has an 5′ ‘CGAAG’ motif compared to the sequence of the desired mirtron and correspondingly omits a ‘CGAAG’ motif at the 3′ end. This includes the ‘AG’ splice acceptor motif designed to be at the 3′ end of the mirtron once inserted. The reverse oligonucleotide omits a ‘CTT’ motif at the 5′ end compared to the reverse complement of the desired mirtron, and correspondingly includes an additional ‘CTT’ at the 3′ end. By taking this approach, the mirtrons can be inserted at the BstB1 restriction site in such a way that the gene (for example a reporter gene) is reformed when the mirtron is spliced out.

A similar approach could in principal be applied using other suitable restriction enzymes. Suitable restriction enzymes may also be used to excise a suitable fragment of a reporter gene CDS and adjacent or embedded mirtron(s) from a plasmid or vector for transfer to a new location in the 5′UTR of a transgene. In other cases a fragment may be amplified, for example by PCR amplification. Alternatively any other suitable cloning method may be used as are well known in the art.

### Reporter Genes

When designing a new mirtron, or a plasmid or vector for expressing a mirtron, it will often be advantageous to test the efficacy and accuracy of splicing out of the mirtron using a reporter gene. For example, in some cases the mirtron will be inserted into the coding sequence of the reporter gene such as to produce a frame-shift that will prevent expression of the reporter gene unless the mirtron is properly spliced out from the reporter transcript.

The inventors reasoned that the splicing characteristics that are observed when a mirtron is embedded in the CDS of a reporter gene may be maximally and conveniently maintained when the mirtron is transferred to a new location, such as the 5′UTR of a transgene for co-expression with the mirtron, if the mirtron is flanked in the new location by the same 5′ and/or 3′ flanking sequence from the reporter gene CDS. Hence according to the present invention a mirtron may be inserted in the 5′UTR of a transgene for co-expression and flanked at the 5′ and/or 3′ end by a fragment of a reporter gene CDS.

In some cases the mirtron may be embedded in a fragment of the reporter gene CDS with sequences 5′ and 3′ of the mirtron that are continuous with each other in the CDS of the reporter gene when uninterrupted by insertion of the mirtron. In some cases a short linker may be present between the mirtron and a 5′ and/or 3′ flanking reporter gene CDS fragment. Such a linker may be typically up to 1, 2, 3, 4 or 5 nt long. The 5′ and/or 3′ reporter gene CDS fragment(s) may comprise one or more ESEs as described herein. However, 5′ and/or 3′ reporter gene CDS fragment(s) will typically not comprise any ‘ATG’ motifs that could act as an alternative translation start site.

In some cases the mirtron may be flanked at the 5′ end by a fragment of a GFP (green fluorescent protein) or eGFP (enhanced GFP) CDS. The fragment may end at position 346 of the CDS when aligned with the eGFP sequence of SEQ ID NO: 35. In some cases the mirtron may be flanked at the 3′ end by a fragment of a GFP or eGFP CDS. The fragment may end at position 346 of the CDS when aligned with the eGFP sequence of SEQ ID NO: 35. The fragment may be a fragment of an eGFP CDS having the polynucleotide sequence of SEQ ID NO: 35 or a variant thereof having at least 80%, 85%, 90%, 95%, 98% 99% or 100% sequence identity to the polynucleotide sequence of SEQ ID NO: 35.

In some cases the reporter gene CDS fragment comprises an ESE at any or the first 1, 2, 3, 4, 5, 6, 7 or all 8 of the following positions when the CDS is aligned with the eGFP sequence of SEQ ID NO: 35 and/or at the following position relative to the splice donor motif of the mirtron:
positions 340-345 and/or 1 nucleotide upstream, optionally a TTCGAA motif;
positions 334-339 and/or 7 nucleotides upstream, optionally a GTCAAG motif;
positions 330-335 and/or 11 nucleotides upstream, optionally a TGAAGT motif;
positions 329-334 and/or 12 nucleotides upstream, optionally a CTGAAG motif;
positions 317-322 and/or 24 nucleotides upstream, optionally a ACAAGA motif;
positions 316-321 and/or 25 nucleotides upstream, optionally a TTCGAA motif;
positions 314-319 and/or 27 nucleotides upstream, optionally a TTCGAA motif;
positions 313-318 and/or 28 nucleotides upstream, optionally a TTCGAA motif.

In some cases the reporter gene CDS fragment comprises an ESE at any or the first 1, 2, 3, 4 or all 5 of the following positions in an eGFP sequence aligned with SEQ ID NO: 35 and/or at the following position relative to the splice acceptor motif of the mirtron: positions 373-378 and/or 27 nucleotide downstream, optionally a CTGAAG motif; positions 374-379 and/or 28 nucleotides downstream, optionally a TGAAGG motif; positions 391-396 and/or 46 nucleotides downstream, optionally a AAGGAG motif; positions 329-334 and/or 47 nucleotides downstream, optionally a GAGGAT motif; positions 317-322 and/or 48 nucleotides downstream, optionally a GGAGGA motif.

In some cases the reporter gene CDS fragment 5′ of the mirtron comprises or consists of the nucleotide sequence of SEQ ID NO: 37, or a fragment thereof comprising one or more ESEs as described above, or a sequence having at least 80%, 82%, 84%, 86%, 88%, 90%, 92%, 94%, 96%, 98%, 99% or 100% sequence identity to SEQ ID NO: 37. In some cases the reporter gene CDS fragment 3′ of the mirtron comprises or consists of the nucleotide sequence of SEQ ID NO: 38, or a fragment thereof comprising one or more ESEs as described above, or a sequence having at least 80%, 82%, 84%, 86%, 88%, 90%, 92%, 94%, 96%, 98%, 99% or 100% sequence identity to SEQ ID NO: 38.

In some cases the mirtron, or two or more mirtrons and optionally any intervening sequence between the two or more mirtrons, are embedded between positions 423 and 424 of a 5′UTR sequence aligned with the polynucleotide sequence of SEQ ID NO: 39. The 5′UTR sequence, excluding the embedded mirtron and any intervening sequences, may have at least 80%, 85%, 90%, 95%, 98% 99% or 100% sequence identity with the polynucleotide sequence of SEQ ID NO: 39. The sequence between any two mirtrons in the 5′UTR may have the polynucleotide sequence of SEQ ID NO: 40, or a polynucleotide sequence having at least 80%, 85%, 90%, 95%, 98% 99% or 100% sequence identity with the polynucleotide sequence of SEQ ID NO: 40 Any of the sequence set out above may comprise one or more of the ESEs in the positions discussed above.

Homology or sequence identity as referred to anywhere herein can be measured using known methods. For example the UWGCG Package provides the BESTFIT program which can be used to calculate homology (for example used on its default settings) (Devereux et al (1984) Nucleic Acids Research 12, 387-395). The PILEUP and BLAST algorithms can be used to calculate homology or line up sequences (typically on their default settings), for example as described in Altschul S. F. (1993) J Mol Evol 36:290-300; Altschul, S, F et al (1990) J Mol Biol 215:403-10. Software for performing BLAST analyses is publicly available through the National Center for Biotechnology Information (http://www.ncbi.nlm.nih.gov/).

A “reporter gene” as used herein is any gene the expression of which is readily detected and quantified, for example by such means as detecting a reaction catalysed by a reporter gene product or the induction of visually identifiable characteristics such as emitted light or fluorescence. Examples of reporter genes include genes for fluorescent proteins (for example GFP, sGFP, RFP, dsRed, mCherry and YFP) or luminescent proteins (for example Renilla luciferase, Firefly luciferase), chromoproteins, enzymes that catalyse detectable reactions (for example β-Glucuronidase, β-Galactosidase), or selectable markers such as antibiotic resistance genes. Further examples of reporter genes are those listed in the Registry of Standard Biological Parts (http://parts.igem.org/Protein_coding_sequences/Reporters).

The terms “fragment” or “fragment of the coding region of a reporter gene” or similar as used herein refer to a string of amino acids or an amino acid sequence typically of reduced length relative to the or a reference polypeptide and comprising, over the common portion, an amino acid sequence identical to the reference polypeptide. Such a fragment according to the disclosure may be, where appropriate, included in a larger polypeptide of which it is a constituent. The fragments referred to herein may be between 5, 6, 7, 8, 9, 10, 11, 12, 13, 14, 15, 20, 25, 30, 35, 40, 41 amino acids in length and up to 41, 45, 50, 55, 60, 65, 70, 75, 80, 85, 90, 95, 100, 120, 140, 160, 180, 200, 250, 300 or more amino acids in length.

### Vectors Comprising Multiple Mirtrons

The inventors have demonstrated successful delivery of two independently functional mirtrons in a single vector, both from the 5′UTR of the same transgene for expressing rhodopsin.

There are several reasons why it might be desirable to have multiple functional mirtrons in the same vector or embedded in the same transgene. Firstly, the inventors have demonstrated that the effect on target gene knock down of multiple mirtrons expressed from the same vector are additive. The mirtron guide strands may recognise the same or different sequences within the target gene. Using multiple mirtrons can thereby result in an increased or more robust knock-down effect on a target gene.

Secondly, including multiple mirtrons that recognise different sequences in the same target gene can ensure that a vector can be used to treat disease caused by mutations in any part of the target gene expressed in a subject in need of treatment. Significant mutational heterogeneity exists for example in the rhodopsin gene, so targeted suppression alone as a strategy is unlikely to be universally successful. However, if more than one mirtron is included in a gene therapy vector, then the vector is likely to be at least partly effective in knocking down expression of the target gene even in subjects that happen to have a mutation within the target sequence of one of the mirtrons.

Thirdly, multiple mirtrons could be used to knock down expression of multiple target genes using the same vector. This is particularly likely to be useful in the treatment of complex diseases in which several different genes are involved.

Fourthly it can be useful to include multiple mirtrons in a single vector for experimental purposes, for example to compare the relative efficacy of the mirtrons.

Accordingly in some cases in accordance with the present invention, the plasmid or vector may comprise two or more copies of the same mirtron, or two or more mirtrons that complement the same target sequence, or two or more different mirtrons that complement the different target sequences in the same or two or more different target genes.

In an example, described herein is a triple mirtron polynucleotide, gene therapy vector comprising the polynucleotide, and gene therapy vector that contains M3 and M5 plus an additional copy of M3 (the 5′UTR of the triple mirtron vector comprising or consisting of M3 and M5 plus an additional copy of M3 is set forth in SEQ ID NO: 72, see Examples 27 and 28 below).

In some cases the two or more mirtrons may each be embedded in the 5′UTR of a transgene for co-expression with the mirtrons. One or more or each of the mirtrons may be flanked by a polynucleotide sequence that comprises ESEs and/or a fragment of a the coding region of a reporter gene as described above.

### Transgenes

The vectors of the present invention further comprise a gene for expression from the vector. This gene may be referred to herein as a “transgene”. The transgene may be for expression of a protein. In some cases the vector is a gene therapy vector. The transgene may be any gene the expression of which in a subject would provide therapeutic benefit to the subject, either alone or in combination with other therapeutic measures, such as co-expression of a mirtron.

In some cases the transgene is a variant of the gene that is targeted by the mirtron. A vector comprising both transgene and mirtron may be used to both knock down expression of a gene using the mirtron and to replace it with expression of the variant gene. This may be referred to as “block and replace” treatment or use of a “block and replace” vector. The variant gene is selected to be resistant or “hardened” to knock down by the mirtron. Typically the variant gene differs from the target gene in the polynucleotide target sequence that is recognised by the mirtron guide strand, typically by the substitution of one or more, in some cases 2, 3, 4, 5, 6, 7, 8, 9 or 10 or more nucleotides in the target sequence. For example, in the transgene one or more codons may be substituted compared with the codons in the target sequence recognised by the mirtron with alternative codons that encode the same amino acids. Ideally ‘rare’ codons are avoided. The transgene is not targeted for knock-down by the mirtron because the guide strand does not complement any sequence in the variant gene.

### Vectors

In some embodiments, the invention relates to plasmids, vectors, or gene therapy vectors. A gene therapy vector is any vector suitable for use in gene therapy, i.e. any vector suitable for the therapeutic delivery of nucleic acid polymers into target cells. The vector may be of any type, for example it may be a plasmid vector or a minicircle DNA. Typically, the vector is a viral vector. The viral vector may for example be derived from an adeno-associated virus (AAV), a retrovirus, a lentivirus, a herpes simplex virus, or an adenovirus.

### AAV Derived Vectors

The vector may comprise an AAV genome or a derivative thereof. An AAV genome is a polynucleotide sequence which encodes functions needed for production of an AAV viral particle. These functions include those operating in the replication and packaging cycle for AAV in a host cell, including encapsidation of the AAV genome into an AAV viral particle. Naturally occurring AAV viruses are replication-deficient and rely on the provision of helper functions in trans for completion of a replication and packaging cycle. Accordingly, the AAV genome of the vector of the invention is typically replication-deficient.

The AAV genome may be in single-stranded form, either positive or negative-sense, or in double-stranded form. The use of a double-stranded form allows bypass of the DNA replication step in the target cell and so can accelerate transgene expression.

Typically, the AAV genome of a naturally derived AAV comprises at least one inverted terminal repeat sequence (ITR). An ITR sequence acts in cis to provide a functional origin of replication, and allows for integration and excision of the vector from the genome of a cell. The AAV genome may also comprises packaging genes, such as rep and/or cap genes which encode packaging functions for an AAV viral particle. The rep gene encodes one or more of the proteins Rep78, Rep68, Rep52 and Rep40 or variants thereof. The cap gene encodes one or more capsid proteins such as VP1, VP2 and VP3 or variants thereof. These proteins make up the capsid of an AAV viral particle. Capsid variants are discussed below.

A promoter may be operably linked to each of the packaging genes. Specific examples of such promoters include the p5, p19 and p40 promoters (Laughlin et al., 1979, PNAS, 76:5567-5571). For example, the p5 and p19 promoters are generally used to express the rep gene, while the p40 promoter is generally used to express the cap gene.

The AAV genome may be from any naturally derived serotype or isolate or clade of AAV. Thus, the AAV genome may be the full genome of a naturally occurring AAV virus. As is known to the skilled person, AAV viruses occurring in nature may be classified according to various biological systems.

Commonly, AAV viruses are referred to in terms of their serotype. A serotype corresponds to a variant subspecies of AAV which, owing to its profile of expression of capsid surface antigens, has a distinctive reactivity that can be used to distinguish it from other variant subspecies. Typically, a virus having a particular AAV serotype does not efficiently cross-react with neutralising antibodies specific for any other AAV serotype. AAV serotypes include AAV1, AAV2, AAV3, AAV4, AAV5, AAV6, AAV7, AAV8, AAV9, AAV10 and AAV11, also recombinant serotypes, such as Rec2 and Rec3, identified from primate brain. The genome serotype of AAV for use in the invention may, for example, be AAV2. Reviews of AAV serotypes may be found in Choi et al. (Curr Gene Ther. 2005; 5(3); 299-310) and Wu et al (Molecular Therapy. 2006; 14(3), 316-327).

The sequences of AAV genomes or of elements of AAV genomes including ITR sequences, rep or cap genes for use in the invention may be derived from the following accession numbers for AAV whole genome sequences: Adeno-associated virus 1 NC_002077, AF063497; Adeno-associated virus 2 NC_001401; Adeno-associated virus 3 NC_001729; Adeno-associated virus 3B NC_001863; Adeno-associated virus 4 NC_001829; Adeno-associated virus 5 Y18065, AF085716; Adeno-associated virus 6 NC_001862; Avian AAV ATCC VR-865 AY186198, AY629583, NC_004828; Avian AAV strain DA-1 NC_006263, AY629583; Bovine AAV NC_005889, AY388617.

AAV viruses may also be referred to in terms of clades or clones. This refers to the phylogenetic relationship of naturally derived AAV viruses, and typically to a phylogenetic group of AAV viruses which can be traced back to a common ancestor, and includes all descendants thereof. Additionally, AAV viruses may be referred to in terms of a specific isolate, i.e. a genetic isolate of a specific AAV virus found in nature. The term genetic isolate describes a population of AAV viruses which has undergone limited genetic mixing with other naturally occurring AAV viruses, thereby defining a recognisably distinct population at a genetic level.

Examples of clades and isolates of AAV that may be used in the invention include:
Clade A: AAV1 NC_002077, AF063497, AAV6 NC_001862, Hu. 48 AY530611, Hu 43 AY530606, Hu 44 AY530607, Hu 46 AY530609
Clade B: Hu. 19 AY530584, Hu. 20 AY530586, Hu 23 AY530589, Hu22 AY530588, Hu24 AY530590, Hu21 AY530587, Hu27 AY530592, Hu28 AY530593, Hu 29 AY530594, Hu63 AY530624, Hu64 AY530625, Hu13 AY530578, Hu56 AY530618, Hu57 AY530619, Hu49 AY530612, Hu58 AY530620, Hu34 AY530598, Hu35 AY530599, AAV2 NC_001401, Hu45 AY530608, Hu47 AY530610, Hu51 AY530613, Hu52 AY530614, Hu T41 AY695378, Hu S17 AY695376, Hu T88 AY695375, Hu T71 AY695374, Hu T70 AY695373, Hu T40 AY695372, Hu T32 AY695371, Hu T17 AY695370, Hu LG15 AY695377,
Clade C: Hu9 AY530629, Hu10 AY530576, Hu11 AY530577, Hu53 AY530615, Hu55 AY530617, Hu54 AY530616, Hu7 AY530628, Hu18 AY530583, Hu15 AY530580, Hu16 AY530581, Hu25 AY530591, Hu60 AY530622, Ch5 AY243021, Hu3 AY530595, Hu1 AY530575, Hu4 AY530602 Hu2, AY530585, Hu61 AY530623
Clade D: Rh62 AY530573, Rh48 AY530561, Rh54 AY530567, Rh55 AY530568, Cy2 AY243020, AAV7 AF513851, Rh35 AY243000, Rh37 AY242998, Rh36 AY242999, Cy6 AY243016, Cy4 AY243018, Cy3 AY243019, Cy5 AY243017, Rh13 AY243013
Clade E: Rh38 AY530558, Hu66 AY530626, Hu42 AY530605, Hu67 AY530627, Hu40 AY530603, Hu41 AY530604, Hu37 AY530600, Rh40 AY530559, Rh2 AY243007, Bb1 AY243023, Bb2 AY243022, Rh10 AY243015, Hu17 AY530582, Hu6 AY530621, Rh25 AY530557, Pi2 AY530554, Pi1 AY530553, Pi3 AY530555, Rh57 AY530569, Rh50 AY530563, Rh49 AY530562, Hu39 AY530601, Rh58 AY530570, Rh61 AY530572, Rh52 AY530565, Rh53 AY530566, Rh51 AY530564, Rh64 AY530574, Rh43 AY530560, AAV8 AF513852, Rh8 AY242997, Rh1 AY530556
Clade F: Hu14 (AAV9) AY530579, Hu31 AY530596, Hu32 AY530597, Clonal Isolate AAV5 Y18065, AF085716, AAV 3 NC_001729, AAV 3B NC_001863, AAV4 NC_001829, Rh34 AY243001, Rh33 AY243002, Rh32 AY243003/

The skilled person can select an appropriate serotype, clade, clone or isolate of AAV for use in the present invention on the basis of their common general knowledge. It should be understood however that the invention also encompasses use of an AAV genome of other serotypes that may not yet have been identified or characterised.

The AAV genome used in the invention may be the full genome of a naturally occurring AAV virus. However, while such a vector may in principle be administered to patients, this will be done rarely in practice. The AAV genome may instead be derivatised for the purpose of administration to patients. Such derivatisation is standard in the art and the present invention encompasses the use of any known derivative of an AAV genome, and derivatives which could be generated by applying techniques known in the art. Derivatisation of the AAV genome and of the AAV capsid (discussed below) are reviewed in Coura and Nardi (Virology Journal, 2007, 4:99), and in Choi et al. and Wu et al., referenced above.

Derivatives of an AAV genome include any truncated or modified forms of an AAV genome which allow for expression of a mirtron from the vector in vivo in accordance with the present invention. Typically, it is possible to truncate the AAV genome significantly to include minimal viral sequence yet retain the above function. Reducing the size of the AAV genome in this way allows for increased flexibility in incorporating one or more transgenes and other sequence elements such as mirtrons or regulatory elements within the vector. It may also reduce the possibility of integration of the vector into the host cell genome, reduce the risk of recombination of the vector with wild-type virus, and avoid the triggering of a cellular immune response to viral gene proteins in the target cell.

Typically, a derivative will include at least one inverted terminal repeat sequence (ITR), or two ITRs or more. One or more of the ITRs may be derived from AAV genomes having different serotypes, or may be a chimeric or mutant ITR. An example mutant ITR is one having a deletion of a trs (terminal resolution site). This deletion allows for continued replication of the genome to generate a single-stranded genome which contains both coding and complementary sequences i.e. a self-complementary AAV genome. This allows for bypass of DNA replication in the target cell, and so enables accelerated transgene expression.

The one or more ITRs may flank a polynucleotide sequence encoding a mirtron and/or a transgene polypeptide at either end. The inclusion of one or more ITRs may aid concatamer formation of the vector of the invention in the nucleus of a host cell, for example following the conversion of single-stranded vector DNA into double-stranded DNA by the action of host cell DNA polymerases. The formation of such episomal concatamers protects the vector construct during the life of the host cell, thereby allowing for prolonged expression of the transgene in vivo. The ITR sequences may, for example, be those of AAV2 having, for example, the sequence of SEQ ID NOs 32 and 33 or variants thereof.

In some embodiments, ITR elements may be the only sequences retained from the native AAV genome in the derivative. Such a derivative will not include the rep and/or cap genes of the native genome or any other sequences of the native genome.

The following portions could therefore be removed in a derivative of the invention: One inverted terminal repeat (ITR) sequence, the replication (rep) and capsid (cap) genes. However, in some embodiments, derivatives may additionally include one or more rep and/or cap genes or other viral sequences of an AAV genome. Naturally occurring AAV virus integrates with a high frequency at a specific site on human chromosome 19, and shows a negligible frequency of random integration, such that retention of an integrative capacity in the vector may be tolerated in a therapeutic setting.

The derivative may be a chimeric, shuffled or capsid modified derivative. Chimeric, shuffled or capsid-modified derivatives will be typically selected to provide one or more desired functionalities for the viral vector. Thus, these derivatives may display increased efficiency of gene delivery, decreased immunogenicity (humoral or cellular), an altered tropism range and/or improved targeting of a particular cell type compared to an AAV viral vector comprising a naturally occurring AAV genome, such as that of AAV2, AAV5, AAV6, AAV8 or AAV9. Increased efficiency of gene delivery may be effected by improved receptor or co-receptor binding at the cell surface, improved internalisation, improved trafficking within the cell and into the nucleus, improved uncoating of the viral particle and improved conversion of a single-stranded genome to double-stranded form. Increased efficiency may also relate to an altered tropism range or targeting of a specific cell population, such that the vector dose is not diluted by administration to tissues where it is not needed.

The invention additionally encompasses the provision of sequences of an AAV genome in a different order and configuration to that of a native AAV genome. The invention also encompasses the replacement of one or more AAV sequences or genes with sequences from another virus or with chimeric genes composed of sequences from more than one virus. Such chimeric genes may be composed of sequences from two or more related viral proteins of different viral species.

### AAV Capsid Coat

A vector comprising an adeno-associated virus (AAV) genome or a derivative thereof may have a capsid coat. Such an encapsidated vector may be referred to as an AAV viral particle.

The AAV vectors or particles of the invention include transcapsidated forms wherein an AAV genome or derivative having an ITR of one serotype, for example AAV2, is packaged in the capsid of a different serotype, for example AAV8. The AAV vectors or particles of the invention also include mosaic forms wherein a mixture of unmodified capsid proteins from two or more different serotypes makes up the viral coat. The coat may also comprise modified capsid proteins or variants. The invention encompasses the provision of capsid protein sequences from different serotypes, clades, clones, or isolates of AAV within the same vector or AVV viral particle, i.e. pseudotyping.

The AAV capsid may determine the tissue specificity of infection (or tropism) of an AAV virus. Accordingly, the AAV capsid serotypes for use in the invention may be those which have natural tropism for or a high efficiency of infection of the target cells. For example, AAV8 capsid serotypes have a natural tropism for cells of the retina, whilst AAV2 and AAV9 have a natural tropism for neurons. Further, one or more of the capsid proteins may be a variant of a capsid protein, such as a chimeric, shuffled, or modified variant capsid protein.

Chimeric capsid proteins include those generated by recombination between two or more capsid coding sequences of naturally occurring AAV serotypes. This may be performed for example by a marker rescue approach in which non-infectious capsid sequences of one serotype are cotransfected with capsid sequences of a different serotype, and directed selection is used to select for capsid sequences having desired properties. The capsid sequences of the different serotypes can be altered by homologous recombination within the cell to produce novel chimeric capsid proteins.

Chimeric capsid proteins also include those generated by engineering of capsid protein sequences to transfer specific capsid protein domains, surface loops or specific amino acid residues between two or more capsid proteins, for example between two or more capsid proteins of different serotypes.

Shuffled or chimeric capsid proteins may also be generated by DNA shuffling or by error-prone PCR. Hybrid AAV capsid genes can be created by randomly fragmenting the sequences of related AAV genes e.g. those encoding capsid proteins of multiple different serotypes and then subsequently reassembling the fragments in a self-priming polymerase reaction, which may also cause crossovers in regions of sequence homology. A library of hybrid AAV genes created in this way by shuffling the capsid genes of several serotypes can be screened to identify viral clones having a desired functionality. Similarly, error prone PCR may be used to randomly mutate AAV capsid genes to create a diverse library of variants which may then be selected for a desired property.

The sequences of the capsid genes may also be genetically modified to introduce specific deletions, substitutions or insertions with respect to the native wild-type sequence. In particular, capsid genes may be modified by the insertion of a sequence of an unrelated protein or peptide within an open reading frame of a capsid coding sequence, or at the N- and/or C-terminus of a capsid coding sequence.

The unrelated protein or peptide may advantageously be one which acts as a ligand for a particular cell type. It may thereby confer improved binding to a target cell or improve targeting or the specificity of targeting of the vector to a particular target cell population. Alternatively the unrelated protein may be one which assists purification of the viral particle as part of the production process i.e. an epitope or affinity tag. The site of insertion will typically be selected so as not to interfere with other functions of the viral particle e.g. internalisation, trafficking of the viral particle. The skilled person can identify suitable sites for insertion based on their common general knowledge. Particular sites are disclosed in Choi et al., referenced above. The AAV vector or particle also includes chemically modified forms bearing ligands adsorbed to the capsid surface. For example, such ligands may include antibodies for targeting a particular cell surface receptor.

Relevant sections of the description relating the AAV derived vectors also apply in the case of vectors derived from other sources, such as those discussed further below.

### Retrovirus Derived Vectors

The vector may comprise a retrovirus genome or a derivative thereof. Derivatives of a retrovirus genome include any truncated or modified forms of a retrovirus genome which allow for expression of a mirtron from the vector in vivo in accordance with the present invention.

As with AAV derived vectors, a retrovirus derived vector will typically comprise a derivative of a retroviral genome comprising the minimal viral sequences required for packaging and subsequent integration into a host. For retrovirus derived vectors, one or more long terminal repeats (LTRs) are the minimum element required for replication and packaging of the vectors and subsequent integration into the target cell to provide permanent transgene expression. However, other elements may also be present. For example, a human immuno deficiency virus (HIV) derived vector will typically comprises the HIV 5′ LTR, which is necessary for integration into the host cell genome, the Psi signal, which is necessary for packaging of viral RNA into virions, a promoter for the transgene, and the 3′ LTR. Other suitable retroviral vectors may for example be derived from murine leukemia virus (MuLV), gibbon ape leukemia virus (GaLV), Simian Immuno deficiency virus (SIV), and combinations thereof.

The tropism of a retrovirus derived vector is determined by the viral envelope proteins. Targeting of the appropriate cells may be enhanced by incorporating ligands for the target cells into the viral envelope.

### Adenovirus Derived Vector

The vector may comprise an adenovirus genome or a derivative thereof. Derivatives of an adenovirus genome include any truncated or modified forms of an adenovirus genome which allow for expression of a mirtron from the vector in vivo in accordance with the present invention.

A large number of human adenoviral serotypes have been identified and they are categorized into six subgenera (A through F) based on nucleic acid comparisons, fibre protein characteristics, and biological properties. For example, group A includes serotypes 12 and 31, group B includes serotypes 3 and 7, group C includes serotypes 2 and 5, group D includes serotypes 8 and 30, group E includes serotype 4, and group F includes serotypes 40 and 41.

The core of an adenovirus virion contains the linear double-stranded DNA genome and associated proteins V, VII, X (mu), IVa2, and terminal protein (TP). The genome organization of different adenoviruses is conserved and has been proposed to have a timing function, wherein the ends of the genome are transcribed first (the immediate early genes E1 and E4 are located at opposite ends of the linear genome). Early transcription of E1 and E4 leads to the opening of the central region of the genome, allowing transcription of the central region.

Adenoviral genomes typically comprise eight RNA polymerase II transcriptional units: five early units, E1A, E1B, E2A-E2B, E3, and E4; two delayed early units, IX and IVa2; and the Major Late transcriptional unit. The Major Late transcriptional unit is further subdivided into L1-L5 regions based upon the use of alternative splicing sites. The transcriptional units often express proteins of similar function. For example, the E1A unit codes for two proteins responsible for activation of transcription and induction of S-phase upon cellular infection; the E1B transcription unit encodes two proteins that inhibit cellular apoptosis; the E3 transcriptional unit is involved in evasion of the immune response; and the Major Late transcriptional unit encodes structural proteins necessary for assembly of the capsid.

Heterologous mirtron and/or transgene sequences may be inserted into adenoviral genomes, for example in the early transcriptional units and in the coding regions of various structural proteins, such as hexon, penton, and fiber. Deletions may have been made in the adenoviral genome (e.g., in the E1 regions) to create replication-defective adenoviral vectors, which have generally been considered safer for administration to human subjects.

In the present invention, the adenovirus may be any adenovirus or derivative suitable for delivery of the transgene to target cells. The adenovirus may be any serotype but is typically Ad5 or Ad2. An adenovirus derived vector of the invention may comprise all or part of the genome of any adenoviral serotype, as well as combinations thereof (i.e., hybrid genomes).

The adenoviral vector used in the invention may be either replication incompetent or replication competent. Such vectors are well known. For example, in a replication incompetent vector the E1 region may be deleted and replaced with an expression cassette with an exogenous promoter driving expression of the heterologous transgene. Usually, the E3 region is also deleted. Deletion of E3 allows for larger inserts into the E1 region. Such vectors may be propagated in appropriate cell lines such as HEK 293 cells which retain and express the E1A and E1B proteins. Other vectors also lack the E4 region, and some vectors further lack the E2 region. E2 and E4 vectors must be grown on cell lines that complement the E1, E4 and E2 deletions.

Vectors may also be helper dependent vectors, which lack most or all of the adenoviral genes but retain cis-acting sequences such as the inverted terminal repeats as well as packaging sequences that are required for the genome to be packaged and replicated. These vectors are propagated in the presence of a helper adenovirus, which must be eliminated from the vector stocks. Once again, such systems are well known in the art.

The capsid is composed of seven structural proteins: II (hexon), III (penton), IIIa, IV (fiber), VI, VII, and IX. The capsid comprises 252 capsomeres, of which 240 are hexon capsomeres and 12 are penton capsomeres. Hexon capsomeres, which are trimers of the hexon protein, make up about 75% of the protein of the capsid. Penton capsomeres, which are pentamers of the penton protein, are situated at each of the 12 vertices of the virion. Each penton capsomer is bound to six adjacent hexon capsomeres and a fiber. The fiber, which is usually a trimer of the fiber protein, projects from the penton capsomer. The hexon protein and, to a lesser extent, the fiber protein comprise the main antigenic determinants of an adenovirus and also determine serotype specificity.

An adenovirus derived vector is particularly suitable for use in the invention when a transient expression of a mirtron and/or transgene is preferred.

### Herpes Simplex Virus Derived Vectors

The vector may comprise an herpes simplex virus (HSV) genome or a derivative thereof. Derivatives of an HSV genome include any truncated or modified forms of a HSV genome which allow for expression of a mirtron from the vector in vivo in accordance with the present invention.

Herpes simplex virus (HSV) naturally establishes a life-long latent infection of human peripheral sensory neurons. Recombinant HSV vectors are genetically modified to be incapable of replication, but establish a latent-like state in neurons in vitro and in vivo.

### Promoters and Other Regulatory Elements

In the plasmid or vector, the nucleic acid encoding the mirtron and transgene is typically operably linked to a promoter. Any suitable promoter may be used as are well known in the art. The promoter may be constitutive i.e. operational in any host cell background. The promoter may for example be the ubiquitous CAG promoter. Alternatively, the promoter may be a cell-specific promoter, which drives expression a particular target cell type. For example, the human rhodopsin promoter, drives expression only in rod photoreceptor cells.

One or more other regulatory elements, such as enhancers, postregulatory elements and polyadenylation sites may also be present in addition to the promoter. A regulatory sequence that is operably linked to the transgene and/or mirtron is any sequences that facilitates or controls expression of the transgene, for example by promoting or otherwise regulating transcription, processing, nuclear export of mRNA or stability. The term “operably linked” refers to a juxtaposition wherein the components described are in a relationship permitting them to function in their intended manner. A control sequence (e.g. a promoter) “operably linked” to a mirtron or transgene is ligated in such a way that expression of the mirtron or transgene is achieved under conditions compatible with the control sequences.

### Preparation of Vector

A vector of the invention may be prepared by standard means known in the art for provision of vectors for gene therapy. Thus, well established public domain transfection, packaging and purification methods can be used to prepare a suitable vector.

Viral vectors used in gene therapy are typically generated by producing a cell line that packages a nucleic acid vector into a viral particle. The vectors typically contain the minimal viral sequences required for packaging and subsequent integration into a host, as exemplified above, other viral sequences being deleted, leaving capacity for an expression cassette for the polynucleotide(s) to be expressed, such as a mirtron or transgene. The missing viral functions are typically supplied in trans by the packaging cell line.

Packaging cells are typically used to form virus particles that are capable of infecting a host cell. The packaging cells may be any suitable cell type known in the art. The packaging cells are typically human or human derived cells. Suitable cells include Human Embryonic Kidney (HEK) 293 cells, or HEK 293 derived cell clones (for example to package adenovirus derived vectors), HeLa cells (for example to package HIV or other lentivirus derived vectors) and ψ2 cells or PA317 cells (for example to package retrovirus derived vectors).

AAV derived vectors of the invention may comprise the full genome of a naturally occurring AAV virus in addition to the elements for gene therapy such as a mirtron or transgene. However, commonly a derivatised genome will be used, for instance a derivative which has at least one inverted terminal repeat sequence (ITR), but which may lack any AAV genes such as rep or cap.

In such embodiments, in order to provide for assembly of the derivatised genome into an AAV viral particle, additional genetic constructs providing AAV and/or helper virus functions will be provided in a host cell in combination with the derivatised genome. These additional constructs will typically contain genes encoding structural AAV capsid proteins i.e. cap, VP1, VP2, VP3, and genes encoding other functions required for the AAV life cycle, such as rep. The selection of structural capsid proteins provided on the additional construct will determine the serotype of the packaged viral vector.

AAV viruses are replication incompetent and so helper virus functions, preferably adenovirus helper functions will typically also be provided on one or more additional constructs to allow for AAV replication.

The additional constructs may be provided as plasmids or other episomal elements in the host cell, or alternatively one or more constructs may be integrated into the genome of the host cell.

### Methods of Therapy and Medical Uses

The invention concerns methods of treatment of a retinal disease and vectors for use in such methods. The method may be a method of gene therapy. The term “gene therapy” means the therapeutic delivery of nucleic acid polymers into a patient's cells. In some cases of gene therapy, copies of one or more genes that encode a protein that is therapeutic to a subject are introduced to cells of the subject. Such a gene for introduction to the cells may be referred to herein as a transgene. In some cases the gene therapy introduces to the cells one or more genes that are normally expressed in a healthy individual but that are missing or defective in the subject. In other cases of gene therapy a nucleic acid polymer may be introduced to knock down expression of a gene in the subject, that is to reduce or inhibit expression of a gene product. According to the present invention the gene therapy comprises administration of a vector that comprises a mirtron for knocking down expression of a gene in a subject and a transgene for expressing a gene in the subject. The gene that is targeted for knock down by the mirtron may be referred to herein as an endogenous gene or target gene. The transgene may in some cases be referred to as an exogenous gene.

The disease that is treated may be any disease, condition or disorder that is caused by or exacerbated by the expression or over-expression of a gene in a subject. In some cases the disease may be caused by a mutation in the gene compared to a wild-type healthy gene. The disease may be dominant genetic (or autosomal dominant) in which a mutation in one of the two copies of the gene in a subject can be sufficient for the subject to be affected by the disease or for the disease to be exacerbated. In other cases the disease may be recessive genetic (or autosomal recessive), in which a subject must have mutations in both copies of the gene to be affected or for the disease to be exacerbated. In some cases the disease is caused by or primarily caused or triggered by mutation in a single gene. In other cases the present invention may be used to treat complex genetic diseases, in which multiple genes may be involved, possibly in combination with lifestyle or environmental contributory factors.

The subject may be a human or a non-human animal. Non-human animals include, but are not limited to, rodents (including mice and rats), and other common laboratory, domestic and agricultural animals, including rabbits, dogs, cats, horses, cows, sheep, goats, pigs, chickens, amphibians, reptiles etc.

### Pharmaceutical Compositions and Modes of Administration

The one or more vectors of the invention may be formulated into pharmaceutical compositions. These compositions may comprise, in addition to the vector(s), a pharmaceutically acceptable excipient, carrier, buffer, stabiliser or other materials well known to those skilled in the art. Such materials should be non-toxic and should not interfere with the efficacy of the active ingredient. Examples of suitable compositions and methods of administration are provided in Esseku and Adeyeye (2011) and Van den Mooter G. (2006). The precise nature of the carrier or other material may be determined by the skilled person according to the route of administration. Examples of techniques and protocols can be found in Remington's Pharmaceutical Sciences, 20th Edition, 2000, pub. Lippincott, Williams & Wilkins.

The vectors of the invention may be administered by any suitable route and means that allows for transduction of the target cells. In some embodiments the vectors may be administered systemically, for example by intravenous administration.

Dosages and dosage regimes can be determined within the normal skill of the medical practitioner responsible for administration of the composition. The dose of a vector of the invention may be determined according to various parameters, especially according to the age, weight and condition of the patient to be treated; the route of administration; and the required regimen. A physician will be able to determine the required route of administration and dosage for any particular patient.

Administration is typically in a “prophylactically effective amount” or a “therapeutically effective amount” (as the case may be, although prophylaxis may be considered therapy), this being sufficient to show benefit to the individual. For example, a therapeutically effective amount of a vector of the invention or an effective method of treatment in accordance with invention may result in reduced expression of the target gene of the mirtron and in some cases also expression of the transgene in target cells of the retina. The treatment is sufficient to result in a clinical response or to show clinical benefit to the individual, for example to cure the disease, prevent or delay onset or progression of the disease or condition or one or more symptoms, to ameliorate or alleviate one or more symptom, to induce or prolong remission, or to delay relapse or recurrence.

A typical single dose of the one or more vectors of the invention may between 10⁹ and 10¹⁵, or 10¹⁰ and 10¹⁴, or 10¹¹ and 10¹² viral genomes (vg), or any range thereof. A dose at the lower end of the range will typically be used for administration direct to the site of the target cells, whilst a dose at the higher end of the range will typically be needed for systemic administration. A single AAV capsid that contains a single stranded DNA molecule is a single viral genome (vg). Vg can be quantified by any suitable method as well known in the art, for example real-time PCR. The one or more vectors are preferably administered only once, resulting, depending on the vector used, in permanent or transient knock down of the target gene, but repeat administrations, for example in future years and/or with different serotypes may be considered.

A composition of the invention may be administered alone or in combination with other therapeutic compositions or treatments.

### EXAMPLES

### Example 1—Design of Transgene Cassettes for Expressing Rhodopsin In Vivo

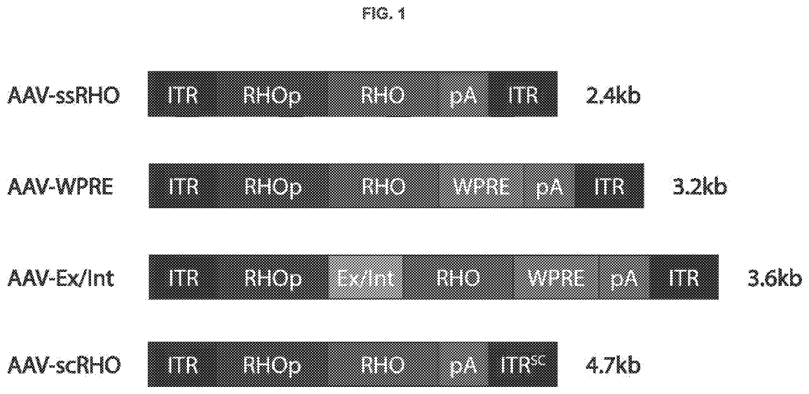

Four human rhodopsin expressing AAV2/8^{Y733F} vectors were designed and manufactured (FIG. 1). All four contained the human rhodopsin cDNA sequence (RHO; SEQ ID NO: 1) downstream of the human rhodopsin promoter (RHOp; SEQ ID NO: 34):
1. AAV-ssRHO (AAV2/8^{Y733F}.RHOp.RHO): a single-stranded AAV genome containing the human rhodopsin CDS under the control of the human rhodopsin promoter.
2. AAV-WPRE (AAV2/8^{Y733F}.RHOp.RHO.WPRE): as for AAV-ssRHO but with the addition of a 3′ Woodchuck Hepatitis Post-transcriptional Regulatory Element (WPRE) sequence (SEQ ID NO: 29). This sequence has been shown to improve transgene expression following retinal gene therapy in mice and humans (Patricio et al. (2017), Mol. Ther.—Nucleic Acids 6:198-208).
3. AAV-Ex/Int (AAV2/8^{Y733F}.RHOp.Ex/Int.RHO.WPRE): as for AAV-WPRE but with the addition of the first exon and intron of the chicken beta-actin gene together with the splice acceptor from the rabbit beta-globin gene located between the RHOp sequence and Kozak consensus.
4. AAV-scRHO (scAAV2/8^{Y733F}.RHOp.RHO): a self-complementary (sc) version of AAV-ssRHO. Self-complementary vectors lead to increased vector efficiency as they bypass intracellular second-strand synthesis, a step that is thought to be rate-limiting in the process of AAV transduction. One major drawback of sc vectors is that they have a packaging capacity that is effectively half that of their single-stranded (ss) counterparts. This vector is very close to capacity and so self-complementarity has to be an alternative to the additional elements included in vectors 2 and 3 above.

### Example 2—Ex Vivo Validation of Rhodopsin Expression Cassettes

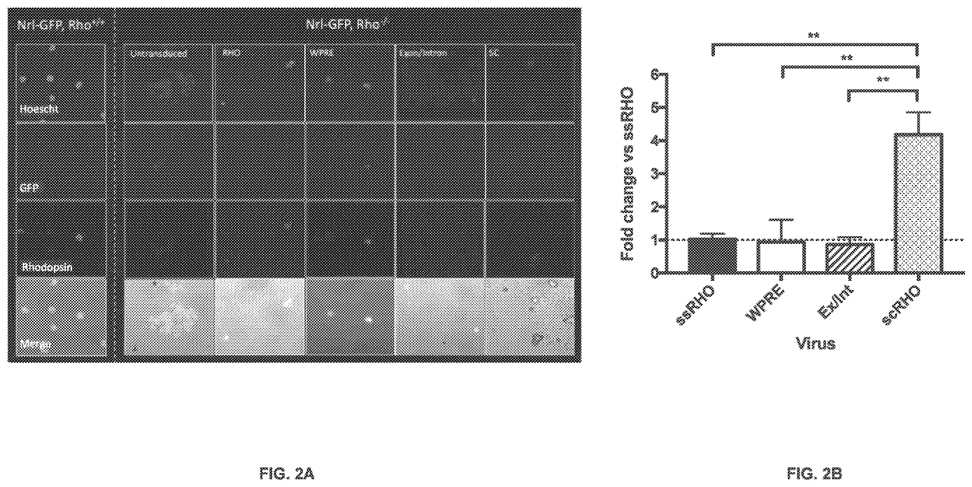

The four viruses were validated ex vivo using dissociated retinal cells from post-natal day (PND) 3 Nrl.GFP/Rho^{-/-} mice. These animals carry a homozygous null mutation in rhodopsin and express GFP under the neural retina leucine zipper (Nrl) promoter. Rod cells are thus fluorescence labelled green and express no native rhodopsin. Rescue of rhodopsin expression was noted following transduction with all four viruses and immunofluorescence staining for RHO protein (FIG. 2A). RNA was also extracted from dissociated transduced PND3 retinal cells and converted to cDNA. Quantitative PCR (qPCR) analysis revealed a fourfold increase in the number of rhodopsin transcripts for AAV-scRHO transduced cells compared with those transduced with the other three viruses (FIG. 2B).

### Example 3—In Vivo Validation of Rhodopsin Expression Cassettes

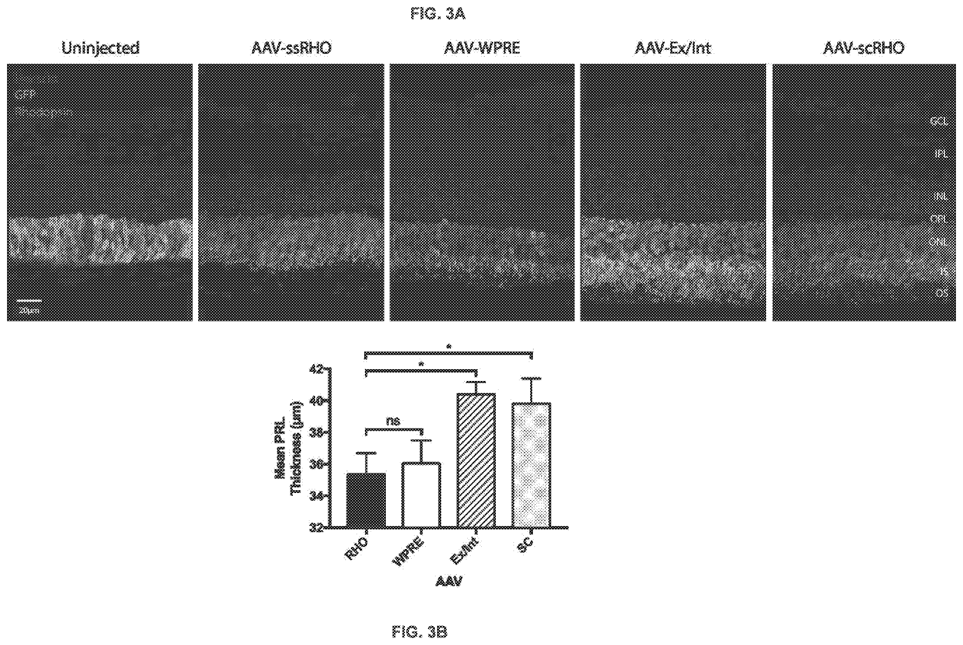

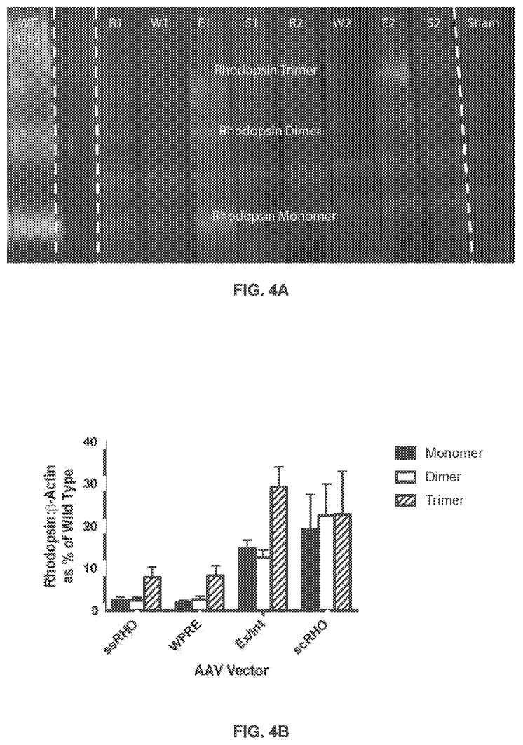

An in vivo comparison of the four vectors was subsequently undertaken. Subretinal injections of 2×10⁹ genome copies (gc) of each virus in 1.5 μl were performed in Nrl.GFP/Rho^{-/-} mice. Four weeks later, live mice were subject to spectral domain optical coherence tomography (SD-OCT) from which mean photoreceptor layer (PRL) thickness was calculated. Mice were then sacrificed and eyes extracted. Some were fixed, embedded and sectioned for immunohistochemistry, whilst others were processed for western blot analysis of rhodopsin protein levels. FIG. 3A shows retinal cryosections from such mice stained for rhodopsin. All four viruses were capable of driving human rhodopsin protein expression in the murine retina. Transduced rods elaborated outer segments packed with rhodopsin that were absent in untransduced eyes. Rhodopsin expression was confined to this layer mimicking the wild type state and confirming promoter specificity. Rhodopsin expression appeared greatest in AAV-Ex/Int and AAV-scRHO injected eyes. Analysis of SD-OCT images from injected animals confirmed that these two vectors resulted in the greatest mean photoreceptor layer (PRL) thickness (p<0.05 for each versus AAV-RHO and AAV-WPRE, one-way ANOVA Holm-Sidak multiple comparison test; FIG. 3B). Similarly, these two vectors resulted in the greatest level of rhodopsin protein expression as measured by Western blot band densitometry (FIG. 4).

Taken together, the above data suggest that the addition of WPRE and the chicken beta-actin/rabbit beta-globin exon-intron-exon sequence can allow similar levels of rod-specific transgene expression to be achieved as that associated with a self-complementary vector. The advantage of the optimized single-stranded construct is that additional packaging capacity remains to allow the additional inclusion of miRNA suppression elements (such as mirtrons).

### Example 4—Overexpression of Rhodopsin Alone does not Reduce Retinal Degeneration

Next, it was important to establish whether overexpression of rhodopsin alone was sufficient to rescue the degenerative phenotype in a mouse model of rhodopsin dominant retinitis pigmentosa. The P23H rhodopsin knock-in mouse (Jackson laboratories) was used for this and for subsequent vector testing as this represents the most biologically relevant mouse model available. Homozygous Rho^{P23H/P23H} mice were mated with homozygous Nrl.GFP mice to create offspring heterozygous for both alleles. These mice (which have rods labelled with GFP that degenerate over time) were used for all further testing and are henceforth simply referred to as ‘P23H’ mice.

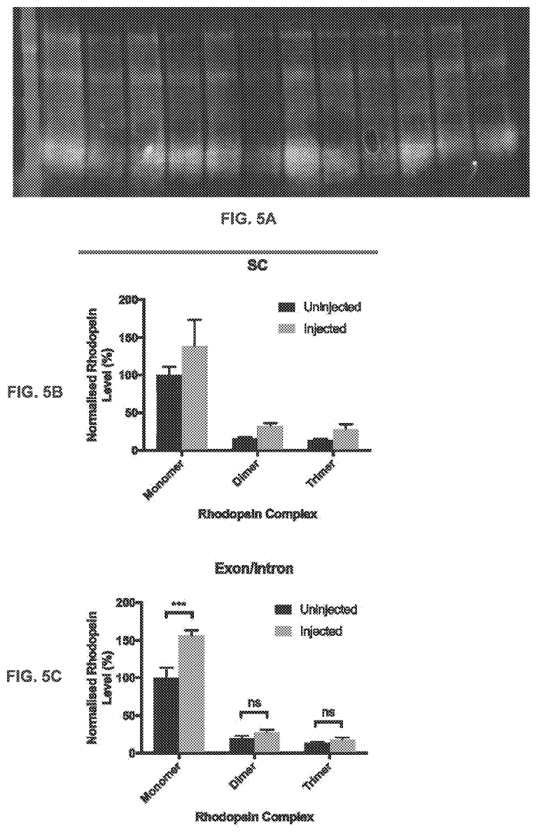

To test whether rhodopsin overexpression was achievable, P23H mice were injected with the two most successful rhodopsin supplementation vectors developed above, AAV-scRHO and AAV-Ex/Int. Subretinal injections of 2×10⁹ genome copies in 1.5 μl were performed in the right eyes of 3-week-old P23H mice (n=6 for each vector). Four weeks later, retinas were harvested and rhodopsin expression compared between injected right and uninjected left eyes by western blot band densitometry. This showed overexpression of rhodopsin of the order of 50% for both vectors (FIG. 5).

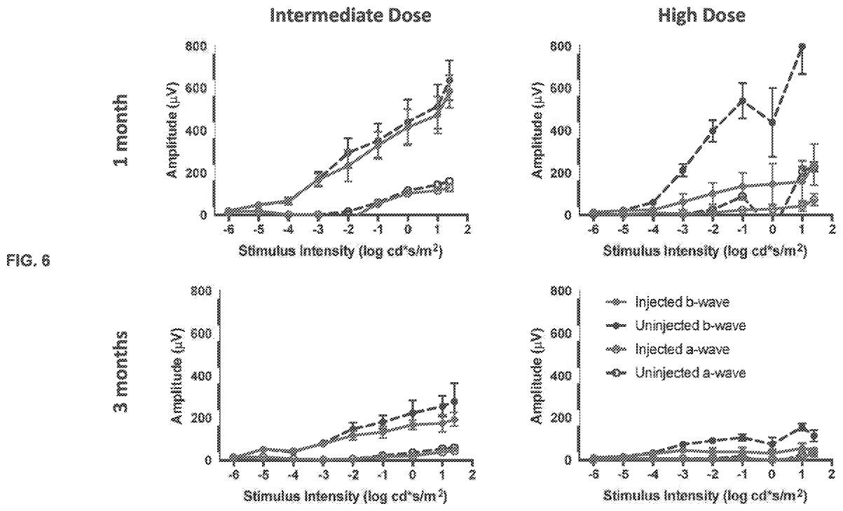

Next, pilot studies were performed to establish whether rhodopsin overexpression alone was sufficient to slow retinal degeneration in P23H mice. New cohorts of P23H mice were thus injected unilaterally with either AAV-scRHO at doses of 2×10⁸ gc (‘low dose’) or 2×10⁹ (‘intermediate dose’) or AAV-Ex/Int at doses of 2×10⁹ (‘intermediate dose’) or 2×10¹⁰ gc (‘high dose’). Electroretinography performed at one and three months post-injection showed that these treatments had no effect on the rate of retinal degeneration (FIG. 6).

### Example 5—Design of Mirtrons to Knock-Down Rhodopsin Expression

Having optimized the ‘replace’ arm of the ‘block and replace’ paradigm in vivo, laboratory studies were undertaken to design and manufacture a construct capable of knocking-down rhodopsin. Seven mirtrons were designed designated M1-M7, SEQ ID NO: 10 to 16). Each mirtron was engineered to target the human and/or mouse rhodopsin genes.

Double stranded mirtrons were engineered by annealing complementary oligonucleotides. Forward oligonucleotides had the general structure 5′-P-CGAAG-(shortened mirtron sequence) where P represents a phosphate group and the shortened mirtron sequence represents that of the 76 bp mirtron design minus the 3′ CGAAG motif (SEQ ID NOs: 41 to 47). Reverse oligonucleotides had the general structure 5′-P-(shortened reverse mirtron sequence)-CTT, where the shortened reverse mirtron sequence represents the reverse complement of the 76 bp mirtron sequence minus the 5′ CTT motif SEQ ID NOs: 48-54). Oligonucleotides were manufactured by Sigma-Aldrich, UK and reconstituted to a concentration of 10 μM upon receipt. 10 μl forward and 10 μl reverse solution was thoroughly mixed and heated to a temperature of 95° C. using a PCR thermocycler (Multigene, Labnet International Inc.). Samples were then slowly cooled in a stepwise manner to room temperature over a period of 30 minutes to facilitate efficient annealing.

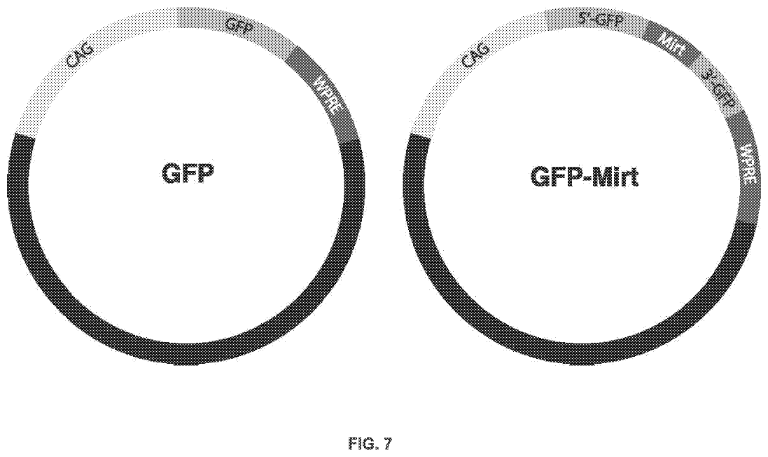

The resulting double stranded mirtron sequences (which by design already had BstB1 sticky ends) were each individually ligated into BstB1/shrimp alkaline phosphatase (both New England Biolabs, UK) treated CAG.eGFP.WPRE plasmid (FIG. 7). Correct orientation of the mirtron between nucleotides 346 and 347 of the eGFP coding region (SEQ ID NO: 35) within the construct was confirmed by commercial Sanger sequencing (Source Biosciences, UK).

### Example 6—Quantification of Correct Mirtron Splicing

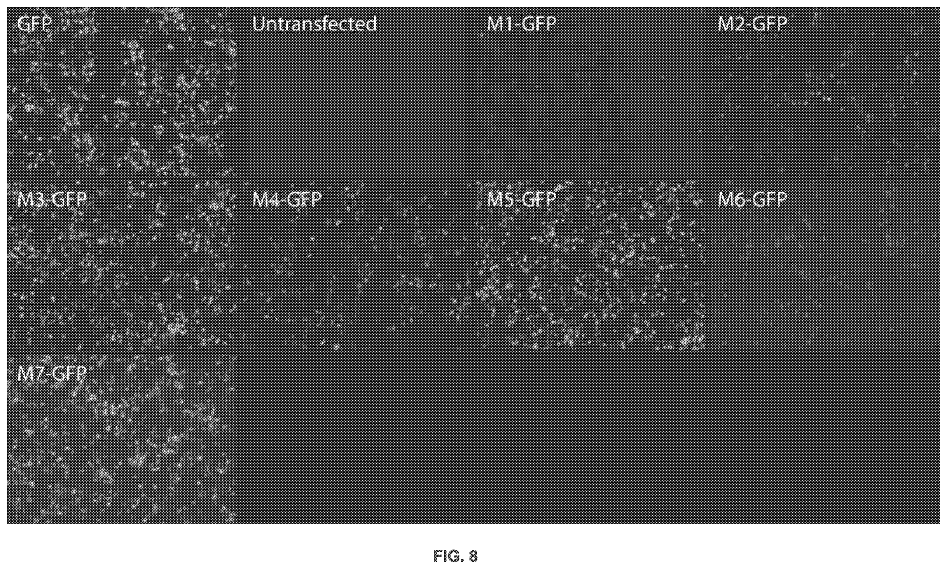

Each of the plasmids described in Example 5 were individually transfected into HEK293 cells. In each case, green fluorescence indicated successful and precise splicing out of the mirtron, as failure to splice would result in a frame shift of the GFP CDS downstream of the mirtron site. In this way, the degree of correct splicing for each mirtron design could be readily established in vitro and was measured using a quantitative fluorescence assay (fluorescence plate reading of GFP-mirtron transfected cell lysate normalised to that of GFP transfected cell lysate) (FIGS. 8 & 9A).

### Example 7—Determination of Mirtron Splice Variants

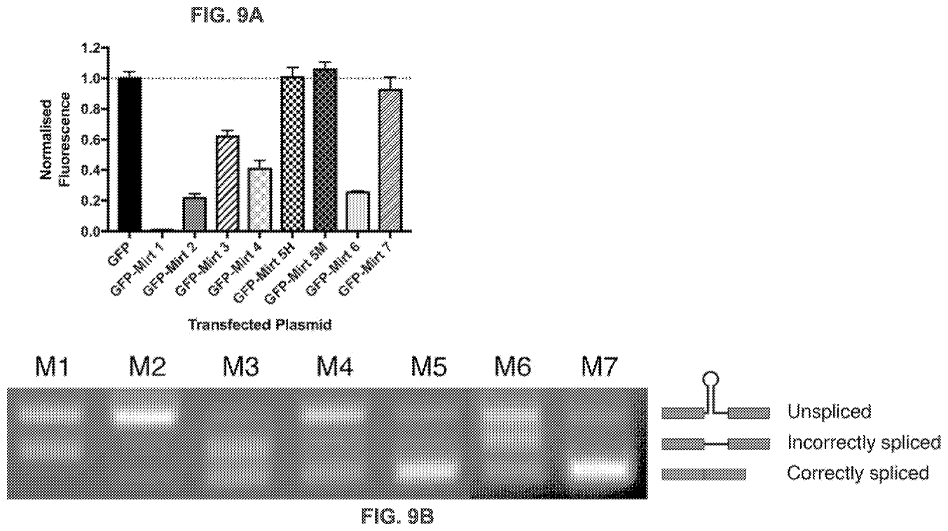

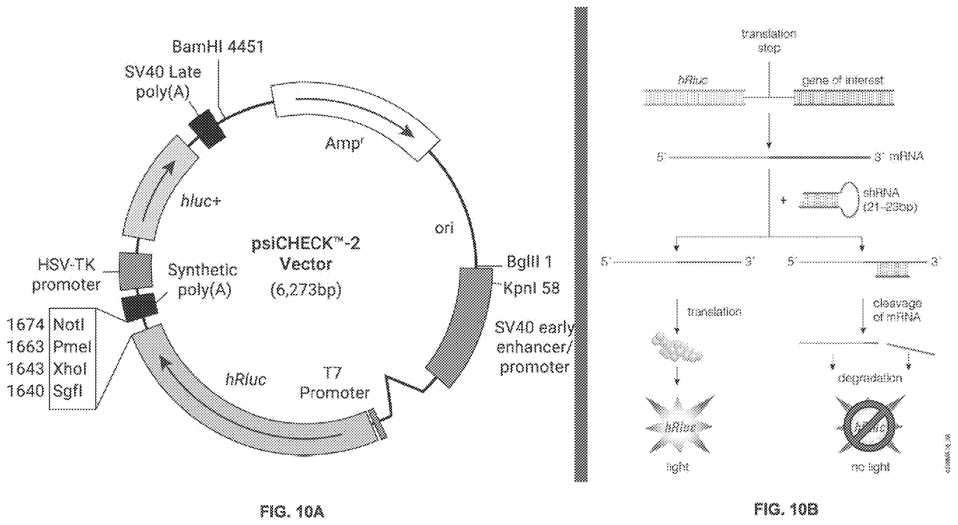

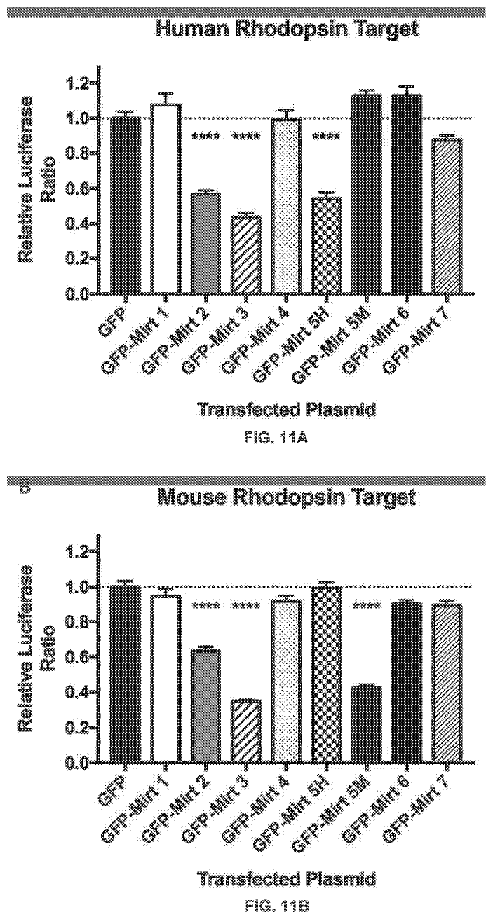

Splice variants for GFP-mirtron constructs were also determined. RNA from GFP-mirtron transfected cells was extracted and reverse transcribed into cDNA. A set of mirtron-spanning primers directed against the flanking GFP sequence was used to amplify the spliced region. The resulting PCR products were run through a 2% agarose gel by electrophoresis. Each resulting band corresponded to a different splice product. The proportion of correctly spliced product as determined by band densitometry corresponded to that predicted by the fluorescence splice assay outlined above (FIG. 9B).

### Example 8—In Vitro Determination of Mirtron-Mediated Knock Down: Target Sequence in 3′UTR

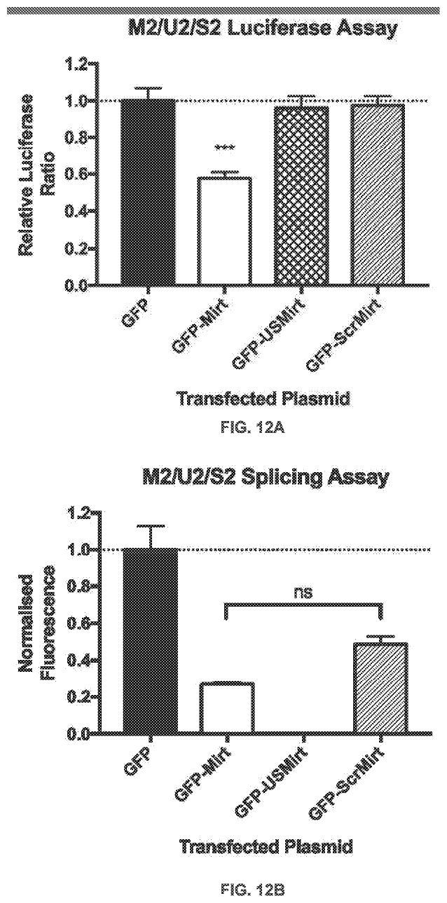

Rhodopsin knockdown efficiency for all seven mirtrons was determined in vitro using the luciferase assay in conjunction with the vector system (both Promega) according to manufacturer's instructions. Briefly, the plasmid contains two luciferase genes (Renilla and Firefly) which are independently under the control of ubiquitous promoters. Each luciferase sequentially catalyses a reaction that produces light which may be quantified with a luminometer (assay) (FIG. 10). The Renilla luciferase has a multiple cloning site located between its stop codon and polyA sequence into which rhodopsin coding sequences were cloned. HEK293 cells were co-transfected with this vector and each GFP-mirtron plasmid. An effective mirtron would be expected to cleave its target sequence thus deadenylating the Renilla luciferase transcript and reducing the Renilla luminescent signal. Mirtrons should not however affect the Firefly signal which acts as an internal transfection control in the assay. In this way, the relative luciferase ratio (Renilla:Firefly), which is normalised to a no-mirtron GFP control reading, quantifies mirtron mediated knockdown efficiency. All seven mirtron designs were tested against vectors for both human and mouse rhodopsin. The guide strands of mirtrons M1-4 and M6 were perfect antisense matches for both mouse and human rhodopsin mRNA. Separate human and mouse versions of mirtron 5 (designated ‘M5H’ and ‘M5M’, SEQ ID NO: 55; forward oligo: SEQ ID NO: 56; reverse oligo: SEQ ID NO: 57) were cloned which differed by 2 of the 21 guide strand nucleotides according to corresponding differences in the target nucleotide sequence of the two species. Mirtron 7 was a perfect antisense match with the human rhodopsin sequence but had a single nucleotide mismatch with the corresponding target in mouse. Results are shown in FIG. 11. Mirtrons 2, 3 and 5 induced a significant knock-down of rhodopsin of 40-70%. Note that M5H and M5M were only effective when targeting their corresponding species of rhodopsin.

### Example 9—Both Splicing and a Specific Antisense Sequence are Required for Mirtron-Induced RNA Knock-Down

The requirement of correct splicing and a specific antisense sequence for mirtron-mediated rhodopsin knockdown was investigated by cloning two alternative versions of Mirtron 2 (M2). The first was a so-called ‘unspliceable’ version (M2U, SEQ ID NO: 58; forward oligo: SEQ ID NO: 59; reverse oligo: SEQ ID NO: 60). M2 and M2U were identical in all but the first nucleotide (the ‘G’ at position 1 of the canonical splice donor site) which was substituted for an adenine nucleotide in M2U. The second was a ‘scrambled’ version (M2S, SEQ ID NO: 61; forward oligo: SEQ ID NO: 62; reverse oligo: SEQ ID NO: 63) where the nucleotides of the guide strand not predicted to be involved in splicing (positions 7-21) were randomly re-ordered. FIGS. 12A-B shows that M2U did not splice out of GFP (indicated by lack of green fluorescence) and was incapable of mediating rhodopsin knock-down. Although M2S spliced out to a similar degree to M2, it too did not induce rhodopsin knockdown. Thus, both splicing and a specific antisense sequence are required for mirtron-induced RNA knock-down.

### Example 10—Effect of Target Sequence Context on Mirtron Potency

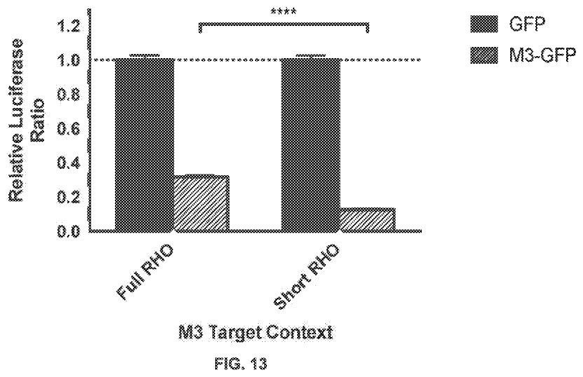

To investigate the effect of target sequence context on mirtron potency, a ‘short’ version of the human rhodopsin target comprising the 21 bp mirtron 3 (M3) target sequence with 25 bp of upstream and downstream DNA was cloned into the multiple cloning site of the vector. M3 efficacy against this construct was compared with that measured against the full-length sequence (FIG. 13). Interestingly, M3 was significantly more effective against the RHO target with shorter flanking sequence. Context of the target site can thus have a significant impact on the potency of mirtrons. This is likely attributable to local differences in mRNA secondary structure which may variously allow or restrict access of the RISC to the transcript target site, and may explain the differences observed between human and mouse targets for mirtrons with identical target sequences (see FIG. 11).

### Example 11—In Vitro Determination of Mirtron-Mediated Knock Down: Target Sequence in Coding Region

The luciferase assay quantifies efficacy of RNAi effectors which target the gene of interest cloned into the 3′ UTR of Renilla luciferase. It does not necessarily follow that mRNA cleavage will occur to the same extent in vivo where the target site is located within a CDS that is being actively translated. Indeed, it is thought that native miRNAs that target coding sequences may exert their effect more through translational inhibition rather than mRNA cleavage, and such miRNAs seem to have a smaller influence on target protein levels.

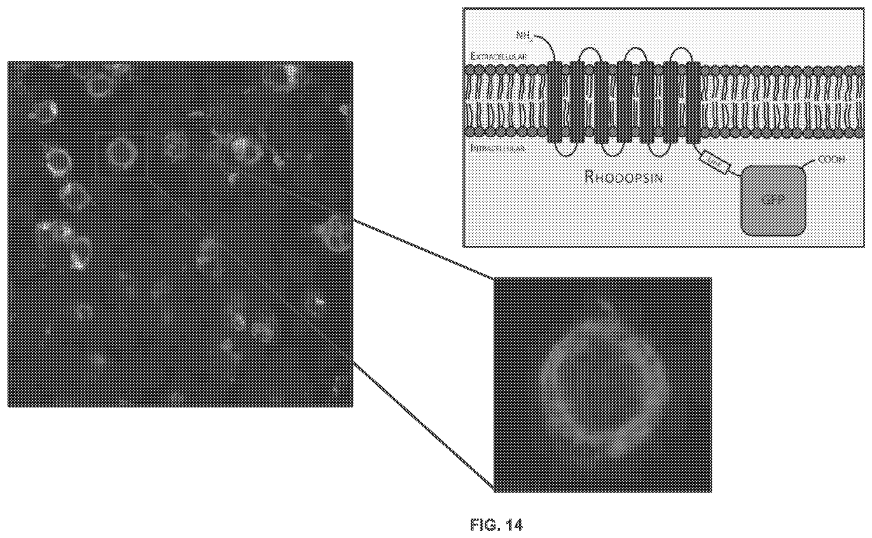

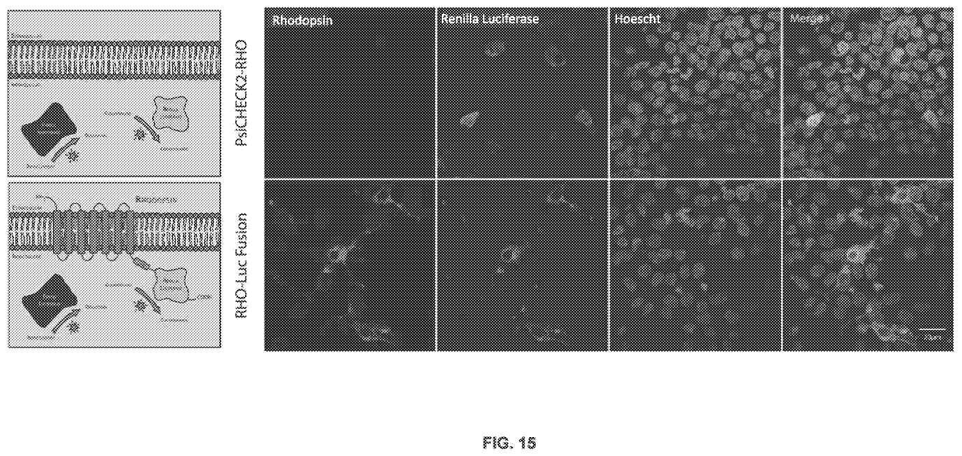

To investigate the efficacy of mirtrons directed against a translated sequence, the human rhodopsin gene was cloned into the vector so as to create a RHO-Renilla luciferase fusion protein with the luciferase being tagged to the cytosolic C-terminus of rhodopsin via an APVAT link peptide (SEQ ID NO:71). The validity of this approach was first established by cloning a rhodopsin-link peptide-GFP sequence into the CAG/WPRE plasmid backbone. This was used to transfect HEK293 cells that were subsequently immunostained using the 4D2 anti-rhodopsin antibody which targets the extracellular N-terminus of the protein. Confocal fluorescence microscopy revealed the RHO-GFP fusion protein to be localised to the plasma membrane, the protein being visible as a double ring of fluorescence (FIG. 14). HEK293 cells were then transfected with the RHO-Renilla luciferase fusion plasmid and stained for rhodopsin and luciferase. Fluorescence from the two antibodies co-localization to the plasma membrane. By contrast, staining of cells transfected with RHO (where the human rhodopsin sequence had been cloned into the multiple cloning site of the Renilla luciferase 3′ UTR) showed luciferase localization within the cytosol and no rhodopsin signal (FIG. 15).

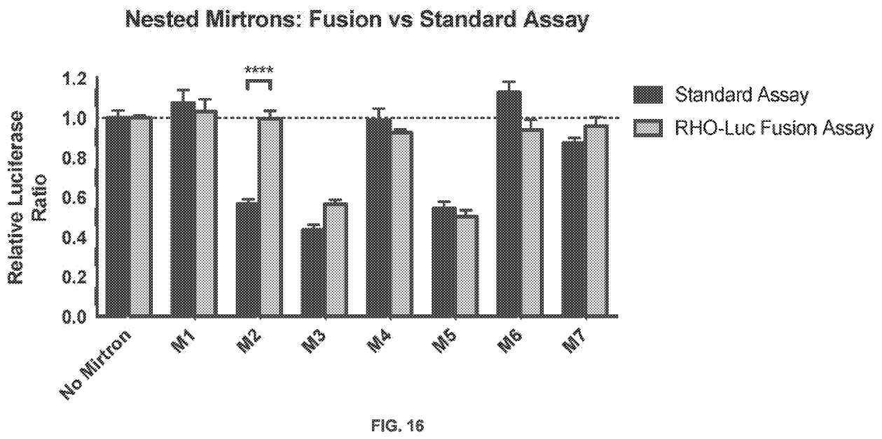

Having confirmed that the mirtron target sequences within RHO-Luc are indeed translated into protein, the plasmid was used to test mirtrons 1-7 in a further luciferase assay. Results of this fusion assay were comparable to that of the standard assay in all cases except for that of mirtron 2 which appeared ineffective when tested using the fusion assay (FIG. 16).

This supports the hypothesis that RNAi efficacy may, for some effectors, differ when a CDS rather than a 3′ UTR is targeted. This assay suggested that mirtrons 3 and 5 are likely to be the most effective of the seven candidate designs for downstream in vivo application. To our knowledge, this is the first time the Dual Luciferase assay has been applied to a fusion vector. This approach may be useful for in vitro testing of all RNA interference effectors that are designed to target coding sequences.

### Example 12—BLAST Search for Off-Target Homologous Sequences

If mirtrons are to be used for the treatment of patients with retinitis pigmentosa, it is important that off-target effects within transduced rods are minimal. The data presented above for M5H and M5M would suggest that small differences in the target sequence of mirtrons may not be well tolerated.

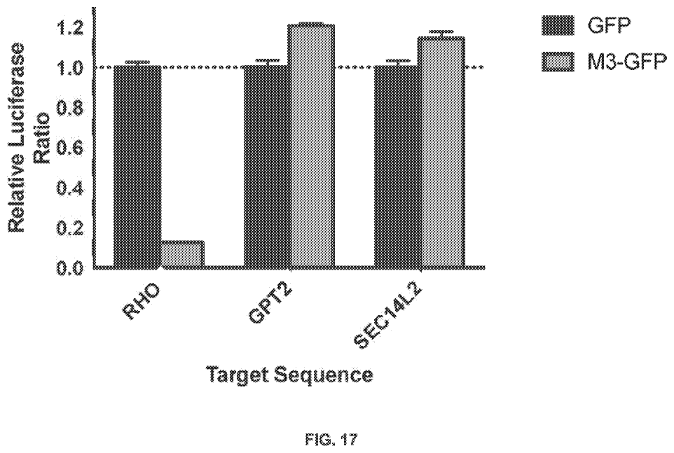

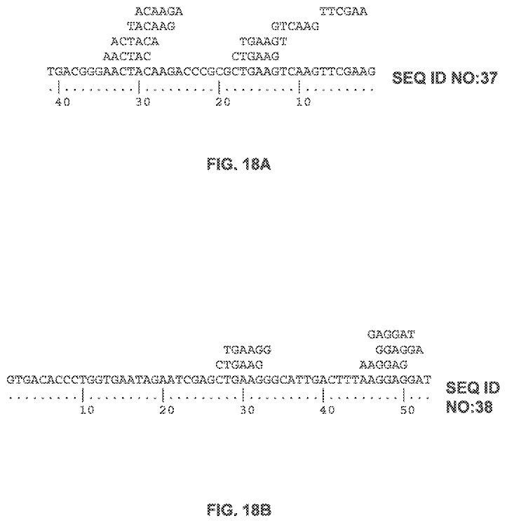

To investigate this further, the 21 bp target sequences for the three most effective mirtrons (2, 3 and 5) were subjected to a BLAST search (www.endembl.org) for homology within the mouse and human transcriptomes. All matches with an E-value (the calculated probability of the alignment between the input and subject sequences being due to chance) of less than 10 were considered (Table 2). The longest match was an 18 bp complementary sequence (with one internal mismatch) within the GPT2 CDS (E-value=5.5). Several 14 bp alignments within the human transcriptome were also identified for mirtrons 3 and 5H which included a match between the M3 guide sequence and the 3′-UTR of the SEC14L2 gene (E-value=5.5). This potential target (along with GPT2) was explored further as nonsense mutations in SEC14L2 have been associated with a rare form of recessive retinitis pigmentosa, RP41/Stargardt disease type 4. To quantify the extent to which these predicted sequences are true off-targets for M3, the target sequence for each (along with 25 bp of upstream and downstream flanking DNA) was ligated into the multiple cloning site of the vector. The subsequent assay performed against mirtron 3 suggested that mRNA levels for GPT2 and SEC14L2 are not affected by the mirtron (FIG. 17).

TABLE 2
Potential off-targets of mirtrons. A BLAST search was performed of the human
and mouse transcriptomes with all targets with E-value <10 being considered)

| | | | | | Match | |
|---|---|---|---|---|---|---|
| | Human | Match (bp) | E-value | Mouse | (bp) | E-value |
| Mirtron 2 | RHO | 21 | 0.0004 | Rho | 21 | 0.0002 |
| | | | | Tenm2 | 14 | 3.7 |
| Mirtron 3 | RHO | 21 | 0.0004 | Rho | 21 | 0.0002 |
| | GPT2 | 18 (1 mismatch) | 5.5 | Ctsc | 14 | 3.7 |
| | IL34 | 14 | 5.5 | Lgals4 | 14 | 3.7 |
| | PTF1A | 14 | 5.5 | Tenm2 | 14 | 3.7 |
| | SEC14L2* | 14 | 5.5 | | | |
| | GOLGA8B | 14 | 5.5 | | | |
| | AKNA | 14 | 5.5 | | | |
| | INPL1 | 14 | 5.5 | | | |
| Mirtron 5H | RHO | 21 | 0.0004 | Rho | 21 | 0.0002 |
| | ZBTB10 | 14 | 5.5 | Ptprb | 15 | 0.94 |
| | RHPT1 | 14 | 5.5 | Tgm7 | 14 | 3.7 |
| | C11orf40 | 14 | 5.5 | Mtm | 14 | 3.7 |
| | | | | Mfsd8 | 14 | 3.7 |
| | | | | Ikbkap | 14 | 3.7 |

### Example 13—Insertion of Mirtrons and ESEs in the 5′UTR of a Reporter Gene

Mirtrons 2, 3 and 5 were cloned into the 5′-UTR of the CAG.GFP. WPRE plasmid. The inventors sought to promote efficient splicing of the mirtrons by also including additional sequences containing exonic splice enhancer (ESE) motifs immediately upstream and downstream of the mirtron as follows.

ESE's were identified in the sequences 5′ (SEQ ID NO: 37) and 3′ (SEQ ID NO: 38) of the mirtron insertion site (1 nucleotide downstream of the BstB1 restriction site) in the eGFP CDS (FIG. 18) using RESCUE-ESE (http://genes.mit.edu/burgelab/rescue-ese/). These sequences advantageously correspond to the sequences that flank the mirtron in the reporter plasmids used to determine the accuracy and efficiency of the mirtron splicing, as described above. This simplified the process of introducing both the mirtron and additional ESE(s) into the 5′UTR, and helps to maintain the splicing accuracy and efficiency already achieved in the reporter constructs (plasmids described in Example 5). An in silico splice analysis was performed using online software (Spliceport, http://spliceport.cbcb.umd.edu) to identify the optimal 5′ and 3′ flank length around the mirtron insertion site (Table 3). The longest 5′ and 3′ flanking sequences up to but excluding the next 5′ or 3′ ATG motifs (which may otherwise act as cryptic translational start sites) were selected to flank the mirtrons in the new constructs. The entire sequence (5′flank-mirtron-3′flank) was amplified from the reporter constructs and cloned into a restriction site within the 5′-UTR of the transgene, at an EcoRV blunt restriction site (GAT|ATC). This is ideal for the purpose as once the construct has been inserted, the EcoRV restriction site is reformed at the 3′ end. This allows multiple similar constructs (containing the same or different mirtrons) to be inserted sequentially in tandem (see Example 15 below).

TABLE 3
In silico splice analysis

| Upstream | Donor | Downstream | Acceptor |
|---|---|---|---|
| flank | site score | flank | site score |
| 0 | −1.01 | 0 | 2.41 |
| 5 | 0.8 | 5 | 3.02 |
| 10 | 0.59 | 10 | 2.65 |
| 15 | 0.4 | 15 | 2.51 |
| 20 | 1.07 | 20 | 2.32 |
| 25 | 1.21 | 25 | 2.26 |
| 30 | 0.92 | 30 | 2.1 |
| 35 | 1.56 | 35 | 2.47 |
| 41 | 1.73 | 40 | 2.27 |
| | | 45 | 2.06 |
| | | 50 | 2.35 |
| | | 53 | 2.55 |

Locating a “knock-down” mirtron in the 5′UTR of a “replacement” transgene has a number of potential advantages. Since the mirtron is upstream of the transgene start codon, expression of the transgene may be less disrupted than if the mirtron is located as an intron within the CDS. There is no risk of mutant protein production (provided that no ATG motifs are introduced into the 5′UTR that could act as alternative start codons) as the transgene CDS is not interrupted. Location of the mirtron in the 5′ UTR should also not increase nonsense mediated decay of the transgenic transcript, which may occur if the mirtron is located in the 3′UTR. Furthermore, locating the mirtron in the 5′UTR allows additional sequences including ESEs to be inserted adjacent to the mirtron to promote efficient and accurate splicing out of the mirtron without disrupting expression of the transgene.

### Example 14—Mirtrons in the 5′UTR are More Effective than Mirtrons in the CDS

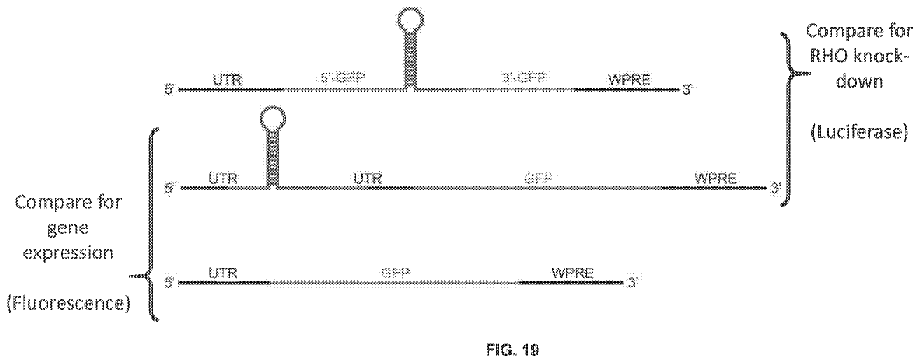

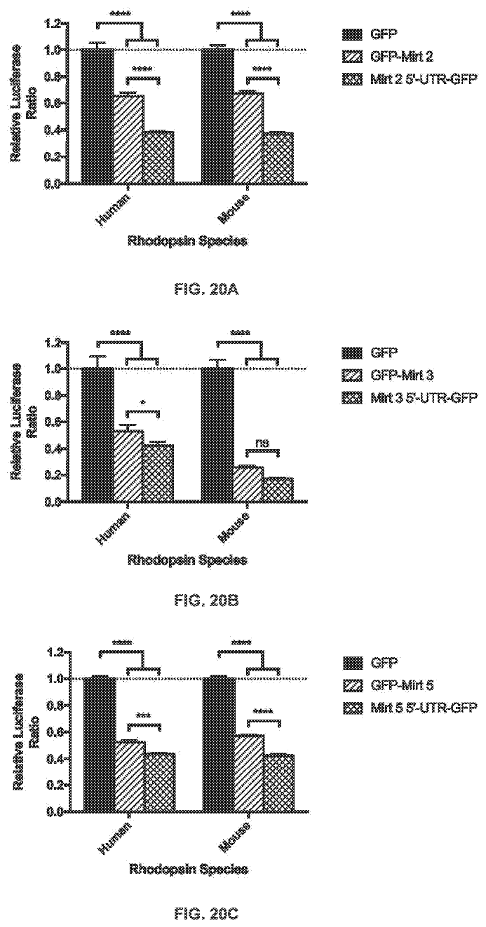

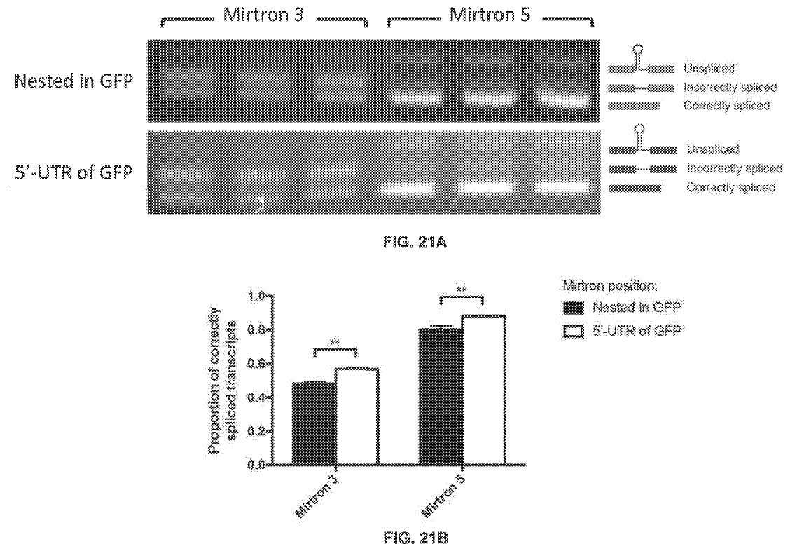

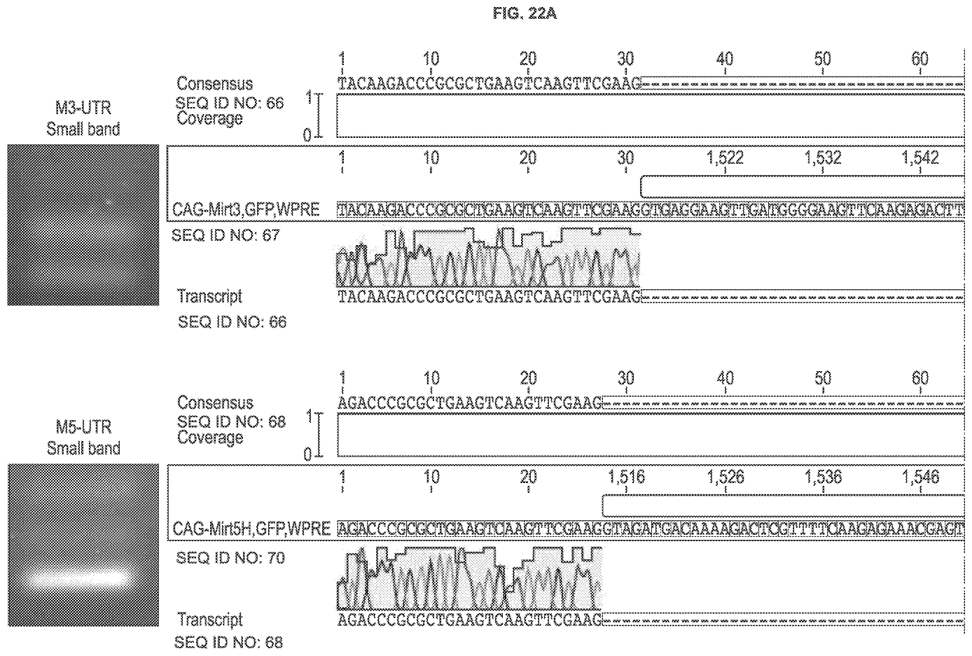

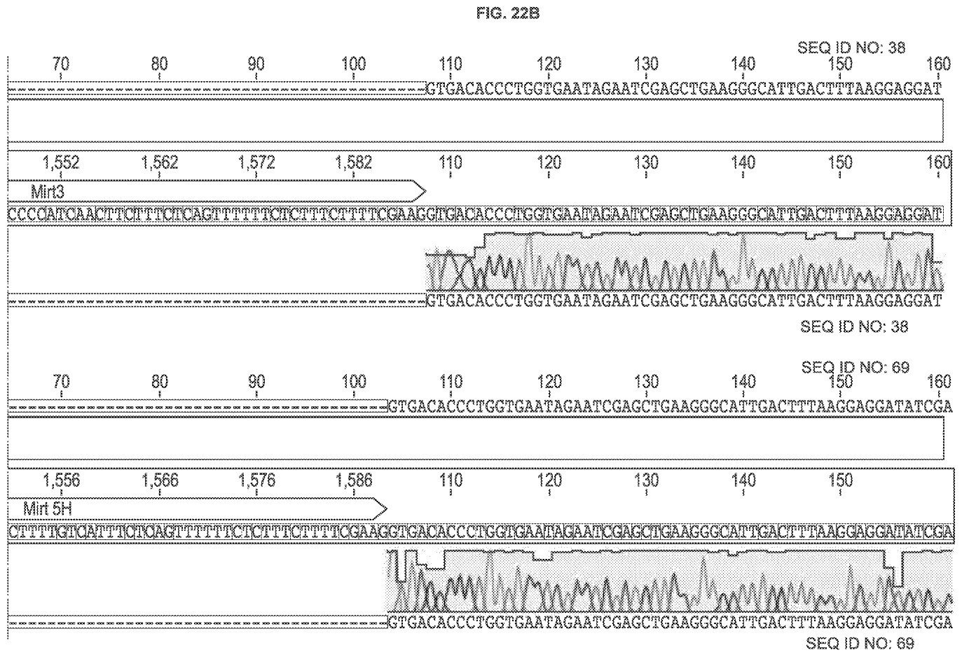

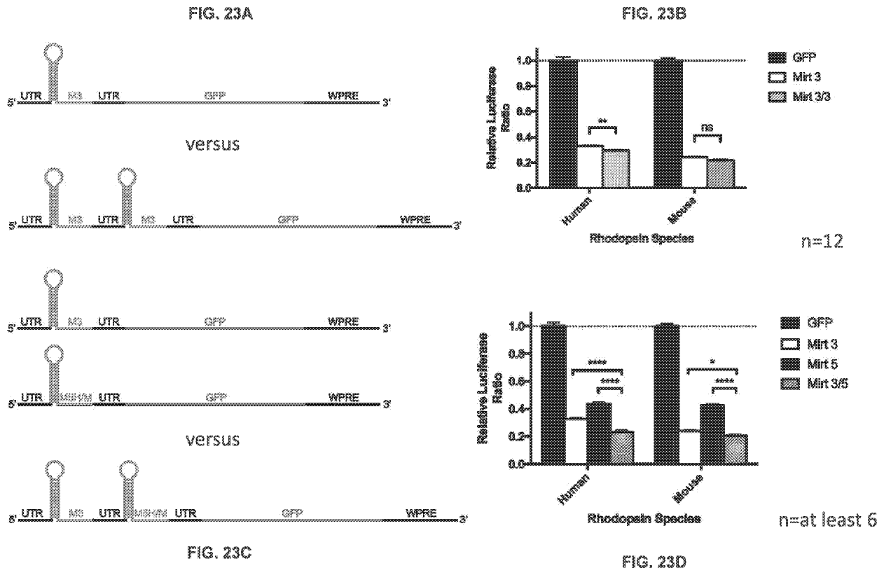

The new constructs, known as M2-UTR, M3-UTR and M5-UTR, were compared to the mirtronless GFP plasmid for rhodopsin knockdown using the dual luciferase assay, and for downstream gene expression by the fluorescence assay as described above (FIG. 19). Mirtrons were found to be more effective when located in the 5′-UTR than when nested inside the GFP coding sequence for both human and mouse rhodopsin targets (FIG. 20). A PCR splice analysis using cDNA from transfected cells as template was performed as described above. Band densitometry performed on the subsequent gel revealed that a greater proportion of transcripts were correctly spliced for 5′UTR mirtrons when compared with that of their CDS nested counterparts (FIG. 21). Confirmation that the lowest band did indeed represent correctly spliced mRNA was achieved by Sanger sequencing of extracted DNA (FIGS. 22A-22B).

### Example 15—Two Mirtrons in Tandem Knock Down Expression More Efficiently: Target Sequence in 3′UTR

Having established that mirtrons remain effective when spliced out of the 5′-UTR of the supplemental transcript, the effect of multiple mirtrons in tandem within the 5′-UTR was investigated. The following plasmids were cloned:
1. M3/M3-UTR: Two copies of M3 in series, each with their own ESE flanking sequences as described above, cloned into the 5′-UTR of the CAG.GFP.WPRE plasmid.
2. M3/M5-UTR: One copy of M3 and one copy of M5 in series, each with their own ESE flanking sequences as described above, cloned into the 5′-UTR of the CAG.GFP.WPRE plasmid.

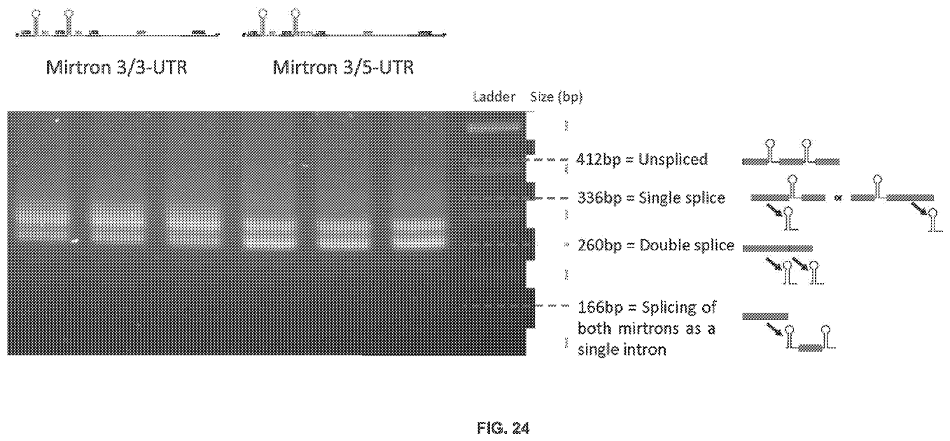

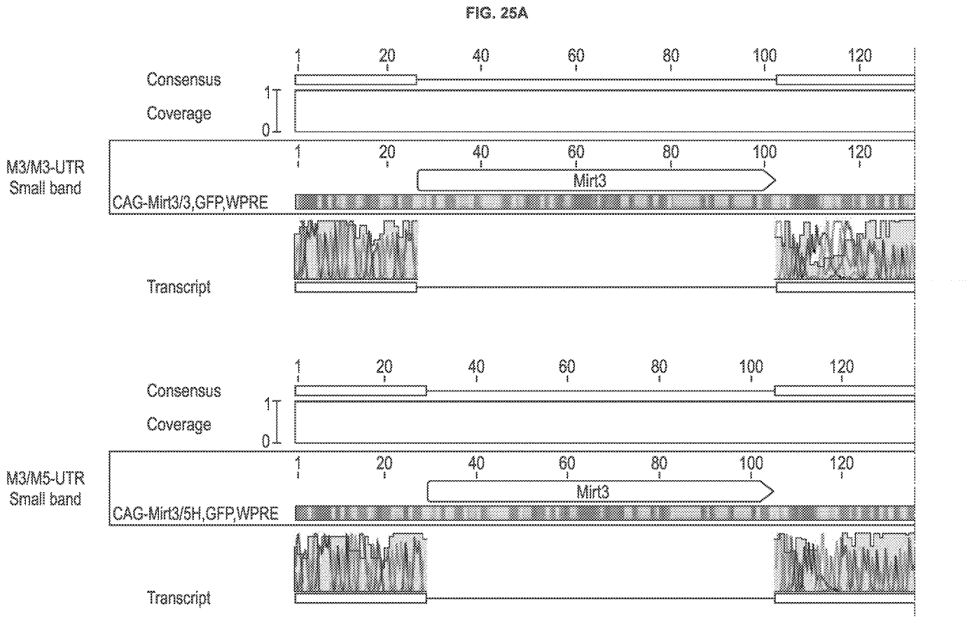

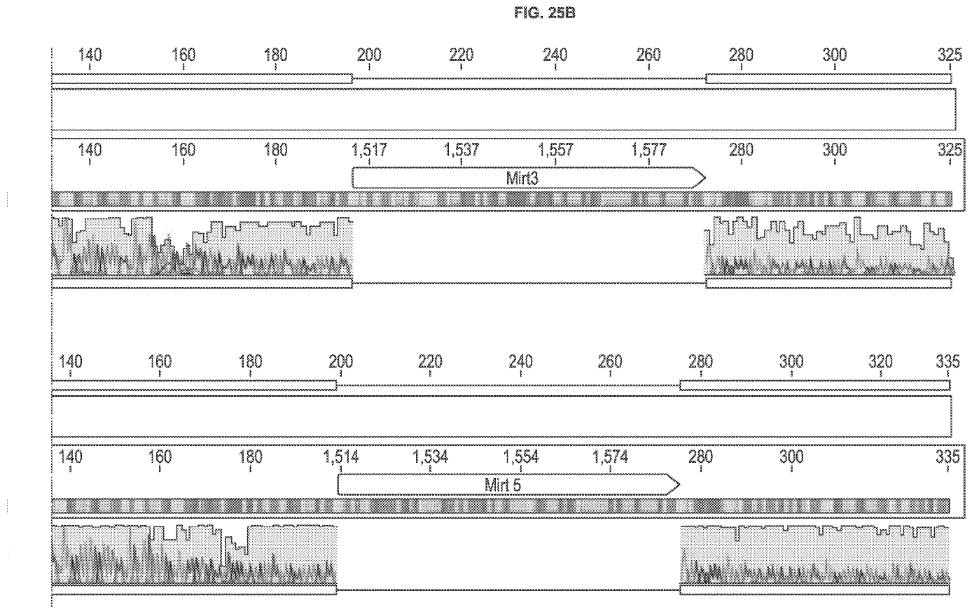

Comparison for knockdown of human and mouse rhodopsin against single 5′-UTR mirtrons using the dual luciferase assay showed superior efficacy of the tandem construct in both instances (FIG. 23). PCR splice analysis and Sanger sequencing confirmed that tandem mirtrons are independently and precisely spliced (FIGS. 24 & 25A-25B).

### Example 16—Two Mirtrons in Tandem Remain Effective when the Target Sequence is in the CDS

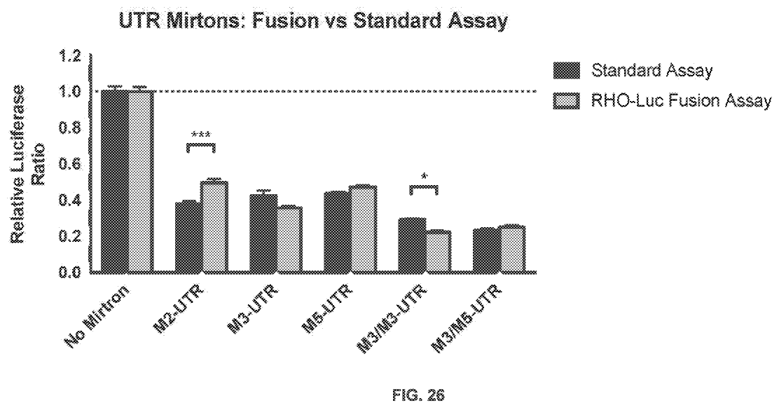

All 5′UTR mirtrons tested remained effective when directed against the RHO-Luciferase fusion protein described above (FIG. 26). Interestingly, the potency of M2-UTR was lower, and that of M3/M3-UTR was higher, in this assay than when measured using the standard dual luciferase assay.

### Example 17—Two Mirtrons in Tandem Slightly Reduce Reporter Gene Expression

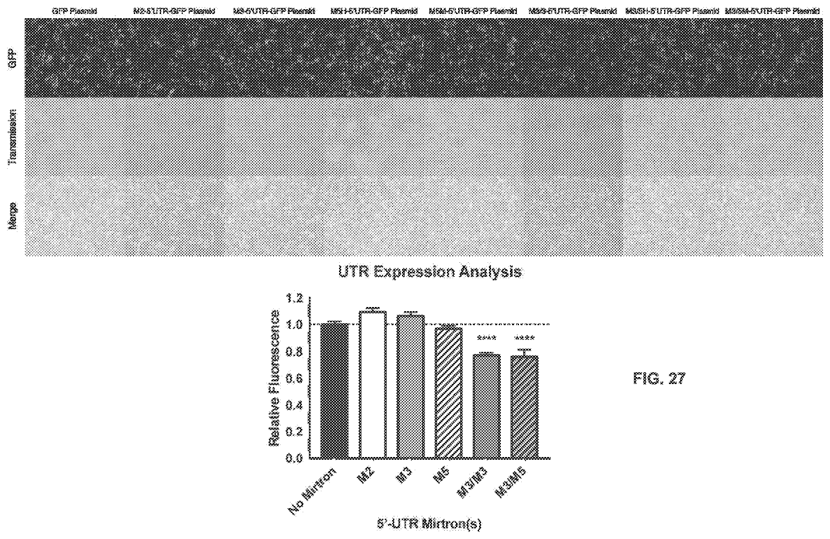

A fluorescence assay was performed on all 5′UTR mirtrons to elucidate their effect on downstream gene expression. FIG. 27 shows representative images of HEK293 cells transfected with these plasmids along with the results of a quantitative fluorescence assay performed on protein lysates from these cells. In summary, single 5′UTR mirtrons had no adverse effect on eventual protein levels whilst tandem mirtrons resulted in a small but significant reduction in reporter gene expression (23% and 24% reduction for M3/M3 and M3/M5 respectively).

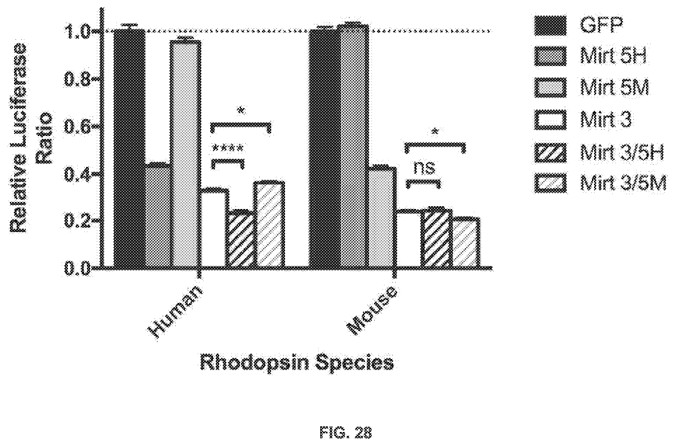

FIG. 28 shows the knock-down effect of M3-UTR, M5H-UTR, M5M-UTR, M3/M3-UTR, M3/M5H-UTR and M3/M5M against both human and mouse rhodopsin. Although M3 alone is effective in both species, the knock-down effect is significantly greater against the mouse transcript. For this reason, the combination of M3 with M5H was chosen for the block and replace vector as this combination achieved a similar level of efficient knockdown (>75%) in both species.

### Example 18—Design of Gene for “Replacement” Rhodopsin

For an effective ‘block and replace’ vector, the CDS of human rhodopsin included in the AAV construct must be resistant to co-expressed mirtrons. To achieve this, codons that constitute the target sites for mirtrons 3 and 5 were changed where possible to alternative sequences that encode the same amino acids (avoiding ‘rare’ codons). The resulting mRNA sequence will not be complementary to these mirtrons, should not be subject to degradation, but should nonetheless result in translation of normal human rhodopsin protein.

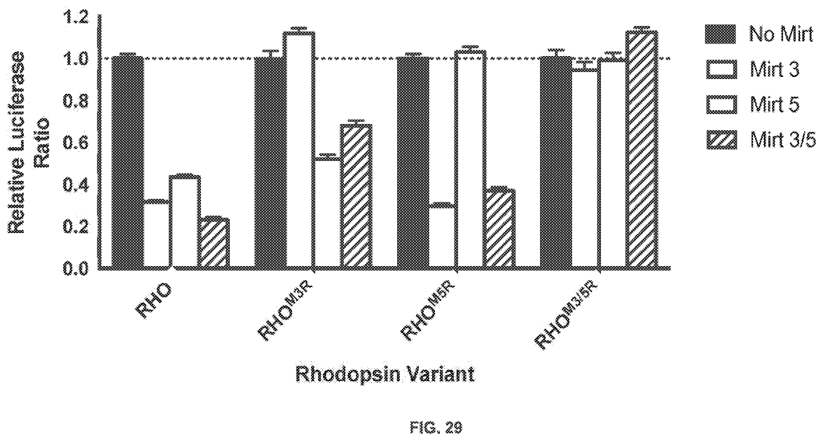

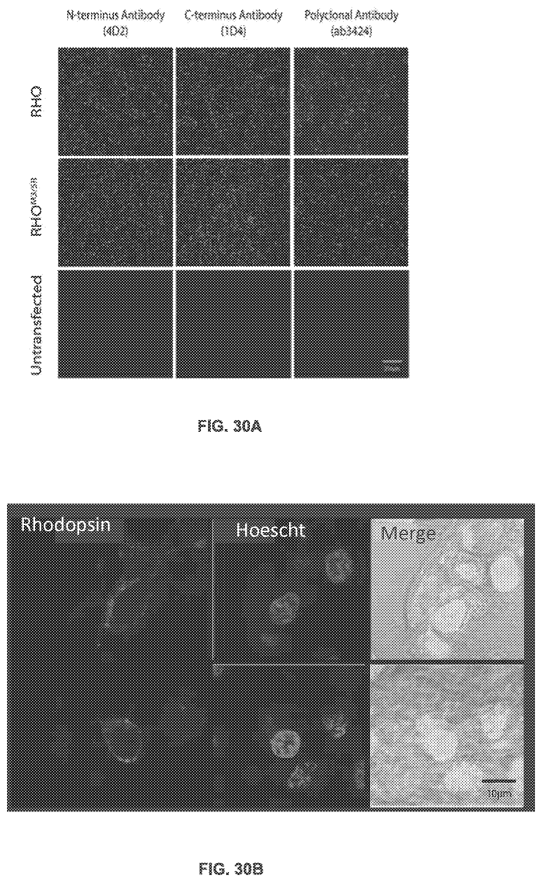

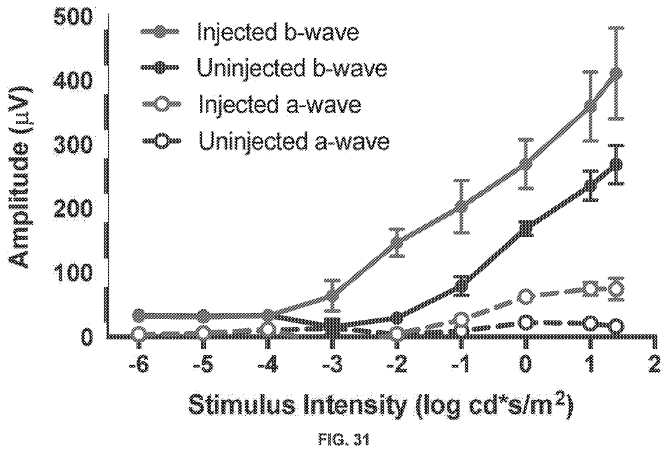

To test this, codon-modified versions of human rhodopsin resistant to M3 (RHO^{M3R}), M5 (RHO^{M5R}) and both (RHO^{M3/5R}) were individually cloned into the multiple cloning site of the vector. Resistance of these transcripts to degradation by their corresponding mirtrons was then established by the assay (FIG. 29). The target sequences of RHO (SEQ ID NO: 1) complemented by each guide strand and the corresponding sequence in “replacement” rhodopsin genes are as follows:

M3 target site in RHO (nucleotides 153-173):

(SEQ ID NO: 5)
CTTCCCCATCAACTTCCTCAC

M3 target site in RHO^{M3/5R} (nucleotides 153-173):

(SEQ ID NO: 24)
ATTTCCAATTAATTTTCTGAC

M5 target site in RHO (nucleotides 598-618):

(SEQ ID NO: 7)
ATCTACATGTTCGTGGTCCAC

M5 target site in RHO^{M3/5R} (nucleotides 598-618):

(SEQ ID NO: 25)
AATGAATCCTTCGTGATTTAT

### Example 20—RHO^{m3/5R} CDS Expresses Normally in HEK293 Cells

To confirm that the RHO^{M3/5R} CDS expresses normal rhodopsin protein, the sequence was cloned into a plasmid under the control of the CAG promoter with a 3′ WPRE (CAG.RHO^{M3/5R}.WPRE). This plasmid was then used to transfect HEK293 cells which were subsequently stained for rhodopsin by immunocytochemistry using three different antibodies. No difference in staining was observed in these cells compared to cells that had been transfected with a CAG.RHO.WPRE plasmid, and both versions trafficked to the plasma membrane (FIG. 30).

### Example 21-RHO^{m3/5R} CDS Expresses and Functions Normally In Vivo

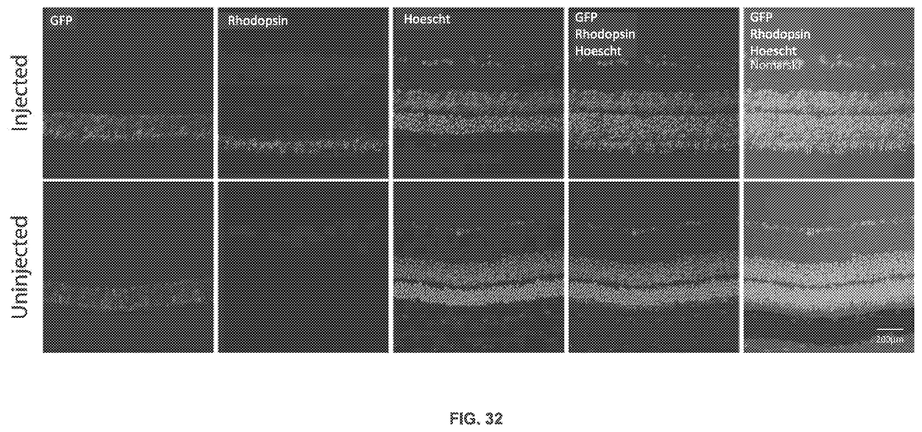

To test for appropriate expression and function of RHOM3/5R in vivo, 2×10⁹ gc of the vector in a total volume of 1.5 μl were injected subretinally into the right eyes of 10 Nrl.GFP/Rho^{-/-} mice. Four weeks later, dark-adapted electroretinography (ERG) was performed after which animals were sacrificed and cryosections of their eyes stained for rhodopsin by immunohistochemistry (IHC). A dark-adapted intensity-response curve (IRC) was constructed for injected right and uninjected left eyes which confirmed rescue of rod-derived responses of the electroretinogram (FIG. 31). IHC of retinal sections revealed presence of outer segments that were absent in uninjected eyes. These were packed with rhodopsin. No ectopic rhodopsin expression (i.e. outside of the outer segments) was identified in any case (FIG. 32). Thus, rhodopsin protein translated from an mRNA transcript that is resistant to both mirtrons 3 and 5 traffics to the outer segment of rods and is capable of driving the dark adapted light response.

### Example 22—Design and Manufacture of Vector “AAV-M3/5.RHO”

AAV2/8^{Y733F}.RHOp.Ex/Int.M3/M5.RHO^{M3/5R}.WPRE (henceforth referred to as AAV-M3/5.RHO) (SEQ ID NO: 31) was manufactured using a transgene plasmid cloned from the elements outlined above, including:
AAV genome: The AAV genome is flanked by inverted terminal repeat (ITR) sequences derived from AAV serotype 2.
Capsid: The capsid used is derived from AAV serotype 8 which has a predilection for photoreceptors, the target cell type for this treatment. The particular variant used is the Y733F mutant which has been shown to increase gene expression following viral transduction^{4,5}.
RHOp: The AAV genome uses the human rhodopsin promoter. This ensures that the virally delivered gene products are expressed only in rod photoreceptor cells.
Ex/Int: The first exon and intron of the chicken beta-actin gene together with the splice acceptor from the rabbit beta-globin gene is included 3′ of the RHO promoter. It was shown experimentally as discussed above that this improves downstream transgene expression whilst maintaining promoter cellular specificity.
M3/M5: Two 3′ tailed mirtrons (‘Mirtron 3’ and ‘Mirtron 5’) designed to target different regions of the human rhodopsin mRNA transcript;
DNA sequences containing exonic splice enhancers (ESEs) derived from the enhanced green fluorescent protein (eGFP) gene flanking and between the two mirtrons.
Kozak consensus sequence.
RHO^{M3/5R}: The human rhodopsin coding sequence (CDS). The 21 base pair target regions for mirtrons 3 and 5 have been altered using alternative codons such that the transcript is resistant to mirtron-mediated knock-down but nevertheless encodes the same functional protein, as described above.
WPRE: The Woodchuck Hepatitis Post-transcriptional Regulatory Element (WPRE) is included downstream of the RHO stop codon. This sequence has been shown to improve transgene expression following retinal gene therapy in mice and humans.
bovine growth hormone polyA signal.

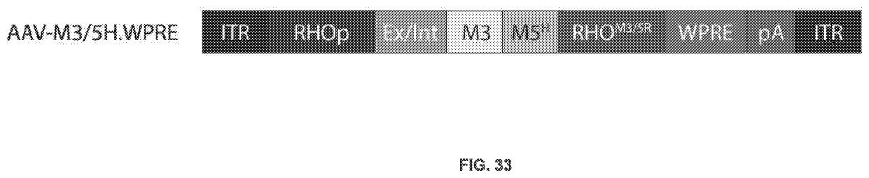

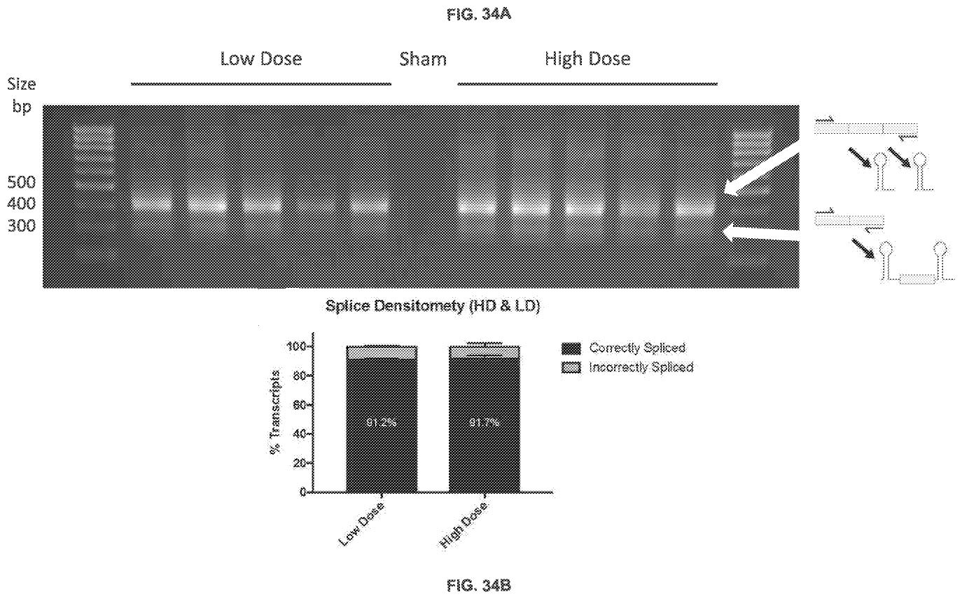

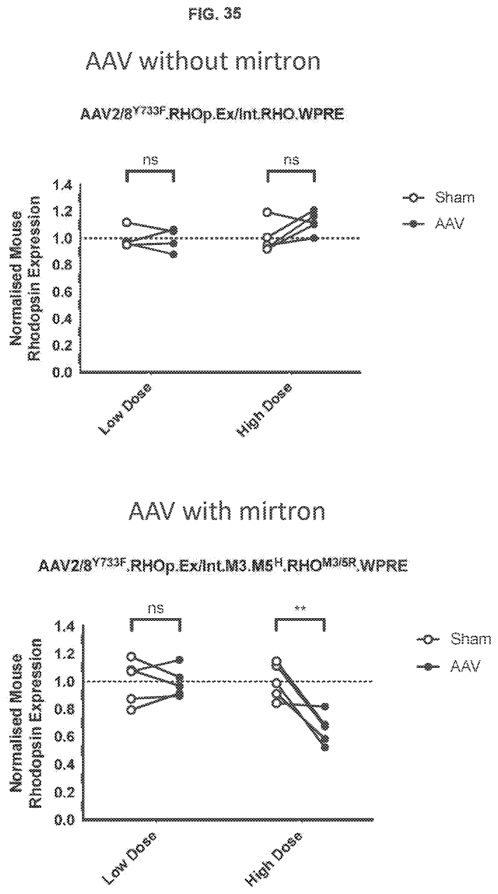

A schematic of AAV-M3/5.RHO is shown in FIG. 33.

### Example 23—In Vivo Experimental Design

To assess efficacy of AAV-M3/5.RHO as a treatment for RP caused by rhodopsin mutations, the vector was tested in P23H mice. The vector was delivered subretinally at a ‘low dose’ (n=17) of 2×10⁸ gc or a ‘high dose’ of 2×10⁹ gc in 1.5 μl into the right eyes of 3-week-old mice. Left eyes were not injected and acted as internal controls. Two further groups of mice were similarly injected with vector AAV-Ex/Int (see above), which is identical to AAV-M3/5.RHO but without the mirtrons and accompanying ESE-rich flanking sequences, and without RHO codon-modification.

A separate group of P23H mice (n=12) received sham subretinal injections of the 1.5 μl phosphate buffered saline (PBS) in the right eye. Again, left eyes were not injected. In all cases, injections were delivered superiorly.

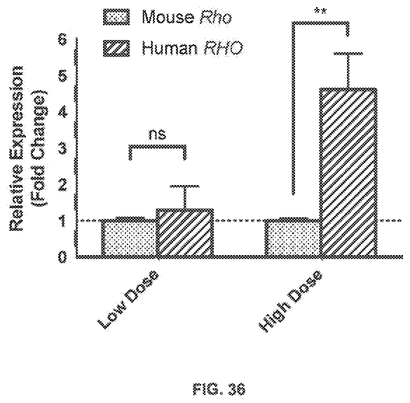

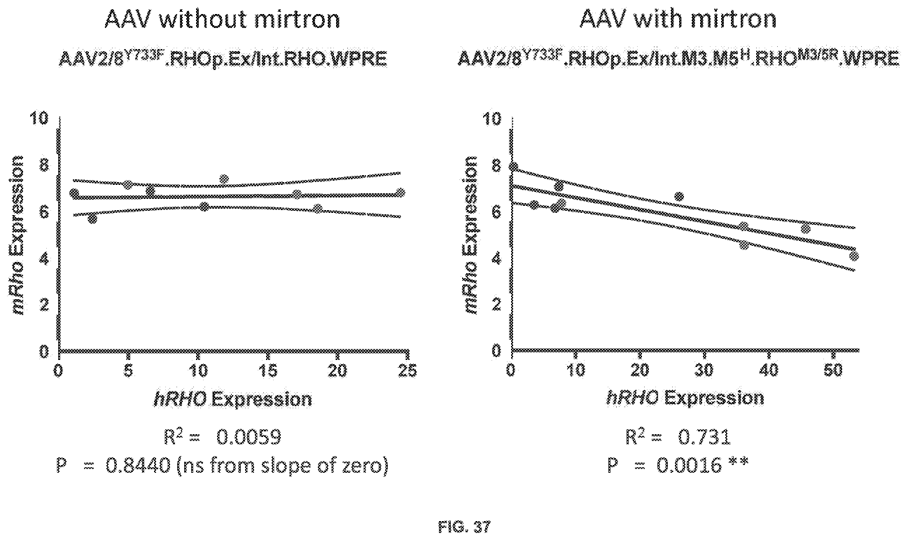

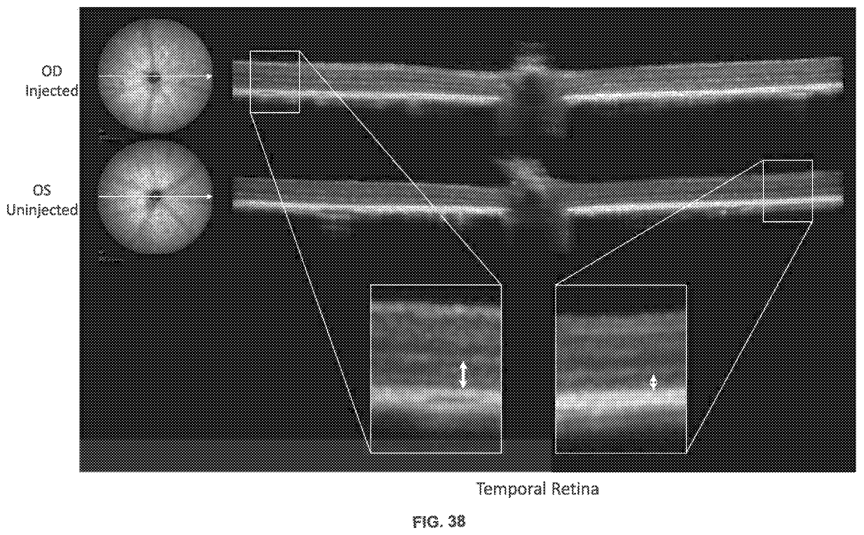

Retinae of some mice were extracted at 4 weeks post-injection for RNA extraction, cDNA synthesis and RT-qPCR (FIGS. 34 to 37). PCR splice analysis using mirtron-spanning primers and template cDNA derived from retinal lysates of injected mice showed highly efficient splicing in vivo (FIG. 34). For RT-qPCR, mouse and human rhodopsin in each eye was normalied to GFP levels (a gene only expressed in rods in P23H mice) to account for any rescue/toxicity effects. This data demonstrates that Mirtron 3 induces knock-down of mouse rhodopsin in vivo. No significant reduction in mRho was noted at low or high dose after delivery of AAV-Ex/Int which contains no mirtrons. When the mirtron-containing vector was delivered at high dose, a 34% knockdown in total retinal mRho mRNA was evident compared with fellow sham-injected eyes P=0.0024, 2-way ANOVA Sidak's multiple comparison test. No significant knockdown was seen at low dose (FIG. 35). At low dose, RHO supplementation approximates to native levels. At high dose, a 4.6 fold increase is seen, (P=0.0011, 2-way ANOVA Sidak's multiple comparison test) suggesting a degree of overexpression (FIG. 36). Human RHO expression correlated with mRho knockdown in eyes treated with the mirtron-containing vector but not with the AAV without Mirtron 3 (FIG. 37). Together, this gene expression analysis represents the first demonstration of function of an artificial mirtron in vivo.

For the remaining mice retinal anatomy has so far been assessed by SD-OCT at 1, 2 and 3 months post-injection and retinal function has been assessed by electroretinography (ERG) at 1 month and 3 months post-injection.

### Example 23—OCT Scans

Representative OCT scans from the low dose injected right and un-injected left eye of a treated P23H mouse at one month are shown in FIG. 38. Note that a thicker PRL is present in the treated eye (see arrows) which suggests a slowing of the retinal degeneration.

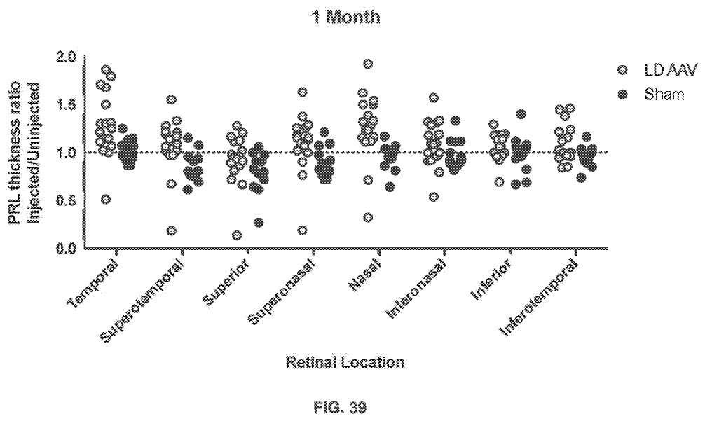

FIG. 39 shows photoreceptor layer (PRL) thickness of the injected eye normalised to that of its uninjected counterpart for each mouse in the AAV- and sham-injected groups. A small dip in PRL thickness can be appreciated superiorly in sham-injected animals (PRL ratio<1) as a result of surgery. In the AAV-injected group, this dip was not present and increased retinal thickness (PRL ratio>1) was evident along the horizontal meridian (p<0.0001 for both nasal and temporal retina, 2-way ANOVA, Sidak's multiple comparison test).

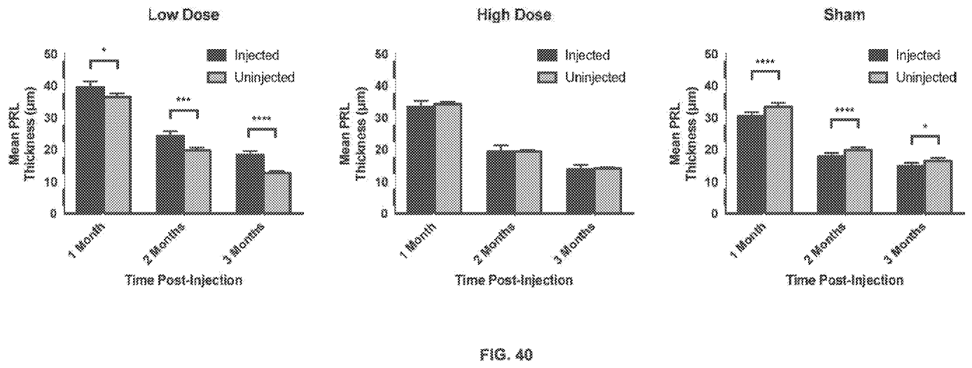

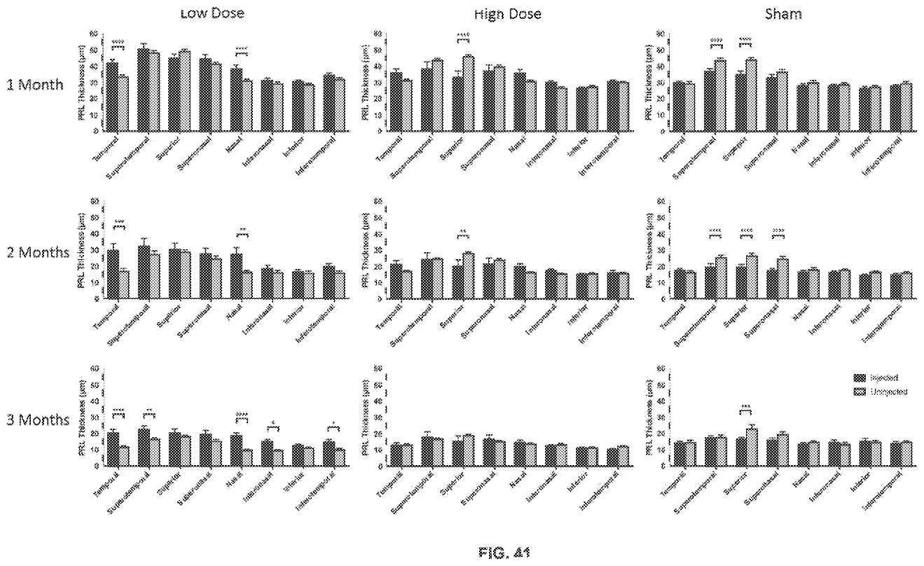

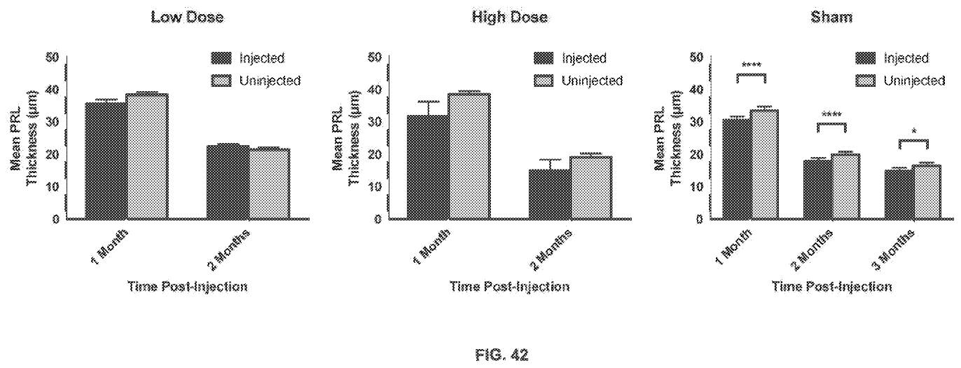

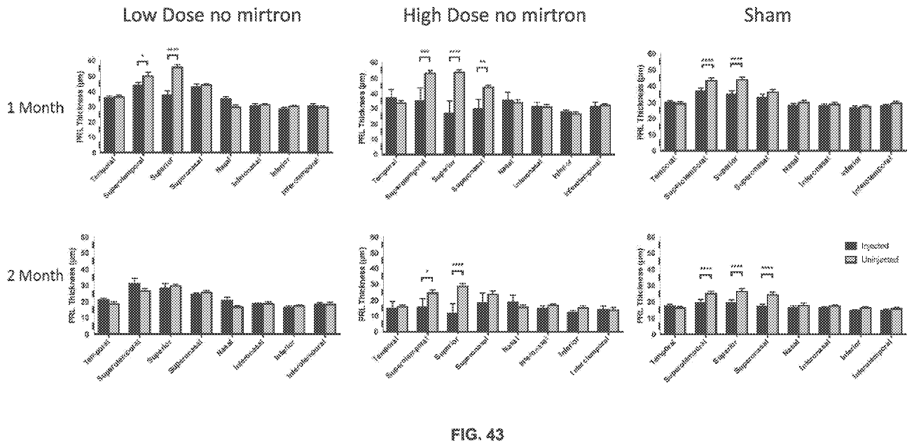

Further OCT analysis up to the 3 month time-point is shown in FIGS. 40 to 43.

### Example 25—Dark- and Light-Adapted ERG Analysis

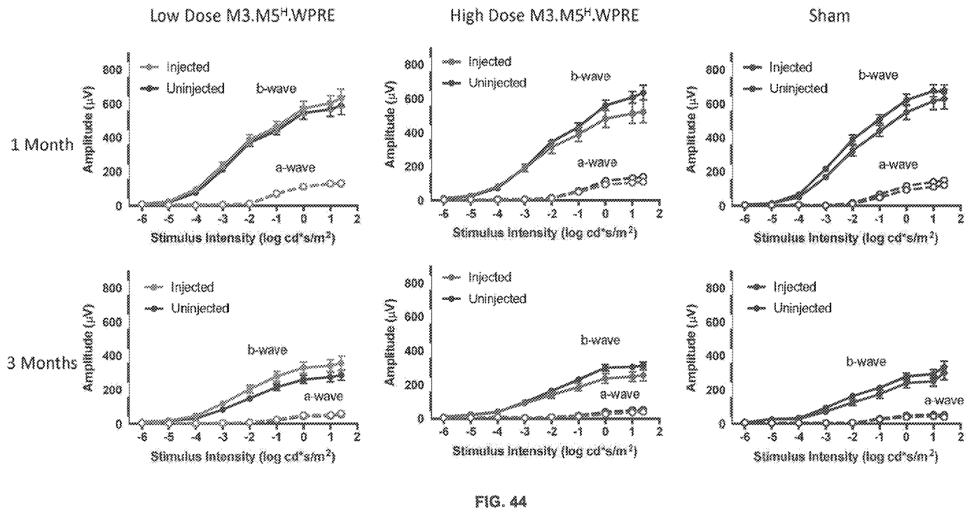

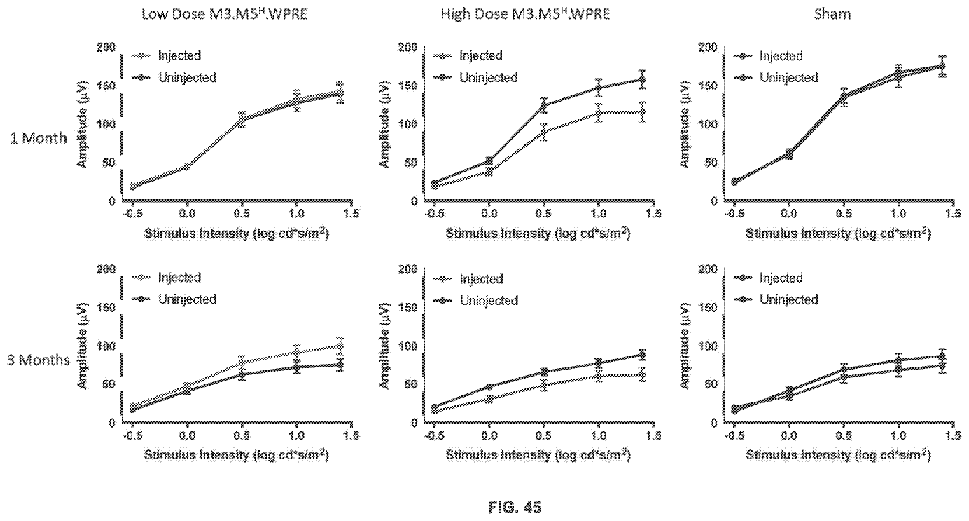

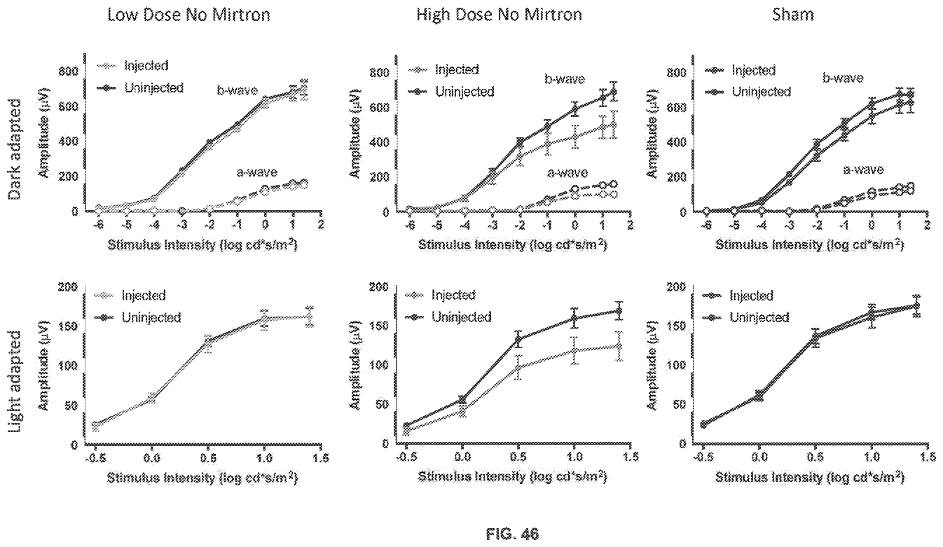

Dark- and light-adapted electroretinography analysis up to the 3 month time-point is shown in FIGS. 44 to 46.

### Example 26—Design and Manufacture of New Vectors without WPRE

The Examples above demonstrate that a benefit of AAV-M3/5^{H} in P23H mice is achieved using the low dose but some toxicity was apparent at high dose. Given that the mRNA analysis showed very high expression levels of RHO (see FIG. 36) and overexpression of this protein in known to be toxic to photoreceptors, it is likely that this is the cause. Mirtron expression is unlikely to be the cause as this toxicity was also seen with the mirtronless vector.

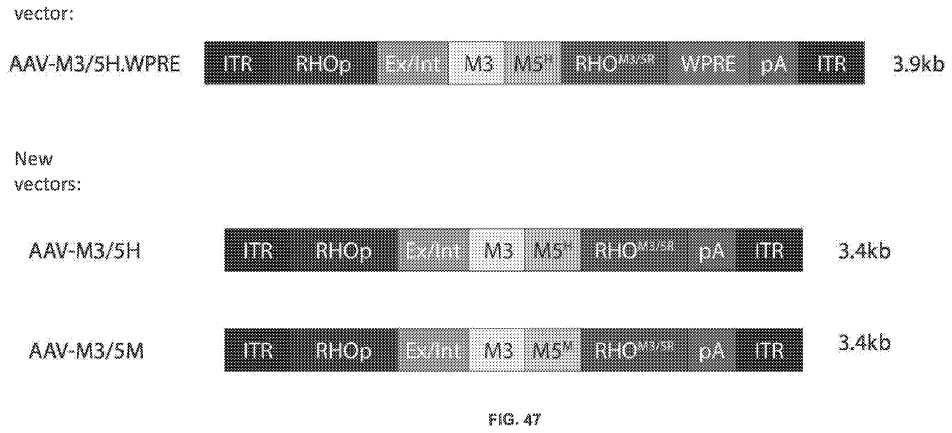

Two vectors were manufactured without the WPRE which should reduce RHO expression to more physiological levels. Schematic of the two vectors are shown in FIG. 47. Vector AAV2/8^{Y733F}.RHOp.Ex/Int.M3/M5H.RHO^{M3/5R} (“AAV-M3/5H”) contains the same two mirtrons as AAV2/8^{Y733F}.RHOp.Ex/Int.M3/M5.RHO^{M3/5R}.WPRE (AAV-M3/5.RHO), whereas AAV2/8^{Y733F}.RHOp.Ex/Int.M3/M5M.RHO^{M3/5R} (“AAV-M3/5M”) replaces the mirtron 5 directed against human RHO with the mirtron 5 directed against mouse RHO. This will make it possible to assess the difference between having one or two active mirtrons in vivo.

### Example 27—Triple Mirtron Vectors

To achieve a safe and therapeutic balance between knockdown of endogenous rhodopsin (mutant and wild-type) and replacement mirtron-resistant rhodopsin (codon-modified), double and triple mirtron vector designs were compared. The double mirtron vector represents the original design, containing mirtron M3 that can bind to mouse and human rhodopsin and mirtron M5, which can only bind to human rhodopsin. The triple mirtron vector contains M3 and M5 plus an additional copy of M3 (the 5′UTR of the triple mirtron vector is set forth in SEQ ID NO: 72, see Example 28 below).

### Testing in Human-Derived Retinal Organoids

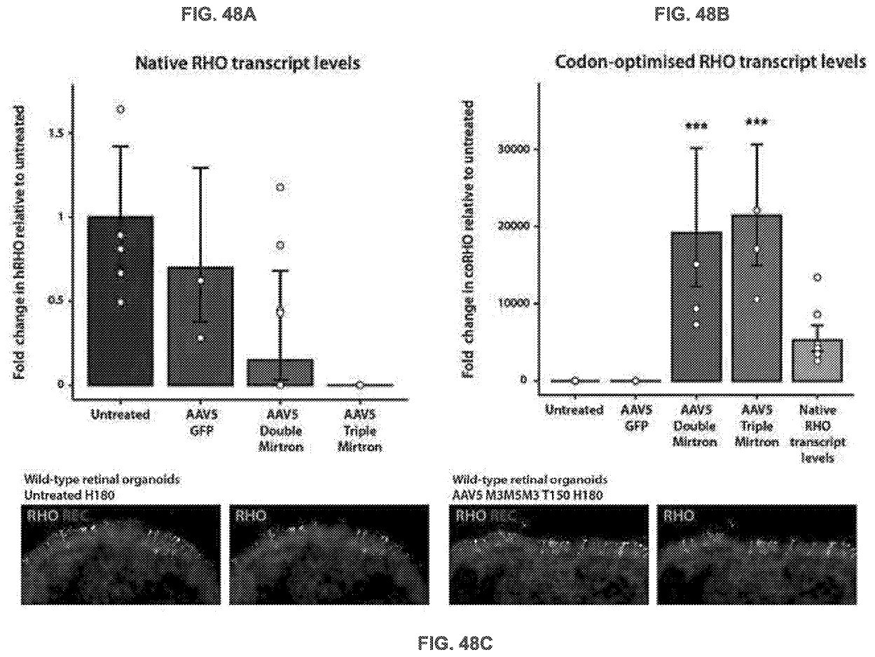

Retinal organoids are an advanced in vitro model of the retina and the double and triple mirtron vectors were initially applied to wild-type retinal organoids. Assessment of rhodopsin transcripts by RT-qPCR identified that more consistent, complete knockdown of endogenous rhodopsin was achieved with the triple mirtron vector compared to the double mirtron vector (FIG. 48A). Both the double and triple mirtron vectors enabled similar levels of overexpression of codon-modified rhodopsin (FIG. 48B). In triple mirtron vector treated wild-type retinal organoids, staining of rhodopsin protein showed a comparable pattern to that observed in untreated wild-type retinal organoids (FIG. 48C).

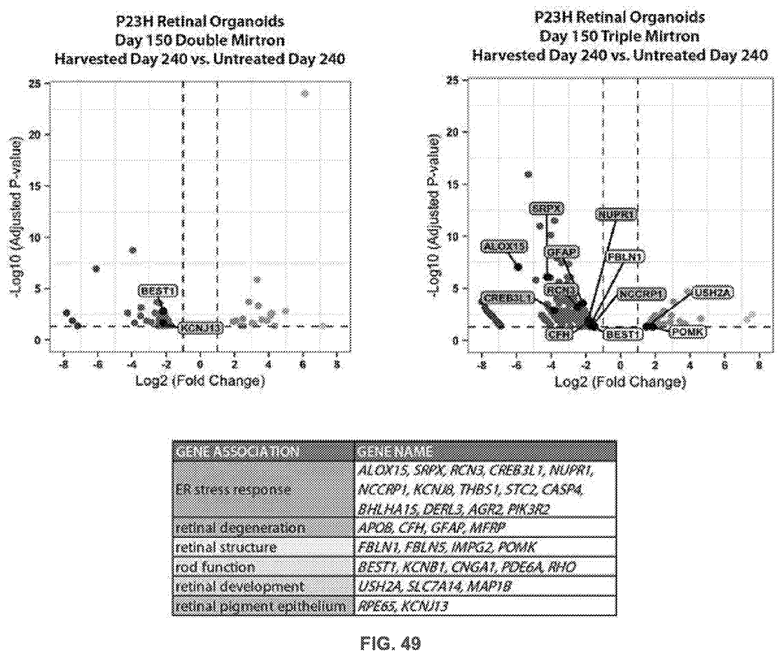

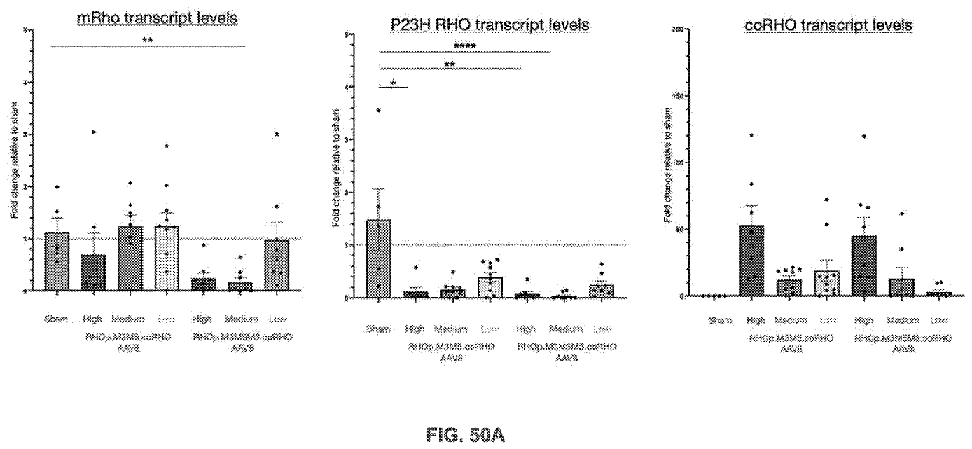

Retinal organoids were generated from a patient carrying the RHO P23H mutation and treated with the double or triple mirtron vector. Bulk RNAseq transcriptome analysis identified differential gene expression of genes linked to endoplasmic reticulum stress, retinal degeneration, retinal structure, rod function, retinal development and the retinal pigment epithelium (FIG. 49). The triple mirtron vector enabled greater changes in these transcriptome profiles compared to untreated P23H retinal organoids, indicating signs of a treatment effect not observed to the same extent with the double mirtron vector.

### Testing in Humanised Mouse Models

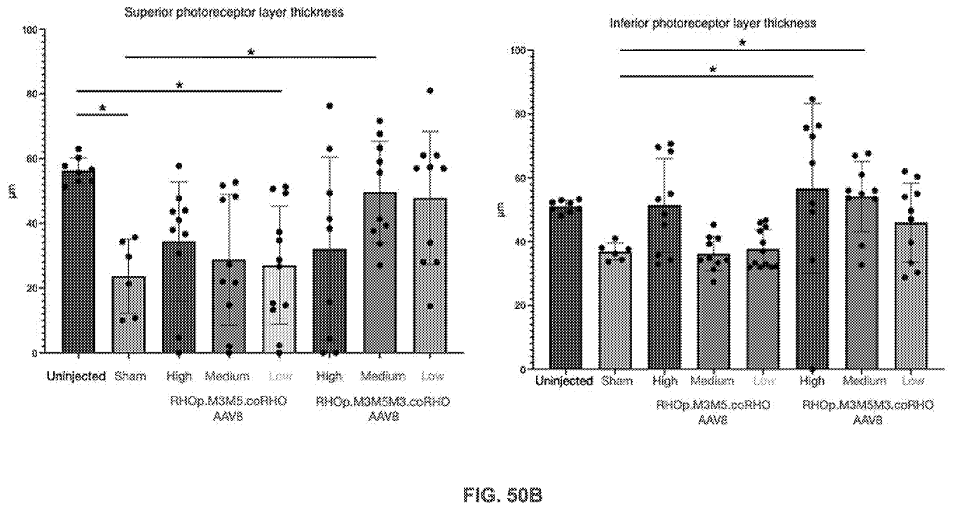

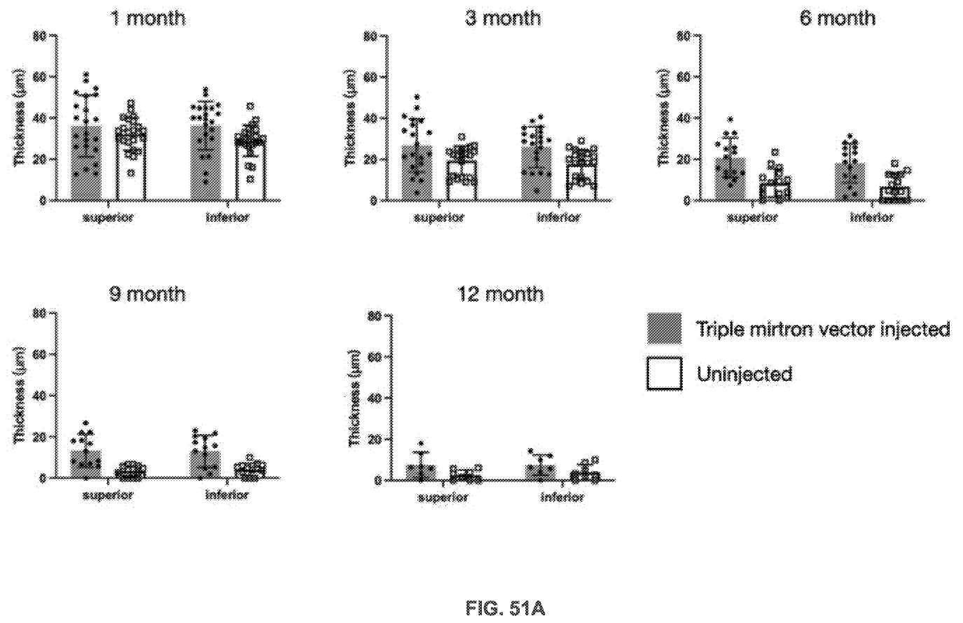

The humanised P23H mouse model contains an insertion of the human rhodopsin gene carrying the P23H mutation (copy number unknown). To avoid rhodopsin toxicity/overexpression, experimental mice were achieved by crossing the human P23H knock-in with rhodopsin knockout mice. A preliminary study was performed in these Rho^{+/-,P23H+} mice, performing a subretinal injection at 3-4 weeks of age with different doses of the double or triple mirtron vector (FIGS. 50A-50B). Both vectors enabled supplementation of codon-modified rhodopsin, but knockdown of endogenous rhodopsin was more effective with the triple mirtron vector (FIG. 50A). In this pilot study, evidence of retinal structure preservation was apparent compared to sham injected eyes with the triple mirtron vector (FIG. 50B). In the superior retina where the injection was performed, some retinal structure was lost with the high dose but this was not observed with the medium or low dose.

The comparisons of the double and triple mirtron vectors in human-derived retinal organoids and in the humanised P23H mouse model indicated that the triple mirtron vector outperformed the double mirtron vector. The triple mirtron vector enabled more complete knockdown of endogenous rhodopsin with balanced levels of replacement codon-modified rhodopsin. Long term in vivo studies were subsequently performed with the triple mirtron vector.

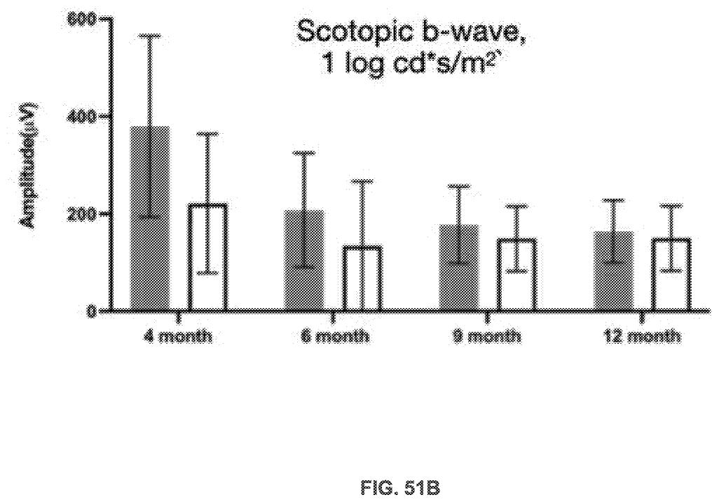

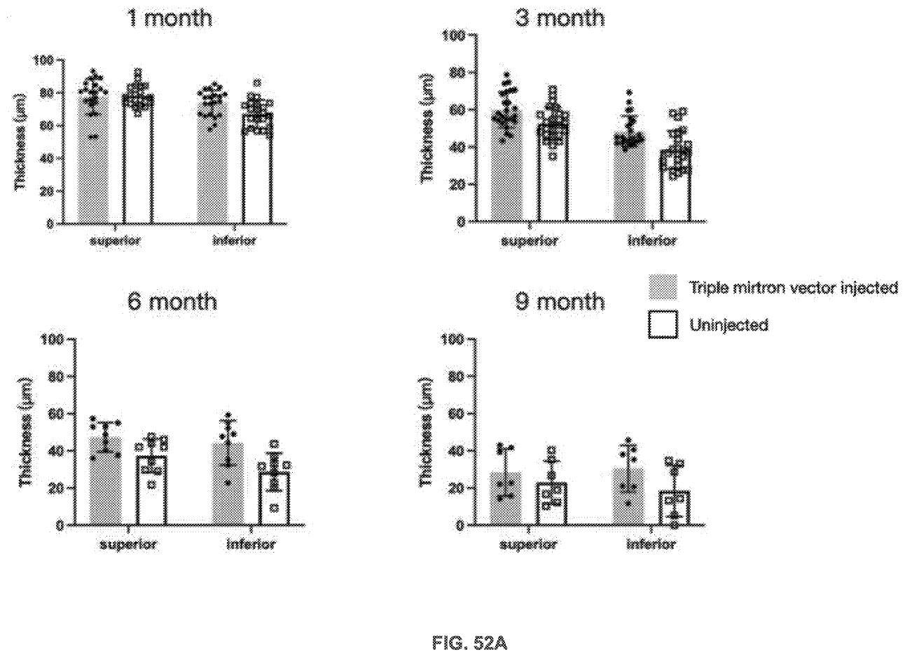

A new cohort of Rho^{+/-,P23H+} mice were injected in one eye at 3-4 weeks of age with 5E+08 genome copies/eye of the triple mirtron vector, the paired eye was left uninjected. At 1—, 3—, 6—, 9- and 12-months post-injection, preservation of the photoreceptor layer in the injected eye was apparent compared to the paired uninjected eye (FIG. 51A). This coincided with improved scotopic electroretinography (ERG) responses in treated eyes (FIG. 51B). Significant knockdown of endogenous rhodopsin transcripts and concomitant expression of codon-modified rhodopsin was observed at all time points (FIG. 51C).

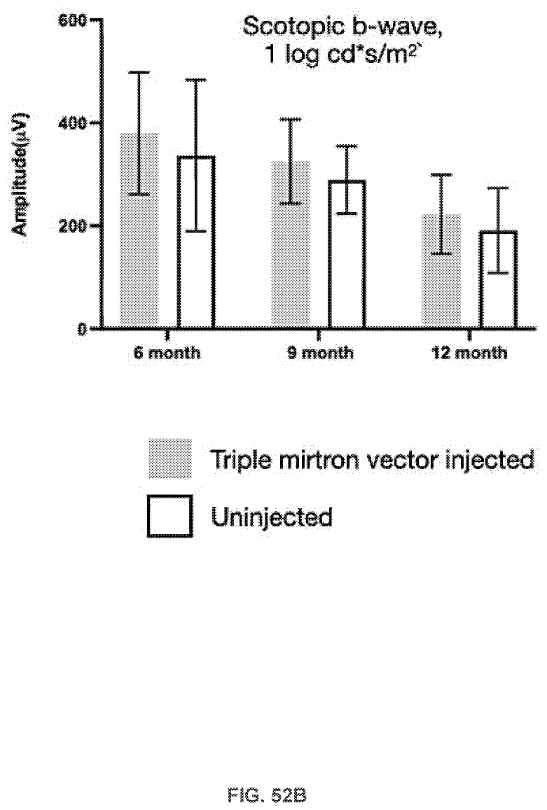

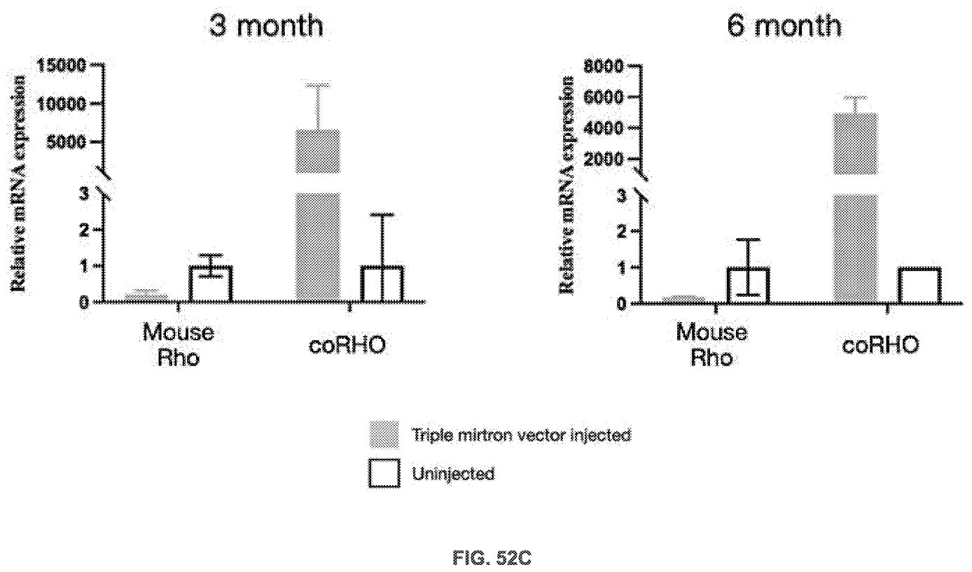

The Rho^{+/C110R} mouse model contains a humanised rhodopsin exon 1 carrying the C110R mutation, which causes a slow retinal degeneration. Rho^{+/C110R} mice received triple mirtron vector in one eye with the paired eye left uninjected. At 1—, 3—, 6- and 9-months post-injection, photoreceptor layer thickness was consistently greater in the superior and inferior retina of triple mirtron vector treated eyes (FIG. 52A). At 6—, 9- and 12-months post-injection, scotopic ERG responses were stronger in treated eyes compared to uninjected eyes (FIG. 52B). Endogenous rhodopsin was significantly knocked down in triple mirtron vector treated eyes with codon-modified rhodopsin strongly detected (FIG. 52C).

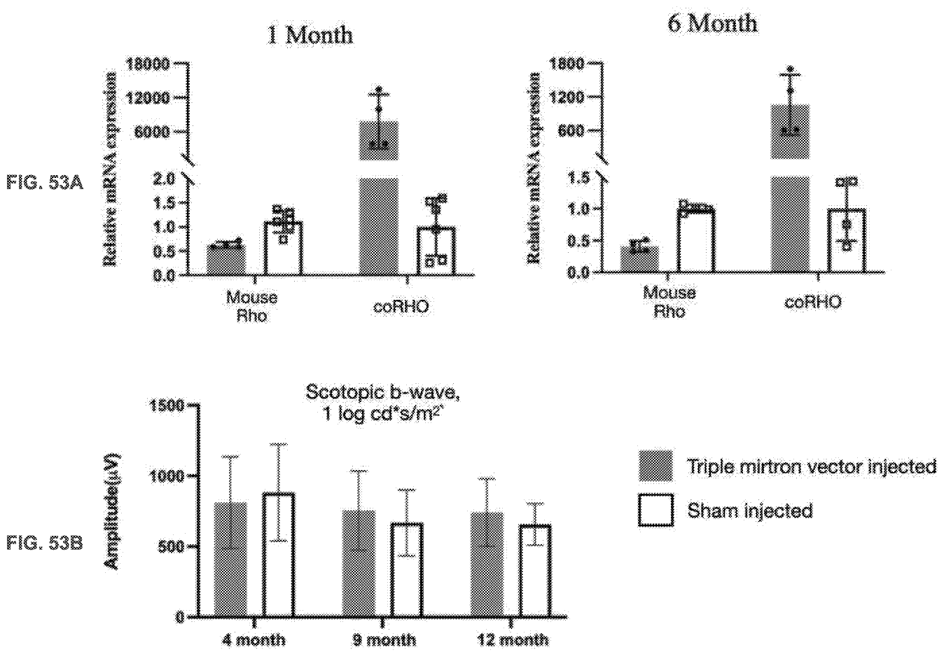

To assess for vector safety and confirm that mirtron knockdown of endogenous rhodopsin is balanced with replacement levels of codon-modified rhodopsin, a cohort of wild type mice received the triple mirtron vector in one eye with the paired eye receiving a sham injection. At 1- and 6-months post-injection, strong expression of the codon-modified rhodopsin was observed with ~50% knockdown of endogenous rhodopsin in triple mirtron injected eyes (FIG. 53A). Scotopic ERG responses were comparable between paired eyes, with triple mirtron vector eyes showing mildly increased responses compared to paired sham injected eyes at 9- and 12-months post-injection (FIG. 53B).

In summary, whilst both the double mirtron vector and the triple mirtron vector demonstrate a therapeutic effect, the triple mirtron achieves an improvement in the desired balance of endogenous rhodopsin knockdown with concomitant replacement of mirtron-resistant codon-modified rhodopsin. By including a second copy of M3, the triple mirtron vector enabled significant and sustained knockdown of endogenous rhodopsin in retinal organoids and two humanised mouse models of rhodopsin-related disease. Evidence of safety was achieved in wild-type mice with indications of efficacy shown by changes in gene expression profiles in patient-derived retinal organoids and by improved structure and function phenotype in two humanised mouse models of rhodopsin-related disease.

### Example 28—Additional Sequences of the Present Disclosure

SEQ ID NO: 72 sets forth a triple mirtron vector which contains M3 and M5 plus an additional copy of M3, where the 5′UTR of the rhodopsin transgene consists of a 5′ end consisting of the sequence of nucleotides 1 to 423 of SEQ ID NO: 39; a 3′ end consisting of the sequence of nucleotides 424 to 439 of SEQ ID NO: 39; and a sequence inserted between nucleotides 1 to 423 and 424 to 439 of SEQ ID NO: 39; where the inserted sequence consists of, in a 5′ to 3′ direction: (a) SEQ ID NO: 26, (b) ATC, (c) SEQ ID NO: 37, (d) SEQ ID NO 12, and (e) SEQ ID NO: 38.

SEQ ID NO: 72:
CACATACGATTTAGGTGACACTATAGAATACACGGAATTAATTCTAGCTGCGC
GCTCGCTCGCTCACTGAGGCCGCCCGGGCAAAGCCCGGGCGTCGGGCGACCTT
TGGTCGCCCGGCCTCAGTGAGCGAGCGAGCGCGCAGAGAGGGAGTGGCCAAC
TCCATCACTAGGGGTTCCTTGTAGTTAATGATTAACCCGCCATGCTACTTATCT
ACGTAGCCATGCTCTAGGTACCCAGATCTTCCCCACCTAGCCACCTGGCAAAC
TGCTCCTTCTCTCAAAGGCCCAAACATGGCCTCCCAGACTGCAACCCCCAGGC
AGTCAGGCCCTGTCTCCACAACCTCACAGCCACCCTGGACGGAATCTGCTTCTT
CCCACATTTGAGTCCTCCTCAGCCCCTGAGCTCCTCTGGGCAGGGCTGTTTCTT
TCCATCTTTGTATTCCCAGGGGCCTGCAAATAAATGTTTAATGAACGAACAAG
AGAGTGAATTCCAATTCCATGCAACAAGGATTGGGCTCCTGGGCCCTAGGCTA
TGTGTCTGGCACCAGAAACGGAAGCTGCAGGTTGCAGCCCCTGCCCTCATGGA
GCTCCTCCTGTCAGAGGAGTGTGGGGACTGGATGACTCCAGAGGTAACTTGTG
GGGGAACGAACAGGTAAGGGGCTGTGTGACGAGATGAGAGACTGGGAGAATA
AACCAGAAAGTCTCTAGCTGTCCAGAGGACATAGCACAGAGGCCCATGGTCCC
TATTTCAAACCCAGGCCACCAGACTGAGCTGGGACCTTGGGACAGACAAGTCA
TGCAGAAGTTAGGGGACCTTCTCCTCCCTTTTCCTGGATCCTGAGTACCTCTCC
TCCCTGACCTCAGGCTTCCTCCTAGTGTCACCTTGGCCCCTCTTAGAAGCCAAT
TAGGCCCTCAGTTTCTGCAGCGGGGATTAATATGATTATGAACACCCCCAATCT
CCCAGATGCTGATTCAGCCAGGAGCTTAGGAGGGGGAGGTCACTTTATAAGGG
TCTGGGGGGGTCAGAACCCAGAGTCATCCGGAGTCGCTGCGCGCTGCCTTCGC
CCCGTGCCCCGCTCCGCCGCCGCCTCGCGCCGCCCGCCCCGGCTCTGACTGACC
GCGTTACTCCCACAGGTGAGCGGGCGGGACGGCCCTTCTCCTCCGGGCTGTAA
TTAGCGCTTGGTTTAATGACGGCTTGTTTCTTTTCTGTGGCTGCGTGAAAGCCTT
GAGGGGCTCCGGGAGGGCCCTTTGTGCGGGGGGAGCGGCTCGGGGCTGTCCGC
GGGGGGACGGCTGCCTTCGGGGGGGACGGGGCAGGGCGGGGTTCGGCTTCTG
GCGTGTGACCGGCGGCTCTAGAGCCTCTGCTAACCATGTTCATGCCTTCTTCTT
TTTCCTACAGCTCCTGGGCAACGTGCTGGTTATTGTGCTGTCTCATCATTTTGGC
AAAGAATTGGATCCTAGCTTGATTGACGGGAACTACAAGACCCGCGCTGAAGT
CAAGTTCGAAGGTGAGGAAGTTGATGGGGAAGTTCAAGAGACTTCCCCATCAA
CTTCTTTCTCAGTTTTTTCTCTTTCTTTTCGAAGGTGACACCCTGGTGAATAGAA
TCGAGCTGAAGGGCATTGACTTTAAGGAGGATTGACGGGAACTACAAGACCCG
CGCTGAAGTCAAGTTCGAAGGTAGATGACAAAAGACTCGTTTTCAAGAGAAAC
GAGTCTTTTGTCATTTCTCAGTTTTTTCTCTTTCTTTTCGAAGGTGACACCCTGG
TGAATAGAATCGAGCTGAAGGGCATTGACTTTAAGGAGGATATCTGACGGGAA
CTACAAGACCCGCGCTGAAGTCAAGTTCGAAGGTGAGGAAGTTGATGGGGAA
GTTCAAGAGACTTCCCCATCAACTTCTTTCTCAGTTTTTTCTCTTTCTTTTCGAA
GGTGACACCCTGGTGAATAGAATCGAGCTGAAGGGCATTGACTTTAAGGAGGA
TATCGAATTCCGCCACCATGAATGGCACAGAAGGCCCTAACTTCTACGTGCCC
TTCTCCAATGCGACGGGTGTGGTACGCAGCCCCTTCGAGTACCCACAGTACTA
CCTGGCTGAGCCATGGCAGTTCTCCATGCTGGCCGCCTACATGTTTCTGCTGAT
CGTGCTGGGATTTCCAATTAATTTTCTGACGCTCTACGTCACCGTCCAGCACAA
GAAGCTGCGCACGCCTCTCAACTACATCCTGCTCAACCTAGCCGTGGCTGACC
TCTTCATGGTCCTAGGTGGCTTCACCAGCACCCTCTACACCTCTCTGCATGGAT
ACTTCGTCTTCGGGCCCACAGGATGCAATTTGGAGGGCTTCTTTGCCACCCTGG
GCGGTGAAATTGCCCTGTGGTCCTTGGTGGTCCTGGCCATCGAGCGGTACGTG
GTGGTGTGTAAGCCCATGAGCAACTTCCGCTTCGGGGAGAACCATGCCATCAT
GGGCGTTGCCTTCACCTGGGTCATGGCGCTGGCCTGCGCCGCACCCCCACTCG
CCGGCTGGTCCAGGTACATCCCCGAGGGCCTGCAGTGCTCGTGTGGAATCGAC
TACTACACGCTCAAGCCGGAGGTCAACAATGAATCCTTCGTGATTTATATGTTC
GTGGTCCACTTCACCATCCCCATGATTATCATCTTTTTCTGCTATGGGCAGCTC
GTCTTCACCGTCAAGGAGGCCGCTGCCCAGCAGCAGGAGTCAGCCACCACACA
GAAGGCAGAGAAGGAGGTCACCCGCATGGTCATCATCATGGTCATCGCTTTCC
TGATCTGCTGGGTGCCCTACGCCAGCGTGGCATTCTACATCTTCACCCACCAGG
GCTCCAACTTCGGTCCCATCTTCATGACCATCCCAGCGTTCTTTGCCAAGAGCG
CCGCCATCTACAACCCTGTCATCTATATCATGATGAACAAGCAGTTCCGGAACT
GCATGCTCACCACCATCTGCTGCGGCAAGAACCCACTGGGTGACGATGAGGCC
TCTGCTACCGTGTCCAAGACGGAGACGAGCCAGGTGGCCCCGGCCTAAAAGCT
TATCGATATCGATACCGTCGACTCGCTGATCAGCCTCGACTGTGCCTTCTAGTT
GCCAGCCATCTGTTGTTTGCCCCTCCCCCGTGCCTTCCTTGACCCTGGAAGGTG
CCACTCCCACTGTCCTTTCCTAATAAAATGAGGAAATTGCATCGCATTGTCTGA
GTAGGTGTCATTCTATTCTGGGGGGTGGGGTGGGGCAGGACAGCAAGGGGGA
GGATTGGGAAGACAATAGCAGGCATGCTGGGGATGCGGTGGGCTCTATGGCTT
CTGAGGCGGAAAGAACCAGCTGGGGCTCGACTAGAGCATGGCTACGTAGATA
AGTAGCATGGCGGGTTAATCATTAACTACAAGGAACCCCTAGTGATGGAGTTG
GCCACTCCCTCTCTGCGCGCTCGCTCGCTCACTGAGGCCGGGCGACCAAAGGT
CGCCCGACGCCCGGGCGGCCTCAGTGAGCGAGCGAGCGCGCAGAGCTTTTTGC
AAAAGCCTAGGCCTCCAAAAAAGCCTCCTCACTACTTCTGGAATAGCTCAGAG
GCCGAGGCGGCCTCGGCCTCTGCATAAATAAAAAAAATTAGTCAGCCATGGGG
CGGAGAATGGGCGGAACTGGGCGGAGTTAGGGGGGGGATGGGCGGAGTTAGG
GGCGGGACTATGGTTGCTGACTAATTGAGATGCATGCTTTGCATACTTCTGCCT
GCTGGGGAGCCTGGGGACTTTCCACACCTGGTTGCTGACTAATTGAGATGCAT
GCTTTGCATACTTCTGCCTGCTGGGGAGCCTGGGGACTTTCCACACCCTAACTG
ACACACATTCCACAGCTGCATTAATGAATCGGCCAACGCGCGGGGAGAGGCG
GTTTGCGTATTGGGCGCTCTTCCGCTTCCTCGCTCACTGACTCGCTGCGCTCGG
TCGTTCGGCTGCGGCGAGCGGTATCAGCTCACTCAAAGGCGGTAATACGGTTA
TCCACAGAATCAGGGGATAACGCAGGAAAGAACATGTGAGCAAAAGGCCAGC
AAAAGGCCAGGAACCGTAAAAAGGCCGCGTTGCTGGCGTTTTTCCATAGGCTC
CGCCCCCCTGACGAGCATCACAAAAATCGACGCTCAAGTCAGAGGTGGCGAA
ACCCGACAGGACTATAAAGATACCAGGCGTTTCCCCCTGGAAGCTCCCTCGTG
CGCTCTCCTGTTCCGACCCTGCCGCTTACCGGATACCTGTCCGCCTTTCTCCCTT
CGGGAAGCGTGGCGCTTTCTCATAGCTCACGCTGTAGGTATCTCAGTTCGGTGT
AGGTCGTTCGCTCCAAGCTGGGCTGTGTGCACGAACCCCCCGTTCAGCCCGAC
CGCTGCGCCTTATCCGGTAACTATCGTCTTGAGTCCAACCCGGTAAGACACGA
CTTATCGCCACTGGCAGCAGCCACTGGTAACAGGATTAGCAGAGCGAGGTATG
TAGGCGGTGCTACAGAGTTCTTGAAGTGGTGGCCTAACTACGGCTACACTAGA
AGAACAGTATTTGGTATCTGCGCTCTGCTGAAGCCAGTTACCTTCGGAAAAAG
AGTTGGTAGCTCTTGATCCGGCAAACAAACCACCGCTGGTAGCGGTGGTTTTTT
TGTTTGCAAGCAGCAGATTACGCGCAGAAAAAAAGGATCTCAAGAAGATCCTT
TGATCTTTTCTACGGGGTCTGACGCTCAGTGGAACGAAAACTCACGTTAAGGG
ATTTTGGTCATGAGATTATCAAAAAGGATCTTCACCTAGATCCTTTTAAATTAA
AAATGAAGTTTTAAATCAATCTAAAGTATATATGAGTAAACTTGGTCTGACAG
TTACCAATGCTTAATCAGTGAGGCACCTATCTCAGCGATCTGTCTATTTCGTTC
ATCCATAGTTGCCTGACTCCCCGTCGTGTAGATAACTACGATACGGGAGGGCT
TACCATCTGGCCCCAGTGCTGCAATGATACCGCGAGACCCACGCTCACCGGCT
CCAGATTTATCAGCAATAAACCAGCCAGCCGGAAGGGCCGAGCGCAGAAGTG
GTCCTGCAACTTTATCCGCCTCCATCCAGTCTATTAATTGTTGCCGGGAAGCTA
GAGTAAGTAGTTCGCCAGTTAATAGTTTGCGCAACGTTGTTGCCATTGCTACAG
GCATCGTGGTGTCACGCTCGTCGTTTGGTATGGCTTCATTCAGCTCCGGTTCCC
AACGATCAAGGCGAGTTACATGATCCCCCATGTTGTGCAAAAAAGCGGTTAGC
TCCTTCGGTCCTCCGATCGTTGTCAGAAGTAAGTTGGCCGCAGTGTTATCACTC
ATGGTTATGGCAGCACTGCATAATTCTCTTACTGTCATGCCATCCGTAAGATGC
TTTTCTGTGACTGGTGAGTACTCAACCAAGTCATTCTGAGAATAGTGTATGCGG
CGACCGAGTTGCTCTTGCCCGGCGTCAATACGGGATAATACCGCGCCACATAG
CAGAACTTTAAAAGTGCTCATCATTGGAAAACGTTCTTCGGGGCGAAAACTCT
CAAGGATCTTACCGCTGTTGAGATCCAGTTCGATGTAACCCACTCGTGCACCCA
ACTGATCTTCAGCATCTTTTACTTTCACCAGCGTTTCTGGGTGAGCAAAAACAG
GAAGGCAAAATGCCGCAAAAAAGGGAATAAGGGCGACACGGAAATGTTGAAT
ACTCATACTCTTCCTTTTTCAATATTATTGAAGCATTTATCAGGGTTATTGTCTC
ATGAGCGGATACATATTTGAATGTATTTAGAAAAATAAACAAATAGGGGTTCC
GCGCACATTTCCCCGAAAAGTGCCACCTGACGTCTAAGAAACCATTATTATCA
TGACATTAACCTATAAAAATAGGCGTATCACGAGGCCCTTTCGTCTCGCGCGTT
TCGGTGATGACGGTGAAAACCTCTGACACATGCAGCTCCCGGAGACGGTCACA
GCTTGTCTGTAAGCGGATGCCGGGAGCAGACAAGCCCGTCAGGGCGCGTCAGC
GGGTGTTGGCGGGTGTCGGGGCTGGCTTAACTATGCGGCATCAGAGCAGATTG
TACTGAGAGTGCACCATTCGACGCTCTCCCTTATGCGACTCCTGCATTAGGAAG
CAGCCCAGTAGTAGGTTGAGGCCGTTGAGCACCGCCGCCGCAAGGAATGGTGC
ATGCAAGGAGATGGCGCCCAACAGTCCCCCGGCCACGGGGCCTGCCACCATAC
CCACGCCGAAACAAGCGCTCATGAGCCCGAAGTGGCGAGCCCGATCTTCCCCA
TCGGTGATGTCGGCGATATAGGCGCCAGCAACCGCACCTGTGGCGCCGGTGAT
GCCGGCCACGATGCGTCCGGCGTAGAGGATCTGGCTAGCGATGACCCTGCTGA
TTGGTTCGCTGACCATTTCCGGGTGCGGGACGGCGTTACCAGAAACTCAGAAG
GTTCGTCCAACCAAACCGACTCTGACGGCAGTTTACGAGAGAGATGATAGGGT
CTGCTTCAGTAAGCCAGATGCTACACAATTAGGCTTGTACATATTGTCGTTAGA
ACGCGGCTACAATTAATACATAACCTTATGTATCATA

### Example 29—Additional Aspects of the Present Disclosure

Described herein are polynucleotides encoding a rhodopsin transgene. A polynucleotide encoding a rhodopsin transgene comprises a 5′ UTR and a coding region (i.e., CDS), wherein the coding region comprises a polynucleotide encoding a functional rhodopsin polypeptide.

In examples, the 5′UTR of the rhodopsin transgene consists of:
a 5′ end consisting of the sequence of nucleotides 1 to 423 of SEQ ID NO: 39;
a 3′ end consisting of the sequence of nucleotides 424 to 439 of SEQ ID NO: 39; and
a sequence inserted between nucleotides 1 to 423 and 424 to 439 of SEQ ID NO: 39, wherein the inserted sequence consists of SEQ ID NO: 26.

In examples, the sequence inserted between nucleotides 1 to 423 and 424 to 439 of SEQ ID NO: 39 consist of, in a 5′ to 3′ direction: (a) SEQ ID NO: 26, (b) ATC, (c) SEQ ID NO: 37, (d) SEQ ID NO 12, and (e) SEQ ID NO: 38.

In examples, the coding region of said rhodopsin transgene does not comprise the sequences of SEQ ID NOs: 5 or 7. In examples, the rhodopsin transgene coding region comprises the sequences of SEQ ID NO: 24 and SEQ ID NO: 25. In examples, the rhodopsin transgene coding region comprises the sequence of nucleotides 1819 to 2866 of SEQ ID NO: 28.

In examples, the 5′UTR of said rhodopsin transgene consists of the sequence of SEQ ID NO: 27 or SEQ ID NO: 72.

Also described herein are gene therapy vectors comprising polynucleotides described herein. In examples, a gene therapy vector comprises the sequence of SEQ ID NO: 28 or a variant of SEQ ID NO: 28 in which SEQ ID NO: 29 is deleted. In examples, a gene therapy vector comprises the nucleotide sequence of SEQ ID NO: 31. In examples, a vector comprises the nucleotide sequence of SEQ ID NO: 64.

Described herein are AAV viral particles comprising gene therapy vectors and polynucleotides described herein.

Described herein are pharmaceutical compositions comprising polynucleotides described herein and pharmaceutical compositions comprising gene therapy vectors described herein.

Described herein are methods of treating retinitis pigmentosa in a subject in need thereof, comprising administering a polynucleotide or gene therapy vector described herein.

1. A gene therapy vector that expresses a rhodopsin transgene;
wherein said transgene encodes functional rhodopsin;
wherein the coding region of said rhodopsin transgene does not comprise the sequences of SEQ ID NOs: 5 or 7;
and wherein the 5′UTR of the rhodopsin transgene consists of a 5′ end consisting of the sequence of nucleotides 1 to 423 of SEQ ID NO: 39;
a 3′ end consisting of the sequence of nucleotides 424 to 439 of SEQ ID NO: 39;
and a sequence inserted between nucleotides 1 to 423 and 424 to 439 of SEQ ID NO: 39;
wherein said inserted sequence consists of SEQ ID NO: 26.

2. The gene therapy vector of claim 1, wherein said rhodopsin transgene coding region comprises the sequences of SEQ ID NO: 24 and SEQ ID NO: 25.

3. The gene therapy vector of claim 2, wherein said rhodopsin transgene coding region comprises the sequence of nucleotides 1819 to 2866 of SEQ ID NO: 28.

4. The gene therapy vector of claim 1, wherein said 5′UTR of said rhodopsin transgene consists of the sequence of SEQ ID NO: 27.

5. The gene therapy vector of claim 1, wherein said gene therapy vector comprises the sequence of SEQ ID NO: 28 or a variant of SEQ ID NO: 28 in which SEQ ID NO: 29 is deleted.

6. The gene therapy vector of claim 1, wherein said gene therapy vector comprises the nucleotide sequence of SEQ ID NO: 31.

7. The gene therapy vector of claim 1, wherein said gene therapy vector comprises the nucleotide sequence of SEQ ID NO: 64.

8. A pharmaceutical composition comprising the gene therapy vector of claim 1.

9. A method for treating retinitis pigmentosa in a subject in need thereof, the method comprising administering to the subject the gene therapy vector of claim 1.

10. A gene therapy vector that expresses a rhodopsin transgene;
wherein said transgene encodes functional rhodopsin;
wherein the coding region of said rhodopsin transgene does not comprise the sequences of SEQ ID NOs: 5 or 7;
and wherein the 5′UTR of the rhodopsin transgene consists of a 5′ end consisting of the sequence of nucleotides 1 to 423 of SEQ ID NO: 39;
a 3′ end consisting of the sequence of nucleotides 424 to 439 of SEQ ID NO: 39;
and a sequence inserted between nucleotides 1 to 423 and 424 to 439 of SEQ ID NO: 39;
wherein said inserted sequence consists of, in a 5′ to 3′ direction: (a) SEQ ID NO: 26, (b) ATC, (c) SEQ ID NO: 37, (d) SEQ ID NO 12, and (e) SEQ ID NO: 38.

11. The gene therapy vector of claim 10, wherein the 5′UTR of the rhodopsin transgene consists of SEQ ID NO: 72.

12. The gene therapy vector of claim 10, wherein said rhodopsin transgene coding region comprises the sequences of SEQ ID NO: 24 and SEQ ID NO: 25.

13. The gene therapy vector of claim 12, wherein said rhodopsin transgene coding region comprises the sequence of nucleotides 1819 to 2866 of SEQ ID NO: 28.

14. A pharmaceutical composition comprising the gene therapy vector of claim 10.

15. A method for treating retinitis pigmentosa in a subject in need thereof, the method comprising administering to the subject the gene therapy vector of claim 10.
